SEQUENCE LISTING (151 sequences)

SEQ ID NO: 1 - DNA, 1818 bp, Synthetic construct
  source 1..1818: mol_type = other DNA
atggacacgtacgcgggtgcttacgaccgtcagtcgcgcgagcgcgaaaattcgagcgcagcaagcccagcgacacagcgtagcgccaacgaagacaaggcggccgaccttcagcgcgaagtcgagcgcgacgggggccggttcaggttcgtcgggcatttcagcgaagcgccgggcacgtcggcgttcgggacggcggagcgcccggagttcgaacgcatcctgaacgaatgccgcgccgggcggctcaacatgatcattgtctatgacgtgtcgcgcttctcgcgcctgaaggtcatggacgcgattccgattgtctcggaattgctcgccctgggcgtgacgattgtttccactcaggaaggcgtcttccggcagggaaacgtcatggacctgattcacctgattatgcggctcgacgcgtcgcacaaagaatcttcgctgaagtcggcgaagattctcgacacgaagaaccttcagcgcgaattgggcgggtacgtcggcgggaaggcgccttacggcttcgagcttgtttcggagacgaaggagatcacgcgcaacggccgaatggtcaatgtcgtcatcaacaagcttgcgcactcgaccactccccttaccggacccttcgagttcgagcccgacgtaatccggtggtggtggcgtgagatcaagacgcacaaacaccttcccttcaagccgggcagtcaagccgccattcacccgggcagcatcacggggctttgtaagcgcatggacgctgacgccgtgccgacccggggcgagacgattgggaagaagaccgcttcaagcgcctgggacccggcaaccgttatgcgaatccttcgggacccgcgtattgcgggcttcgccgctgaggtgatctacaagaagaagccggacggcacgccgaccacgaagattgagggttaccgcattcagcgcgacccgatcacgctccggccggtcgagcttgattgcggaccgatcatcgagcccgctgagtggtatgagcttcaggcgtggttggacggcagggggcgcggcaaggggctttcccgggggcaagccattctgtccgccatggacaagctgtactgcgagtgtggcgccgtcatgacttcgaagcgcggggaagaatcgatcaaggactcttaccgctgccgtcgccggaaggtggtcgacccgtccgcacctgggcagcacgaaggcacgtgcaacgtcagcatggcggcactcgacaagttcgttgcggaacgcatcttcaacaagatcaggcacgccgaaggcgacgaagagacgttggcgcttctgtgggaagccgcccgacgcttcggcaagctcactgaggcgcctgagaagagcggcgaacgggcgaaccttgttgcggagcgcgccgacgccctgaacgcccttgaagagctgtacgaagaccgcgcggcaggcgcgtacgacggacccgttggcaggaagcacttccggaagcaacaggcagcgctgacgctccggcagcaaggggcggaagagcggcttgccgaacttgaagccgccgaagccccgaagcttccccttgaccaatggttccccgaagacgccgacgctgacccgaccggccctaagtcgtggtgggggcgcgcgtcagtagacgacaagcgcgtgttcgtcgggctcttcgtagacaagatcgttgtcacgaagtcgactacgggcagggggcagggaacgcccatcgagaagcgcgcttcgatcacgtgggcgaagccgccgaccgacgacgacgaagacgacgcccaggacggcacggaagacgtagcggcgtag

SEQ ID NO: 2 - DNA, 2297 bp, Synthetic construct
  source 1..2297: mol_type = other DNA
atgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtcccgcggtatcattgcagcactggggccagatggtaagccctcccgtatcgtagttatctacacgacggggagtcaggcaactatggatgaacgaaatagacagatcgctgagataggtgcctcactgattaagcattggtaaggaggacaatcatgaacatgaacatcaaaaaaattgtaaaacaagccacagttctgacttttacgactgcacttctggcaggaggagcgactcaagccttcgcgaaagaaaataaccaaaaagcatacaaagaaacgtacggcgtttctcatattacacgccatgatatgctgcagatccctaaacagcagcaaaacgaaaaataccaagtgcctcaattcgatcaatcaacgattaaaaatattgagtctgcaaaaggacttgatgtgtgggacagctggccgctgcaaaacgctgacggaacagtagctgaatacaacggctatcacgttgtgtttgctcttgcgggaagcccgaaagacgctgatgacacatcaatctacatgttttatcaaaaggtcggcgacaactcaatcgacagctggaaaaacgcgggccgtgtctttaaagacagcgataagttcgacgccaacgatccgatcctgaaagatcagacgcaagaatggtccggttctgcaacctttacatctgacggaaaaatccgtttattctacactgactattccggtaaacattacggcaaacaaagcctgacaacagcgcaggtaaatgtgtcaaaatctgatgacacactcaaaatcaacggagtggaagatcacaaaacgatttttgacggagatggaaaaacatatcagaacgttcagcagtttatcgatgaaggcaattatacatccggcgacaaccatacgctgagagatcctcactacgttgaagacaaaggccataaataccttgtattcgaagccaacacgggaacagaaaacggataccaaggcgaagaatctttatttaacaaagcgtactacggcggcggcacgaacttcttccgtaaagaaagccagaagcttcagcagagcgctaaaaaacgcgatgctgagttagcgaacggcgccctcggtatcatagagttaaataatgattacacattgaaaaaagtaatgaagccgctgatcacttcaaacacggtaactgatgaaatcgagcgcgcgaatgttttcaaaatgaacggcaaatggtacttgttcactgattcacgcggttcaaaaatgacgatcgatggtattaactcaaacgatatttacatgcttggttatgtatcacactctttaaccggcccttacaagccgctgaacaaaacagggcttgtgctgcaaatgggtcttgatccaaacgatgtgacattcacttactctcacttcgcagtgccgcaagccaaaggcaacaatgtggttatcacaagctacatgacaaacagaggcttcttcgaggataaaaaggcaacatttgcgccaagcttcttaatgaacatcaaaggcaataaaacatccgttgtcaaaaacagcatcctggagcaaggacagctgacagtcaactaa

SEQ ID NO: 3 - DNA, 1439 bp, Synthetic construct
  source 1..1439: mol_type = other DNA
taaggaggacaatcatgaacatgaacatcaaaaaaattgtaaaacaagccacagttctgacttttacgactgcacttctggcaggaggagcgactcaagccttcgcgaaagaaaataaccaaaaagcatacaaagaaacgtacggcgtttctcatattacacgccatgatatgctgcagatccctaaacagcagcaaaacgaaaaataccaagtgcctcaattcgatcaatcaacgattaaaaatattgagtctgcaaaaggacttgatgtgtgggacagctggccgctgcaaaacgctgacggaacagtagctgaatacaacggctatcacgttgtgtttgctcttgcgggaagcccgaaagacgctgatgacacatcaatctacatgttttatcaaaaggtcggcgacaactcaatcgacagctggaaaaacgcgggccgtgtctttaaagacagcgataagttcgacgccaacgatccgatcctgaaagatcagacgcaagaatggtccggttctgcaacctttacatctgacggaaaaatccgtttattctacactgactattccggtaaacattacggcaaacaaagcctgacaacagcgcaggtaaatgtgtcaaaatctgatgacacactcaaaatcaacggagtggaagatcacaaaacgatttttgacggagatggaaaaacatatcagaacgttcagcagtttatcgatgaaggcaattatacatccggcgacaaccatacgctgagagatcctcactacgttgaagacaaaggccataaataccttgtattcgaagccaacacgggaacagaaaacggataccaaggcgaagaatctttatttaacaaagcgtactacggcggcggcacgaacttcttccgtaaagaaagccagaagcttcagcagagcgctaaaaaacgcgatgctgagttagcgaacggcgccctcggtatcatagagttaaataatgattacacattgaaaaaagtaatgaagccgctgatcacttcaaacacggtaactgatgaaatcgagcgcgcgaatgttttcaaaatgaacggcaaatggtacttgttcactgattcacgcggttcaaaaatgacgatcgatggtattaactcaaacgatatttacatgcttggttatgtatcacactctttaaccggcccttacaagccgctgaacaaaacagggcttgtgctgcaaatgggtcttgatccaaacgatgtgacattcacttactctcacttcgcagtgccgcaagccaaaggcaacaatgtggttatcacaagctacatgacaaacagaggcttcttcgaggataaaaaggcaacatttgcgccaagcttcttaatgaacatcaaaggcaataaaacatccgttgtcaaaaacagcatcctggagcaaggacagctgacagtcaactaa

SEQ ID NO: 4 - DNA, 50 bp, Synthetic construct
  source 1..50: mol_type = other DNA
agtagtgccccaactggggtaacctttgagttctctcagttgggggcgta

SEQ ID NO: 5 - DNA, 51 bp, Synthetic construct
  source 1..51: mol_type = other DNA
ccgcggtgcgggtgccagggcgtgcccttgggctccccgggcgcgtactcc

SEQ ID NO: 6 - DNA, 50 bp, Synthetic construct
  source 1..50: mol_type = other DNA
agtagtgccccaactggggtaacctccgagttctctcagttgggggcgta

SEQ ID NO: 7 - DNA, 51 bp, Synthetic construct
  source 1..51: mol_type = other DNA
ccgcggtgcgggtgccagggcgtgcccccgggctccccgggcgcgtactcc

SEQ ID NO: 8 - DNA, 34 bp, Synthetic construct
  source 1..34: mol_type = other DNA
gaagttcctatactttctagagaataggaacttc

SEQ ID NO: 9 - DNA, 21 bp, Synthetic construct
  source 1..21: mol_type = other DNA
ttaataacacattgcggacgt

SEQ ID NO: 10 - DNA, 34 bp, Synthetic construct
  source 1..34: mol_type = other DNA
gggcactactagaattcagtacattaaaaacgtc

SEQ ID NO: 11 - DNA, 45 bp, Synthetic construct
  source 1..45: mol_type = other DNA
actgaattctagtagtgccccaactggggtaacctttgagttctc

SEQ ID NO: 12 - DNA, 57 bp, Synthetic construct
  source 1..57: mol_type = other DNA
ctctagaaagtataggaacttctacgcccccaactgagagaactcaaaggttacccc

SEQ ID NO: 13 - DNA, 54 bp, Synthetic construct
  source 1..54: mol_type = other DNA
gaagttcctatactttctagagaataggaacttcgaattcgtagggataacagg

SEQ ID NO: 14 - DNA, 21 bp, Synthetic construct
  source 1..21: mol_type = other DNA
cggagaattaagggaattacc

SEQ ID NO: 15 - DNA, 54 bp, Synthetic construct
  source 1..54: mol_type = other DNA
taaacataacgaattcagtagtgccccaactggggtaacctccgagttctctca

SEQ ID NO: 16 - DNA, 54 bp, Synthetic construct
  source 1..54: mol_type = other DNA
tgtgggtctctctcctgagacgtacgcccccaactgagagaactcggaggttac

SEQ ID NO: 17 - DNA, 42 bp, Synthetic construct
  source 1..42: mol_type = other DNA
ggtctctcgctgtcatgagacgagtagtgccccaactggggt

SEQ ID NO: 18 - DNA, 38 bp, Synthetic construct
  source 1..38: mol_type = other DNA
tatcctgtcagaattcgaagttcctattctctagaaag

SEQ ID NO: 19 - DNA, 36 bp, Synthetic construct
  source 1..36: mol_type = other DNA
taaacataacaagcttccgcggtgcgggtgccaggg

SEQ ID NO: 20 - DNA, 42 bp, Synthetic construct
  source 1..42: mol_type = other DNA
ggtctctctcctgactgagacgggagtacgcgcccggggagc

SEQ ID NO: 21 - DNA, 42 bp, Synthetic construct
  source 1..42: mol_type = other DNA
ctggggtctctcgcttgagacgccgcggtgcgggtgccaggg

SEQ ID NO: 22 - DNA, 38 bp, Synthetic construct
  source 1..38: mol_type = other DNA
tatcctgtcaaagcttgaagttcctattctctagaaag

SEQ ID NO: 23 - DNA, 52 bp, Synthetic construct
  source 1..52: mol_type = other DNA
taaacataacggatccccgcggtgcgggtgccagggcgtgcccttgggctcc

SEQ ID NO: 24 - DNA, 57 bp, Synthetic construct
  source 1..57: mol_type = other DNA
tgtgcgtctcactcctgagaccggagtacgcgcccggggagcccaagggcacgccct

SEQ ID NO: 25 - DNA, 58 bp, Synthetic construct
  source 1..58: mol_type = other DNA
cgtctcacgctgtcatgagaccccgcggtgcgggtgccagggcgtgcccccgggctcc

SEQ ID NO: 26 - DNA, 46 bp, Synthetic construct
  source 1..46: mol_type = other DNA
taggaacttcggagtacgcgcccggggagcccgggggcacgccctg

SEQ ID NO: 27 - DNA, 32 bp, Synthetic construct
  source 1..32: mol_type = other DNA
cgcgtactccgaagttcctatactttctagag

SEQ ID NO: 28 - DNA, 38 bp, Synthetic construct
  source 1..38: mol_type = other DNA
tatcctgtcaggatccgaagttcctattctctagaaag

SEQ ID NO: 29 - DNA, 58 bp, Synthetic construct; identical to SEQ ID NO: 25
  source 1..58: mol_type = other DNA

SEQ ID NO: 30 - DNA, 38 bp, Synthetic construct; identical to SEQ ID NO: 28
  source 1..38: mol_type = other DNA

SEQ ID NO: 31 - DNA, 38 bp, Synthetic construct
  source 1..38: mol_type = other DNA
tgtaaacataacgatatcagtagtgccccaactggggt

SEQ ID NO: 32 - DNA, 35 bp, Synthetic construct
  source 1..35: mol_type = other DNA
ctgactgagaccgattacgcccccaactgagagaa

SEQ ID NO: 33 - DNA, 35 bp, Synthetic construct
  source 1..35: mol_type = other DNA
acgcttgagaccgatagtagtgccccaactggggt

SEQ ID NO: 34 - DNA, 40 bp, Synthetic construct
  source 1..40: mol_type = other DNA
tatatcctgtcagatatcgaagttcctattctctagaaag

SEQ ID NO: 35 - DNA, 34 bp, Synthetic construct
  source 1..34: mol_type = other DNA
atattgtggtgtaaacataacgaattcagtagtg

SEQ ID NO: 36 - DNA, 31 bp, Synthetic construct
  source 1..31: mol_type = other DNA
acccgccaatatatcctgtcagaattcgaag

SEQ ID NO: 37 - DNA, 31 bp, Synthetic construct
  source 1..31: mol_type = other DNA
atattgtggtgtaaacataacaagcttccgc

SEQ ID NO: 38 - DNA, 32 bp, Synthetic construct
  source 1..32: mol_type = other DNA
acccgccaatatatcctgtcaaagcttgaagt

SEQ ID NO: 39 - DNA, 20 bp, Synthetic construct
  source 1..20: mol_type = other DNA
cgtctcaggagagagaccca

SEQ ID NO: 40 - DNA, 20 bp, Synthetic construct
  source 1..20: mol_type = other DNA
cgtctcatgacagcgagaga

SEQ ID NO: 41 - DNA, 20 bp, Synthetic construct
  source 1..20: mol_type = other DNA
cgtctcagtcaggagagaga

SEQ ID NO: 42 - DNA, 21 bp, Synthetic construct
  source 1..21: mol_type = other DNA
cgtctcaagcgagagacccca

SEQ ID NO: 43 - DNA, 39 bp, Synthetic construct
  source 1..39: mol_type = other DNA
cgtacgtctcaggagtgccagagacccacagcttgtctg

SEQ ID NO: 44 - DNA, 39 bp, Synthetic construct
  source 1..39: mol_type = other DNA
tactcgtctcatgactcccagagaccccagctggcacga

SEQ ID NO: 45 - DNA, 39 bp, Synthetic construct
  source 1..39: mol_type = other DNA
ctcccgtctcagtcatgccagagacccacagcttgtctg

SEQ ID NO: 46 - DNA, 39 bp, Synthetic construct
  source 1..39: mol_type = other DNA
gcggcgtctcaagcgtcccagagaccccagctggcacga

SEQ ID NO: 47 - DNA, 37 bp, Synthetic construct
  source 1..37: mol_type = other DNA
gcgccgtctcgctcgaatgagtattcaacatttccgt

SEQ ID NO: 48 - DNA, 31 bp, Synthetic construct
  source 1..31: mol_type = other DNA
gcgccgtctcggggacccacgctcaccggct

SEQ ID NO: 49 - DNA, 31 bp, Synthetic construct
  source 1..31: mol_type = other DNA
gcgccgtctcgtcccgcggtatcattgcagc

SEQ ID NO: 50 - DNA, 42 bp, Synthetic construct
  source 1..42: mol_type = other DNA
gcgccgtctcgctcaaagcttaccaatgcttaatcagtgagg

SEQ ID NO: 51 - DNA, 60 bp, Synthetic construct
  source 1..60: mol_type = other DNA
aaatactgtagaaaagaggaaggaaataataagttctgttatgagtattcaacatttccg

SEQ ID NO: 52 - DNA, 60 bp, Synthetic construct
  source 1..60: mol_type = other DNA
aaactcacgttaagggattttggtcatgcattctaggtgattaccaatgcttaatcagtg

SEQ ID NO: 53 - DNA, 35 bp, Synthetic construct
  source 1..35: mol_type = other DNA
gcgccgtctcgctcgtcgagcttgacattgtagga

SEQ ID NO: 54 - DNA, 32 bp, Synthetic construct
  source 1..32: mol_type = other DNA
gcgccgtctcgaaacgccgtacgtttctttgt

SEQ ID NO: 55 - DNA, 33 bp, Synthetic construct
  source 1..33: mol_type = other DNA
gcgccgtctcggtttctcatattacacgccatg

SEQ ID NO: 56 - DNA, 34 bp, Synthetic construct
  source 1..34: mol_type = other DNA
gcgccgtctcgcatctccgtcaaaaatcgttttg

SEQ ID NO: 57 - DNA, 35 bp, Synthetic construct
  source 1..35: mol_type = other DNA
gcgccgtctcggatggaaaaacatatcagaacgtt

SEQ ID NO: 58 - DNA, 31 bp, Synthetic construct
  source 1..31: mol_type = other DNA
gcgccgtctcgggatctctcagcgtatggtt

SEQ ID NO: 59 - DNA, 32 bp, Synthetic construct
  source 1..32: mol_type = other DNA
gcgccgtctcgatcctcactacgttgaagaca

SEQ ID NO: 60 - DNA, 39 bp, Synthetic construct
  source 1..39: mol_type = other DNA
gcgccgtctcgctcaaagcggtataggaacttcccttag

SEQ ID NO: 61 - DNA, 60 bp, Synthetic construct
  source 1..60: mol_type = other DNA
aaatactgtagaaaagaggaaggaaataataagttctgttatgagccatattcaacggga

SEQ ID NO: 62 - DNA, 40 bp, Synthetic construct
  source 1..40: mol_type = other DNA
gttcatgattgtcctccttagaaaaactcatcgagcatca

SEQ ID NO: 63 - DNA, 45 bp, Synthetic construct
  source 1..45: mol_type = other DNA
taaggaggacaatcatgaacatgaacatcaaaaaaattgtaaaac

SEQ ID NO: 64 - DNA, 60 bp, Synthetic construct
  source 1..60: mol_type = other DNA
aaactcacgttaagggattttggtcatgcattctaggtgattagttgactgtcagctgtc

SEQ ID NO: 65 - DNA, 60 bp, Synthetic construct; identical to SEQ ID NO: 51
  source 1..60: mol_type = other DNA

SEQ ID NO: 66 - DNA, 60 bp, Synthetic construct
  source 1..60: mol_type = other DNA
acaatttttttgatgttcatgttcatgattgtcctccttaccaatgcttaatcagtgagg

SEQ ID NO: 67 - DNA, 59 bp, Synthetic construct
  source 1..59: mol_type = other DNA
aaatactgtagaaaagaggaaggaaataataagttctgttccagccaggacagaaatgc

SEQ ID NO: 68 - DNA, 60 bp, Synthetic construct
  source 1..60: mol_type = other DNA
acaatttttttgatgttcatgttcatgattgtcctccttatttgccgactaccttggtga

SEQ ID NO: 69 - DNA, 25 bp, Synthetic construct
  source 1..25: mol_type = other DNA
aacagaacttattatttccttcctc

SEQ ID NO: 70 - DNA, 21 bp, Synthetic construct
  source 1..21: mol_type = other DNA
gatctattcagatagcagctc

SEQ ID NO: 71 - DNA, 21 bp, Synthetic construct
  source 1..21: mol_type = other DNA
gagctgctatctgaatagatc

SEQ ID NO: 72 - DNA, 20 bp, Synthetic construct
  source 1..20: mol_type = other DNA
tcacctagaatgcatgacca

SEQ ID NO: 73 - DNA, 59 bp, Synthetic construct
  source 1..59: mol_type = other DNA
aaatactgtagaaaagaggaaggaaataataagttctgttatgcgctcacgcaactggt

SEQ ID NO: 74 - DNA, 60 bp, Synthetic construct; identical to SEQ ID NO: 64
  source 1..60: mol_type = other DNA

SEQ ID NO: 75 - DNA, 69 bp, Synthetic construct
  source 1..69: mol_type = other DNA
gcgggcttatgagaaagaaattttgagtcgccgcgggtaaggaggacaatcggaggtggaggtggagct

SEQ ID NO: 76 - DNA, 60 bp, Synthetic construct
  source 1..60: mol_type = other DNA
cgtaggccggataaggcgctcgcgccgcatccggcagtgtggccccagcggccgcagcag

SEQ ID NO: 77 - DNA, 60 bp, Synthetic construct
  source 1..60: mol_type = other DNA
agaaagaaattttgagtcgccgcgggtaaggaggacaatcatggacacgtacgcgggtgc

SEQ ID NO: 78 - DNA, 60 bp, Synthetic construct
  source 1..60: mol_type = other DNA
cgtaggccggataaggcgctcgcgccgcatccggcagtgtctacgccgctacgtcttccg

SEQ ID NO: 79 - DNA, 79 bp, Synthetic construct
  source 1..79: mol_type = other DNA
tggtgattattgtcgccgctaacatcgtcggcatcggcatggcgaattaaggaggacaatcggaggtggaggtggagct

SEQ ID NO: 80 - DNA, 74 bp, Synthetic construct
  source 1..74: mol_type = other DNA
accgggatgacgcccagccagtggcgtcatctcaattcgcagaaagattaggccccagcggccgcagcagcacc

SEQ ID NO: 81 - DNA, 75 bp, Synthetic construct
  source 1..75: mol_type = other DNA
gtcgccgctaacatcgtcggcatcggcatggcgaattaaggaggacaatcatggacacgtacgcgggtgcttacg

SEQ ID NO: 82 - DNA, 71 bp, Synthetic construct
  source 1..71: mol_type = other DNA
accgggatgacgcccagccagtggcgtcatctcaattcgcagaaagattaactacgccgctacgtcttccg

SEQ ID NO: 83 - DNA, 39 bp, Synthetic construct
  source 1..39: mol_type = other DNA
gcgccgtctcgctcgggagtcgacttgccttccgcacaa

SEQ ID NO: 84 - DNA, 36 bp, Synthetic construct
  source 1..36: mol_type = other DNA
gcgccgtctcgctcacaatcactacttcgactctag

SEQ ID NO: 85 - DNA, 38 bp, Synthetic construct
  source 1..38: mol_type = other DNA
gcgccgtctcgctcgattgactggttggtgagagaaga

SEQ ID NO: 86 - DNA, 58 bp, Synthetic construct
  source 1..58: mol_type = other DNA
gactggttggtgagagaagagttttagagctagaaatagcaagttaaaataaggctag

SEQ ID NO: 87 - DNA, 42 bp, Synthetic construct
  source 1..42: mol_type = other DNA
gcgccgtctcgctcattgcaaaaaaaagggaagactccccag

SEQ ID NO: 88 - DNA, 38 bp, Synthetic construct
  source 1..38: mol_type = other DNA
gcgccgtctcgctcggcaaaattttccagatcgatttc

SEQ ID NO: 89 - DNA, 43 bp, Synthetic construct
  source 1..43: mol_type = other DNA
gcgccgtctcgctcaagcgttattggtttatctcatcggaact

SEQ ID NO: 90 - DNA, 40 bp, Synthetic construct
  source 1..40: mol_type = other DNA
gcgccgtctcgctcgggagtgtttgaatggatccagttcg

SEQ ID NO: 91 - DNA, 45 bp, Synthetic construct
  source 1..45: mol_type = other DNA
gcgccgtctcgctcaatggtttcagaaatgaaaggtaaaaaagag

SEQ ID NO: 92 - DNA, 40 bp, Synthetic construct
  source 1..40: mol_type = other DNA
gcgccgtctcgctcgccatatgtctaagggtgaagagttg

SEQ ID NO: 93 - DNA, 43 bp, Synthetic construct
  source 1..43: mol_type = other DNA
gcgccgtctcgctcaaagcttacttgtaaagttcattcatgcc

SEQ ID NO: 94 - DNA, 45 bp, Synthetic construct
  source 1..45: mol_type = other DNA
gcgccgtctcactcggcttaaatcaccagtctctctctacaaatc

SEQ ID NO: 95 - DNA, 43 bp, Synthetic construct
  source 1..43: mol_type = other DNA
gcgccgtctcactcaagcgctggattttggttttaggaattag

SEQ ID NO: 96 - DNA, 39 bp, Synthetic construct
  source 1..39: mol_type = other DNA
gcgccgtctcgctcgggagactagagccaagctgatctc

SEQ ID NO: 97 - DNA, 47 bp, Synthetic construct
  source 1..47: mol_type = other DNA
gcgccgtctcgctcaatggcagcgtgtcctctccaaatgaaatgaac

SEQ ID NO: 98 - DNA, 59 bp, Synthetic construct
  source 1..59: mol_type = other DNA
gcgccgtctcgctcgccatgcccaagaagaagaggaaggtgtctgatctcgtgctcgga

SEQ ID NO: 99 - DNA, 42 bp, Synthetic construct
  source 1..42: mol_type = other DNA
gcgccgtctcgctcacgaaccgaaatcgagcttaggcttatc

SEQ ID NO: 100 - DNA, 31 bp, Synthetic construct
  source 1..31: mol_type = other DNA
ctcggactcgctatcggaatcggatctgttg

SEQ ID NO: 101 - DNA, 30 bp, Synthetic construct
  source 1..30: mol_type = other DNA
attccgatagcgagtccgagcacgagatca

SEQ ID NO: 102 - DNA, 32 bp, Synthetic construct
  source 1..32: mol_type = other DNA
gaagtggatgctatcctcccactctcaatcac

SEQ ID NO: 103 - DNA, 32 bp, Synthetic construct
  source 1..32: mol_type = other DNA
tgggaggatagcatccacttcgaactggttag

SEQ ID NO: 104 - DNA, 37 bp, Synthetic construct
  source 1..37: mol_type = other DNA
gcgccgtctcgctcgttcggatcctaaaaagaagcga

SEQ ID NO: 105 - DNA, 36 bp, Synthetic construct
  source 1..36: mol_type = other DNA
gcgccgtctcgctcaaagctcagcgtttgcgttctt

SEQ ID NO: 106 - DNA, 58 bp, Synthetic construct
  source 1..58: mol_type = other DNA
gcgccgtctcgctcgattgaatcttgcagaagctacaaagataaggcttcatgccgaa

SEQ ID NO: 107 - DNA, 56 bp, Synthetic construct
  source 1..56: mol_type = other DNA
cgaaaacaccctgccataaaatgacagggtgttgatttcggcatgaagccttatct

SEQ ID NO: 108 - DNA, 38 bp, Synthetic construct
  source 1..38: mol_type = other DNA
gcgccgtctcgctcgattgcagaagctacaaagataag

SEQ ID NO: 109 - DNA, 20 bp, Synthetic construct
  source 1..20: mol_type = other DNA
caccctgccataaaatgaca

SEQ ID NO: 110 - DNA, 38 bp, Synthetic construct
  source 1..38: mol_type = other DNA
gtctttgtactctggtaccagaagctacaaagataagg

SEQ ID NO: 111 - DNA, 20 bp, Synthetic construct
  source 1..20: mol_type = other DNA
tgtcattttatggcagggtg

SEQ ID NO: 112 - DNA, 40 bp, Synthetic construct
  source 1..40: mol_type = other DNA
tgtcattttatggcagggtggggagcacatgaggaatcac

SEQ ID NO: 113 - DNA, 42 bp, Synthetic construct
  source 1..42: mol_type = other DNA
ctggggagtcttcccttttttttgcaatgagcgagacggcgc

SEQ ID NO: 114 - DNA, 58 bp, Synthetic construct
  source 1..58: mol_type = other DNA
gcgccgtctcgctcgattgaagtggaggttgctccaccgtctttgtactctggtacca

SEQ ID NO: 115 - DNA, 42 bp, Synthetic construct; identical to SEQ ID NO: 113
  source 1..42: mol_type = other DNA

SEQ ID NO: 116 - DNA, 47 bp, Synthetic construct
  source 1..47: mol_type = other DNA
tctgcggtggagcaacctccacttcacatttggttaatccaaccaag

SEQ ID NO: 117 - DNA, 43 bp, Synthetic construct
  source 1..43: mol_type = other DNA
tggaggttgctccaccgcagaaagctagtgtgaagtgtctggc

SEQ ID NO: 118 - DNA, 39 bp, Synthetic construct
  source 1..39: mol_type = other DNA
gcgccgtctcgctcgccatggtgagtaagggtgaagaag

SEQ ID NO: 119 - DNA, 40 bp, Synthetic construct
  source 1..40: mol_type = other DNA
gcgccgtctcgctcaaagctcaatagagctcatccatacc

SEQ ID NO: 120 - DNA, 36 bp, Synthetic construct
  source 1..36: mol_type = other DNA
gcgcggtctcgggagactaaatggagcaacctactg

SEQ ID NO: 121 - DNA, 37 bp, Synthetic construct
  source 1..37: mol_type = other DNA
gcgcggtctcgcatctcctttcttaatatatctaaca

SEQ ID NO: 122 - DNA, 32 bp, Synthetic construct
  source 1..32: mol_type = other DNA
gcgcggtctcggatgagaagtcacgtgtcaat

SEQ ID NO: 123 - DNA, 35 bp, Synthetic construct
  source 1..35: mol_type = other DNA
gcgcggtctcgagcggttctagaatgtcgcggaac

SEQ ID NO: 124 - DNA, 20 bp, Synthetic construct
  source 1..20: mol_type = other DNA
caaattgacgcttagacaac

SEQ ID NO: 125 - DNA, 47 bp, Synthetic construct
  source 1..47: mol_type = other DNA
gcgccgtctcgctcacgaaccataaataccagaattagcagcaatag

SEQ ID NO: 126 - DNA, 39 bp, Synthetic construct; identical to SEQ ID NO: 118
  source 1..39: mol_type = other DNA

SEQ ID NO: 127 - DNA, 21 bp, Synthetic construct
  source 1..21: mol_type = other DNA
cggagaattaagggaattacc

SEQ ID NO: 128 - DNA, 28 bp, Synthetic construct
  source 1..28: mol_type = other DNA
cgaaggcatgaatgaactttacaagtaa

SEQ ID NO: 129 - DNA, 30 bp, Synthetic construct; identical to SEQ ID NO: 101
  source 1..30: mol_type = other DNA

SEQ ID NO: 130 - DNA, 39 bp, Synthetic construct; identical to SEQ ID NO: 118
  source 1..39: mol_type = other DNA

SEQ ID NO: 131 - DNA, 19 bp, Synthetic construct
  source 1..19: mol_type = other DNA
cggataaaccttttcacgc

SEQ ID NO: 132 - DNA, 45 bp, Synthetic construct; identical to SEQ ID NO: 94
  source 1..45: mol_type = other DNA

SEQ ID NO: 133 - DNA, 35 bp, Synthetic construct; identical to SEQ ID NO: 123
  source 1..35: mol_type = other DNA

SEQ ID NO: 134 - DNA, 22 bp, Synthetic construct
  source 1..22: mol_type = other DNA
gtatcgagtggtgattttgtgc

SEQ ID NO: 135 - DNA, 47 bp, Synthetic construct; identical to SEQ ID NO: 125
  source 1..47: mol_type = other DNA

SEQ ID NO: 136 - DNA, 30 bp, Synthetic construct
  source 1..30: mol_type = other DNA
agcgatgagtagagacccacagcttgtctg

SEQ ID NO: 137 - DNA, 47 bp, Synthetic construct; identical to SEQ ID NO: 125
  source 1..47: mol_type = other DNA

SEQ ID NO: 138 - DNA, 32 bp, Synthetic construct; identical to SEQ ID NO: 122
  source 1..32: mol_type = other DNA

SEQ ID NO: 139 - DNA, 47 bp, Synthetic construct; identical to SEQ ID NO: 125
  source 1..47: mol_type = other DNA

SEQ ID NO: 140 - DNA, 20 bp, Synthetic construct
  source 1..20: mol_type = other DNA
gtcctgctggaatttgtgac

SEQ ID NO: 141 - DNA, 47 bp, Synthetic construct; identical to SEQ ID NO: 125
  source 1..47: mol_type = other DNA

SEQ ID NO: 142 - DNA, 39 bp, Synthetic construct; identical to SEQ ID NO: 118
  source 1..39: mol_type = other DNA

SEQ ID NO: 143 - DNA, 19 bp, Synthetic construct
  source 1..19: mol_type = other DNA
cggataaaccttttcacgc

SEQ ID NO: 144 - DNA, 23 bp, Synthetic construct
  source 1..23: mol_type = other DNA
gactggttggtgagagaagaagg

SEQ ID NO: 145 - DNA, 27 bp, Synthetic construct
  source 1..27: mol_type = other DNA
gaagtggaggttgctccaccgcagaaa

SEQ ID NO: 146 - DNA, 186 bp, Synthetic construct
  source 1..186: mol_type = other DNA
gaattcagtagtgccccaactggggtaacctccgagttctctcagttgggggcgtacgtctcaggagagagaccggtctctcgctgtcatgagacgagtagtgccccaactggggtaacctttgagttctctcagttgggggcgtagaagttcctatactttctagagaataggaacttcgaattc

SEQ ID NO: 147 - DNA, 188 bp, Synthetic construct
  source 1..188: mol_type = other DNA
ggatccccgcggtgcgggtgccagggcgtgcccttgggctccccgggcgcgtactccggtctcaggagtgagacgcgtctcacgctgtcatgagaccccgcggtgcgggtgccagggcgtgcccccgggctccccgggcgcgtactccgaagttcctatactttctagagaataggaacttcggatcc

SEQ ID NO: 148 - DNA, 187 bp, Synthetic construct
  source 1..187: mol_type = other DNA
aagcttccgcggtgcgggtgccagggcgtgcccttgggctccccgggcgcgtactcccgtctcagtcaggagagagaccggtctctcgcttgagacgccgcggtgcgggtgccagggcgtgccccgggctccccgggcgcgtactccgaagttcctatactttctagagaataggaacttcaagctt

SEQ ID NO: 149 - DNA, 192 bp, Synthetic construct
  source 1..192: mol_type = other DNA
gatatcagtagtgccccaactggggtaacctccgagttctctcagttgggggcgtaatcggtctcagtcaggagtgagacgcgtctcacgcttgagaccgatagtagtgccccaactggggtaacctttgagttctctcagttgggggcgtagaagttcctatactttctagagaataggaacttcgatatc

SEQ ID NO: 150 - DNA, 190 bp, Synthetic construct
  source 1..190: mol_type = other DNA
gaattcagtagtgccccaactggggtaacctccgagttctctcagttgggggcgtacgtctcaggagtgccagagaccggtctctgggagtcatgagacgagtagtgccccaactggggtaacctttgagttctctcagttgggggcgtagaagttcctatactttctagagaataggaacttcgaattc

SEQ ID NO: 151 - DNA, 192 bp, Synthetic construct
  source 1..192: mol_type = other DNA
aagcttccgcggtgcgggtgccagggcgtgcccttgggctccccgggcgcgtactcccgtctcagtcatgccagagaccggtctctgggacgcttgagacgccgcggtgcgggtgccagggcgtgcccccgggctccccgggcgcgtactccgaagttcctatactttctagagaataggaacttcaagctt

(19) United States
(12) Patent Application Publication
(10) Pub. No.: US 2025/0270571 A1
(43) Pub. Date: Aug. 28, 2025

(54) COMPOSITIONS AND METHODS RELATED TO GENE STACKING SYSTEMS

(71) Applicant: North Carolina State University, Raleigh, NC (US)

(72) Inventors: Anna Stepanova, Raleigh, NC (US); Jose Alonso Bellver, Raleigh, NC (US); Chengsong Zhao, Raleigh, NC (US)

(21) Appl. No.: 19/067,465

(22) Filed: Feb. 28, 2025

Related U.S. Application Data
(60) Provisional application No. 63/558,983, filed on Feb. 28, 2024.

(51) Int. Cl. C12N 15/82; C12N 1/20; C12R 1/19

(52) U.S. Cl. CPC C12N 15/8205; C12N 1/20; C12N 15/8213; C12R 2001/19

(57) ABSTRACT

The present disclosure provides materials and methods related to gene stacking. In particular, the present disclosure provides gene stacking systems and components thereof and methods for delivering target DNA inserts to acceptor vectors and target cells such as plant cells. The provided gene stacking systems and components thereof enable an increased DNA cargo size limit, such as an acceptor vector configured to contain up to 300 kb of target DNA inserts. The provided gene stacking systems and components thereof also enable editing of constructs post-assembly. Additionally, the provided gene stacking systems are compatible with all major Type IIS cloning technologies in plants.

### CROSS REFERENCE TO RELATED APPLICATIONS

This application claims priority to and the benefit of U.S. Provisional Patent Application No. 63/558,983 filed Feb. 28, 2024, which is incorporated herein by reference in its entirety for all purposes.

### GOVERNMENT SUPPORT

This invention was made with government support under grant number IOS1750006 awarded by the National Science Foundation. The government has certain rights in the invention.

### INCORPORATION-BY-REFERENCE OF MATERIAL SUBMITTED ELECTRONICALLY

Incorporation by reference in its entirety herein is a computer-readable nucleotide/amino acid sequence listing submitted concurrently herewith and identified as follows: 143,450 byte XML file named “NCSU_42876_202_SequenceListing” created on Feb. 28, 2025.

### FIELD

The present disclosure provides materials and methods related to gene stacking. In particular, the present disclosure provides gene stacking systems and components thereof and methods for delivering target DNA inserts to acceptor vectors and target cells such as plant cells.

### BACKGROUND

One of the technical hurdles that make the implementation of synthetic biology methods in plants cumbersome is the cargo size limit of existing vector systems. For example, the GoldenBraid (GB) technology many groups employ to build synthetic genetic circuits in E. coli (prior to transforming these constructs into plants using Agrobacterium-mediated transformation) has a limit of about 25 kilobases (kb). However, the designs of some multi-gene pathways or circuits exceed the 25 kb limit. Another major bottleneck in synthetic biology is the inability to modify large, multi-gene constructs after they have been built, often requiring researchers to start from entry-level DNA parts to remake the constructs even when only minor modifications are required (e.g., a point mutation needs to be introduced in the promoter or a subcellular localization tag needs to be swapped). Thus, there is a need for improved methods and tools for synthetic biology applications in plants.

### SUMMARY

Embodiments of the present disclosure include gene stacking systems and methods of gene stacking (e.g., DNA Assembly and Stacking Hybrid (DASH) system).

In some embodiments, the present disclosure provides a gene stacking system comprising: (a) at least one donor vector for delivering target DNA inserts; (b) a first acceptor vector configured to contain up to about 300 kb of target DNA inserts; and (c) a first engineered bacterial cell comprising the first acceptor vector. In some embodiments, the gene stacking system comprises: (i) two donor vectors; (ii) four donor vectors; or (iii) eight donor vectors. In some embodiments, the gene stacking system further comprises a second acceptor vector configured to contain up to about 300 kb of target DNA inserts and a second engineered bacterial cell comprising the second acceptor vector. In some embodiments, the second engineered bacterial cell comprises a recombineering system. In some embodiments, the first engineered bacterial cell comprises a recombineering system. In some embodiments, the recombincering system enables editing of the target DNA inserts and/or the acceptor vector. In some embodiments, the recombincering system comprises heat-shock-inducible lambda Red proteins. In some embodiments, the second engineered bacterial cell comprises an arabinose-inducible FLP recombinase. In some embodiments, the first engineered bacterial cell comprises an arabinose-inducible FLP recombinase. In some embodiments, the second engineered bacterial cell comprises an inducible PhiC31 recombinase or a rhamnose-inducible PhiC3/recombinase. In some embodiments, the first engineered bacterial cell comprises an inducible PhiC3/recombinase or a rhamnose-inducible PhiC31 recombinase. In some embodiments, the second engineered bacterial cell is derived from an E. coli SW105 strain. In some embodiments, the first engineered bacterial cell is derived from an E. coli SW105 strain. In some embodiments, the first engineered bacterial cell comprises a PhiC31 gene inserted immediately downstream of rhaA in the rhaBAD operon of the E. coli SW 105 genome. In some embodiments, the second engineered bacterial cell comprises a PhiC31 gene inserted immediately downstream of rhaT in the E. coli SW105 genome to form an operon with rhaT. In some embodiments, the second acceptor vector comprises an attP^{TT} site, an FRT site, and a selection marker. In some embodiments, the first acceptor vector comprises an attP^{TT} site, an FRT site, and a selection marker. In some embodiments, the selection marker is an antibiotic resistance gene. In some embodiments, the first acceptor vector comprises a kanamycin resistance gene. In some embodiments, the second acceptor vector comprises an ampicillin resistance gene. In some embodiments, each of the donor vectors comprises a first att site, a second att site, and an FRT site. In some embodiments, each of the donor vectors comprises a SacB gene. In some embodiments, each of the donor vectors comprises a selection marker. In some embodiments, the selection marker is an antibiotic resistance gene. In some embodiments, the antibiotic resistance gene is a kanamycin resistance gene, an ampicillin resistance gene, or a spectinomycin resistance gene. In some embodiments, the system is compatible with a GV3101 strain of Agrobacterium or a GV3101 derivate with a deletion in the mltB3 gene. In some embodiments, each of the donor vectors is either a Donor I vector or a Donor II vector and the system is configured so that delivery of the target DNA inserts comprises introducing a Donor I vector or a Donor II vector into the first engineered bacterial cell or the second engineered bacterial cell. In some embodiments, multiple target DNA inserts are introduced into the first or second engineered bacterial cell and each target DNA insert is introduced from a Donor I vector or a Donor II vector in an alternating pattern. In some embodiments, the gene stacking system is compatible with Type IIS restriction enzyme-based cloning technologies in plants. In some embodiments, each of the donor vectors is configured to incorporate target DNA inserts from: (i) Golden Gate vectors, GoldenBraid plasmids, Mobius Level-2 plasmids, Loop pEven plasmids; (ii) MoClo plasmids; or (i) or (ii).

In some embodiments, the present disclosure provides a gene stacking system comprising: (a) a first donor vector for delivering target DNA inserts; (b) a second donor vector for delivering target DNA inserts; (c) an acceptor vector configured to contain up to about 300 kb of target DNA inserts; and (d) an engineered bacterial cell comprising the acceptor vector, wherein the engineered bacterial cell is configured to express at least one recombinase. In some embodiments, the first donor vector is configured to deliver at least a first target DNA insert to the acceptor vector and the second donor vector is configured to deliver at least a second target DNA insert to the acceptor vector. In some embodiments, the first donor vector is configured to deliver multiple of the same or different target DNA inserts to the acceptor vector via one or more rounds of integration and the second donor vector is configured to deliver multiple of the same or different target DNA inserts to the acceptor vector via one or more rounds of integration. In some embodiments, expression of the at least one recombinase by the engineered bacterial cell is inducible. In some embodiments, the engineered bacterial cell further comprises a recombineering system. In some embodiments, the recombineering system enables editing of the target DNA inserts and/or the acceptor vector. In some embodiments, the editing comprises: (i) single nucleotide editing; (ii) insertion of DNA fragments up to 300 kb; (iii) deletion of any parts of the acceptor vector; and/or (iv) replacement of any-size fragment of the acceptor vector with up to 300 kb of replacement DNA. In some embodiments, the recombineering system comprises heat-shock-inducible lambda Red proteins, said proteins comprising Exo, Beta and Gam. In some embodiments, the first donor vector comprises attB recombination donor sites, the second donor vector comprises attP recombination donor sites, and the acceptor vector comprises an attP recombination acceptor site. In some embodiments, the first donor vector comprises attP recombination donor sites, the second donor vector comprises attB recombination donor sites, and the acceptor vector comprises an attB recombination acceptor site. In some embodiments, expression of a first recombinase induces integration of the entire first donor vector into the acceptor vector. In some embodiments, expression of the first recombinase induces integration of the entire second donor vector into the acceptor vector. In some embodiments, the first recombinase is expressed by the engineered bacterial cell in an inducible manner. In some embodiments, the first recombinase is a rhamnose-inducible PhiC31 recombinase. In some embodiments, the first donor vector comprises a first recombination donor site that is compatible with a first recombination acceptor site, wherein said first recombination acceptor site is present in the acceptor vector prior to the integration of the entire first donor vector into the acceptor vector; and the second donor vector comprises a second recombination donor site that is compatible with a second recombination acceptor site, wherein said second recombination acceptor site is present in the acceptor vector prior to the integration of the entire second donor vector into the acceptor vector. In some embodiments, each of the recombination donor sites and each of the recombination acceptor sites are recognized by the same recombinase. In some embodiments, each of the recombination donor sites and each of the recombination acceptor sites are recognized by PhiC31 recombinase. In some embodiments, the first donor vector comprises a first donor vector insert portion and a first donor vector backbone portion and expression of a second recombinase after the first donor vector has been integrated into the acceptor vector induces removal of the first donor vector backbone portion from the acceptor vector; and the second donor vector comprises a second donor vector insert portion and a second donor vector backbone portion and expression of a second recombinase after the second donor vector has been integrated into the acceptor vector induces removal of the second donor vector backbone portion from the acceptor vector. In some embodiments, the second recombinase is expressed by the engineered bacterial cell in an inducible manner. In some embodiments, the second recombinase is an arabinose-inducible FLP recombinase. In some embodiments, the first donor vector comprises a first excision site; the second donor vector comprises a second excision site; and the acceptor vector comprises a third excision site. In some embodiments, the first, second, and third, excision sites are recognized by the same recombinase. In some embodiments, the first, second, and third, excision sites are FLP recombinase sites (also referred to herein as “FRT sites”). In some embodiments, the first donor vector and the second donor vector each comprise a SacB gene. In some embodiments, the first donor vector, the second donor vector and the acceptor vector each comprise at least one selection marker. In some embodiments, the selection marker is an antibiotic resistance gene. In some embodiments, the first donor vector, the second donor vector and the acceptor vector each comprise a different selection marker. In some embodiments, the gene stacking system is compatible with Type IIS restriction enzyme-based cloning technologies in plants. In some embodiments, the first donor vector and/or the second donor vector are configured to incorporate target DNA inserts from: (i) Golden Gate vectors, GoldenBraid plasmids, Mobius Level-2 plasmids, and Loop pEven plasmids; or (ii) MoClo plasmids.

In some embodiments, the present disclosure provides a gene stacking system comprising: at least one donor vector comprising at least one target DNA insert flanked by recombination donor sites; at least one acceptor vector comprising recombination acceptor sites compatible with the recombination donor sites of the at least one donor vector, wherein the at least one acceptor vector is configured to contain up to about 300 kb of target DNA insert; and an engineered bacterial cell comprising the at least one acceptor vector, wherein the engineered bacteria cell is configured to express at least one recombinase. In some embodiments, expression of the at least one recombinase by the engineered bacterial cell is inducible. In some embodiments, the engineered bacterial cell further comprises a recombineering system. In some embodiments, the at least one donor vector is used for delivering multiple different target DNA inserts to the acceptor vector. In some embodiments, the gene stacking system is compatible with Type IIS restriction enzyme-based cloning technologies in plants. In some embodiments, the gene stacking system is compatible with Agrobacterium-mediated transformation.

In some embodiments, the provided gene stacking systems are compatible with all major synthetic biology cloning methods (e.g., Golden Gate, GoldenBraid, MoClo, Mobius, and Loop) that rely on Type IIS restriction enzymes and follow standard syntax rules. In some embodiments, use of transformable BAC backbone in the construction of the acceptor vector of the gene stacking system brings the upper DNA cargo size limit to 300 kb. In some embodiments, the gene stacking systems enable editing of constructs post-assembly, offering an unprecedented level of flexibility.

In some embodiments, the present disclosure provides an engineered bacterial cell comprising: (a) a recombincering system; and (b) an acceptor vector configured to contain up to about 300 kb of target DNA inserts. In some embodiments, the bacterial cell is a lambda Red recombineering-competent E. coli. In some embodiments, the bacterial cell expresses: (i) exo, beta and gam genes in a heat-shock inducible manner; and (ii) a FLP recombinase in an arabinose-inducible manner. In some embodiments, the bacterial cell further expresses an inducible recombinase configured to mediate selective recombination between vectors in the bacterial cell. In some embodiments, the bacterial cell further expresses an inducible PhiC3/recombinase. In some embodiments, the PhiC31 is expressed in a rhamnose-inducible manner. In some embodiments, the acceptor vector comprises sites recognized by PhiC31 recombinase and FLP recombinase.

In some embodiments, the present disclosure provides a method of integrating target DNA inserts into an acceptor vector, said method comprising a first round of integration comprising: (a) introducing a first donor vector into a gene stacking-competent bacterial cell carrying an acceptor vector, wherein the acceptor vector is configured to contain up to 300 kb of target DNA inserts and wherein the first donor vector comprises a first donor vector backbone portion and a first donor vector insert portion; (b) inducing integration of the first donor vector into the acceptor vector; and (c) removing the first donor vector backbone portion from the acceptor vector; wherein, the first donor vector insert portion is configured to deliver target DNA inserts to the acceptor vector. In some embodiments, the method further comprises a second round of integration comprising: (d) introducing a second donor vector into the gene stacking-competent bacterial cell, wherein the second donor vector comprises a second donor vector backbone portion and a second donor vector insert portion; (e) inducing integration of the second donor vector into the acceptor vector; and (f) removing the second donor vector backbone portion from the acceptor vector, wherein the second donor vector insert portion is configured to deliver target DNA inserts to the acceptor vector. In some embodiments, the method further comprises additional rounds of integration, wherein each additional round of integration comprises: (g) introducing the first donor vector into the gene stacking-competent bacterial cell, (h) inducing integration of the first donor vector into the acceptor vector; and (i) removing the first donor vector backbone portion from the acceptor vector; or (j) introducing the second donor vector into the gene stacking-competent bacterial cell, (k) inducing integration of the second donor vector into the acceptor vector; and (l) removing the second donor vector backbone portion from the acceptor vector; wherein the method comprises alternating between parts (g)-(i) and parts (j)-(l); and wherein each additional round of integration delivers target DNA inserts to the acceptor vector. In some embodiments, the first donor vector is configured to deliver multiple of the same or different target DNA inserts to the acceptor vector via one or more rounds of integration. In some embodiments, the second donor vector is configured to deliver multiple of the same or different target DNA inserts to the acceptor vector via one or more rounds of integration. In some embodiments, inducing integration of the first donor vector comprises induction of recombination between the first donor vector and the acceptor vector. In some embodiments, inducing integration of the second donor vector comprises induction of recombination between the second donor vector and the acceptor vector. In some embodiments, removing the first donor vector backbone portion from the acceptor vector comprises excision of the first donor backbone portion from the acceptor vector via homologous recombination mediated by FLP recombinase. In some embodiments, removing the second donor vector backbone portion from the acceptor vector comprises excision of the second donor backbone portion from the acceptor vector via homologous recombination mediated by FLP recombinasc. In some embodiments, the method further comprises editing of the integrated DNA inserts and/or acceptor vector. In some embodiments, the editing comprises: (i) single nucleotide editing; (ii) insertion of DNA fragments up to 300 kb; (iii) deletion of any parts of the acceptor vector; and/or (iv) replacement of any-size fragment of the acceptor vector with up to 300 kb of replacement DNA. In some embodiments, the editing comprises using a recombincering system. In some embodiments, the gene stacking-competent bacterial cell comprises a recombineering system. In some embodiments, the recombincering system comprises heat-shock-inducible lambda Red proteins, said proteins comprising Exo, Beta and Gam. In some embodiments, the method further comprises delivering a part of the acceptor vector comprising the target DNA inserts to a target cell. In some embodiments, the target cell is a plant cell. In some embodiments, delivering a part of the acceptor vector to the target cell comprises Agrobacterium-mediated transformation.

### BRIEF DESCRIPTION OF THE DRAWINGS

FIG. 1: Schematic illustrating DNA cargo capacity of standard vectors and the gene stacking system provided herein. Popular synthetic biology gene assembly systems such as the classical GoldenBraid technology, have limited DNA cargo capacity.

FIG. 2: Schematic depicting illustrative components of a gene stacking system provided herein.

FIG. 3: Sequence of PhiC31 (605 aa version, highlighted in blue) with partial Shine-Dalgarno (SD) sequence (ggagg) and spacer sequence (acaatc) at its 5′ end. After integration into the E. coli SW105 genome, the partial SD sequence and the stop codon (TAA) of the upstream gene will form the optimal SD sequence. The spacer sequence is described to typically vary from 5 to 13 nt.

FIGS. 4A-4B: Schematic depicting engineering of acceptor vector. A fragment containing an attP^{TT} and FRT sites flanked by ˜40 nt arms of homology to the JATY vector sequence (FIG. 4A) was generated by PCR and used for homologous recombination into the JATY clone by recombineering. The resulting T-DNA region in the acceptor vector (FIG. 4B) contains an attP^{TT} and a FRT site flanked by ˜50 nt of original T-DNA sequences. These two flanking regions can be used as docking sites for further vector modification if needed.

FIGS. 5A-5B: Schematic depicting engineering of donor vectors. Two alpha vectors and two omega vectors that are components of the standard GoldenBraid molecular cloning system (FIG. 5A) were modified to produce two sets of donor vectors shown in dashed rectangles (FIG. 5B). Each set consists of Donor 1 (D1, DI) and Donor 2 (D2, DII) with different antibiotic genes so that there is one Donor 1 in alpha and one Donor 1 in omega vector. For each vector, an att site was inserted upstream of lacZ selectable marker (between LB marked by the black line and BsaI/BsmBI sites marked by the red line). This att will be used for integration. Downstream of lacZ, the second att site and an FRT site were inserted between BsaI/BsmBI sites (red line) and RB (black line). The FRT is needed for the removal of the donor vector backbone, while the second att is necessary for the integration of the second donor vector. In addition, for alpha vectors, Kan was replaced by Amp to make these donor vectors compatible with the Kan-resistant acceptor vector. Furthermore, partial Shine-Dalgarno sequence (GGAGG) and spacer sequence (ACAATC) followed by SacB was inserted downstream of the stop codon of the Ampicillin (Amp) or Spectinomycin (Spec) resistance gene to allow for vector counter-selection in the presence of sucrose (sucrose in the media is toxic to SacB-containing clones).

FIG. 6: Schematic depicting engineering of Alpha1-D2 donor vector. To make Alpha1-D2, PCR fragments containing attP^{CC} and attP^{TT}-FRT were inserted between EcoRI and BsaI, BsaI and EcoRI sites, respectively, in the original GoldenBraid pDGB3alpha1 vector. The att site's central dinucleotide is underlined in the schematic. In addition, the original Kan selectable marker gene was replaced with Amp-SacB to enable positive and negative selection.

FIG. 7: Sequence of domesticated ampicillin (Amp) resistance and SacB genes. The domesticated ampicillin (Amp) resistance (marked in gray) and SacB (marked in red) genes were linked into one operon by the optimal SD sequence and a spacer sequence, which was used to replace the original kanamycin (Kan) resistance gene. The start and stop codons of Amp and SacB genes are underlined.

FIG. 8: Schematic depicting engineering of Omega1-D1 donor vector. To make Omega1-D1, PCR fragments containing attB^{TT} and attB^{CC}-FRT were inserted between BamHI and BsaI, BsaI and BamHI sites, respectively, in the original GoldenBraid pDGB3omega1 vector. The att site's central dinucleotide is underlined in the schematic. In addition, partial Shine-Dalgarno sequence (GGAGG) and spacer sequence (ACAATC) followed by SacB gene was inserted downstream of the stop codon of the Spec marker to enable counter-selection.

FIG. 9: Sequence of domesticated SacB. Partial Shine-Dalgarno sequence (GGAGG) and spacer sequence (ACAATC) followed by SacB (highlighted in orange) were inserted downstream of the stop codon of the spectinomycin (Spec) resistance gene. The start and stop codons of SacB are underlined.

FIG. 10: Schematic depicting engineering of Alpha2-D1 donor vector. To generate Alpha2-D1, PCR fragments containing attB^{TT} and attB^{CC}-FRT were inserted between HindIII and BsmBI, BsmBI and HindIII sites, respectively, in the original GoldenBraid pDGB3alpha2 vector. In addition, the original Kan gene was replaced with Amp-SacB positive-negative dual selectable marker.

FIG. 11: Schematic depicting engineering of Omega2-D2 donor vector. To make Omega2-D2, PCR fragments containing attp^{CC} and attP^{TT}-FRT were inserted into the two EcoRV sites (that flank the BsaI sites and lacZ) in the original GoldenBraid pDGB3omega2 vector. This subcloning regenerates intact EcoRV sites upstream of attp^{CC} and downstream of attP^{TT}-FRT in their original positions. In addition, partial Shine-Dalgarno sequence (GGAGG) and spacer sequence (ACAATC) followed by SacB was inserted downstream of the stop codon of Spec.

FIG. 12: Schematic depicting engineering of Alpha1-D2 MoClo donor vector. To make Alpha1-D2 MoClo, TGCC was inserted and GGGA was used to replace the original GoldenBraid grammar, cgct, so that when the vector is digested by BsaI, it can accept DNA fragment(s) with grammar TGCC and GGGA from MoClo. Importantly, the original GoldenBraid grammar codes, ggag and gtca, in Alpha1 were kept intact so that the construct can be assembled according to GoldenBraid system. In addition, partial Shine-Dalgarno sequence (GGAGG) and spacer sequence (ACAATC) followed by SacB was inserted downstream of the stop codon of Amp.

FIG. 13: Schematic depicting engineering of Alpha2-D1 MoClo donor vector. To make Alpha2-D1 MoClo, MoClo grammar codes TGCC and GGGA were introduced for BsaI digestion, and GoldenBraid grammar gtca and cgct were kept for BsmBI digestion for next assembly.

FIG. 14: Schematic depicting integration of 8 TUs (23.167 kb) into the acceptor vector.

FIG. 15: Colony PCR was performed on colonies that grew on LB+Kan plates after the dual sugar treatment. Two pairs of primers were used to test 20 randomly picked colonies, and 15 colonies (out of 20) were found to be positive by PCR. Thus, the integration efficiency of an 8 TU module from the donor to the acceptor was 75%.

FIG. 16: Compatibility of the DASH assembly and gene stacking system with other common DNA assembly platforms. Like all other systems based on Golden Gate, DASH can be utilized to assemble domesticated DNA parts into transcriptional units (TUs). Once these TUs are in a DASH vector, they can be further combined into modules (MDs) in a typical reiterative binary assembly procedure characteristic of the GoldenBraid (GB) system as long as all DNA parts have been domesticated for both BsaI and BsmBI. Thus, the DNA assembly components of the DASH system are fully compatible with GoldenBraid parts, TUs, and MDs. Parts domesticated for other systems that also use BsaI but not BsmBI, such as Mobius, Loop, and MoClo, can be assembled into TUs using the pDASH alpha vectors to facilitate their subsequent stacking in the pDASH-AIK destination vector. Likewise, DNA constructs in Level Even of Loop, Level 2 of Mobius, and Level 1 of Star-Stop can also be directly transferred to a pDASH alpha vector using the BsaI enzyme. The system's compatibility can be further enhanced by generating shuttle vectors such as the pDASHa MoClo, which allows the transfer of DNA constructs from MoClo Level M vector into the DASH system. Once a DNA construct is in one of the pDASH alpha vectors, it can be directly used in a stacking experiment and transferred to the pDASH-AIK destination vector or, if lacking the BsmBI recognition sites, combined with other DNA constructs already in the DASH system. Thus, the DASH system enables the stacking of DNA constructs created using various assembly method platforms and overcomes the size limit for GoldenBraid, Loop, Mobius, MoClo, and Start-Stop systems. Moreover, recombineering can be utilized to create any post-assembly modifications after the DNA constructs are stacked in the pDASH-AIK destination vector.

FIGS. 17A-17B: Key components of the DASH system. (A) DASH system is composed of three essential elements. 1) The CZ105a strain of E. coli carries in its genome the temperature-inducible lambda-Red recombincering system, an arabinose-inducible FLP recombinase, and a rhamnose-inducible PhiC31 integrase (A, top panel). 2) Four donor vectors derived from the pDGB GoldenBraid plasmids that incorporate the recognition sites for the PhiC31 integrase and the FLP recombinase. Using two sets of orthogonal attB-attP sites allows the reiterative insertion of the donor vectors into the acceptor vector following a D1→DII→D1→DII, etc. arrangement. The presence of FRT sites facilitates the removal of the donor vector backbone upon integration (A, middle panel). 3) A single acceptor vector derived from the TAC PYLTAC17 vector but containing an attP^{TT} site to facilitate the integration of the first donor vector and the FRT site to allow for the elimination of the donor vector backbone (A, bottom panel). (B) To improve the system's compatibility and aid in removing the donor vector-derived sequences, two additional sets of vectors were created. The MoClo shuttle donor vectors are modifications of the standard pDASH alpha vectors, designed so that after digestion with the BsaI enzyme, the resulting overhangs are compatible with those used in the MoClo Level M vectors (B, top panel). In addition, the SacB counter-selectable marker gene was added to all four pDASH donor vectors to facilitate their elimination from E. coli after the integration of their cargo into the acceptor vector (B, middle panel). The second acceptor vector with the attB^{CC} site was also produced to facilitate the initiation of the stacking process with the DII donor vectors (B, bottom panel). Thin black arrows mark the recognition sites for the BsmBI enzyme, while the thin red arrows mark the recognition sequences for the BsaI enzyme. Black sequences inside of rectangles indicate the overhangs created after the BsmBI digestion, while the red sequences in the rectangles show the overhangs generated by the BsaI enzyme. The different attP, attB and FRT sites are depicted as arrow heads of different colors. The different selectable marker genes (Kan, Amp, Spec, LacZ and SacB) are shown as arrows of different colors. The vertical black lines represent the left and right borders of the T-DNA.

FIGS. 18A-18B: DASH donor vector assembly works seamlessly with GoldenBraid components, but it becomes inefficient for constructs larger than approximately 25 kb. (A) Transcription units (right arrows, drawing not to scale) in GoldenBraid (GB) consist of the following sequences: 35S-MCP-VPR-Tnos (GB α1-TU1), 35S-dCas9_{spy}-EDLL-Tnos (GB α2-TU2), AtU6p-sgRNA_{Spy(SIDFR)}-MS2-A1U6t (GB α1-TU3), SIDFRP_{Spy(SIDFR)}-3xYpet-Term35S #0 (GB α2-TU4), 35S-dCas9_{Sth}-EDLL-Term35 #0 (GB α1-TU5), Kan (GB0184) (GB α2-TU6), AtU6p-sgRNA_{Sth(ADH1)}-MS2-A1U6t (GB α1-TU7), SIDRFp_{Sth(ADH1)}-mCherry-Term35S #0 (GB α2-TU8), FAST seed fluorescent marker (GB α1-TU9), and Basta (GB0023) (GB α2-TU10). These transcriptional units were assembled in the typical GoldenBraid pairwise fashion, as indicated by the horizontal brackets. The names of the modules (MDs) resulting from these pairwise assemblies are also shown. (B) The size of the different modules and the corresponding assembly efficiencies are shown.

FIG. 19: The DASH system can efficiently generate large gene stacks of over 90 kb. Schematic representation of the seven consecutive integration/excision reactions used to stack 35 transcriptional units in the final destination plasmid. The PhiC31 recombination sites involved in each stacking cycle between a donor and acceptor vector are shown, as well as the architectures of the resulting plasmids. Transcriptional units are shown as broad arrows, while large modules are shown as large orange arrows containing transcriptional unit arrows. Triangles depict recombination sites for either PhiC31 or FLP. The cargo size of each of the vectors, as well as the efficiency of each integration/excision cycle, are also indicated. ND, not determined.

FIGS. 20A-20F: Functional characterization of the 116 kb construct (>97 kb T-DNA) generated with the DASH system. (A) Schematic representation of the final >97 kb cassette containing four repeats of Module 7 interspaced by the lacZ, FAST and sfGFP transcriptional units (pDASH-AIK-Big). Primers used to test the presence of the different components are shown. (B) Activity of the LacZ and sfGFP markers in bacteria harboring the pDASH-AIK-Big construct (upper) compared with the control (the acceptor vector only, pDASH-AIK) (lower). (C) Activity of the YPet and mCherry fluorescent proteins in N. benthamiana leaves transfected with Agrobacterium containing the pDASH-AIK-Big construct (enlarged cells shown in an inset). Expression of these two fluorescent proteins requires the activity of seven out of the eight transcriptional units comprising module 7. (D) Activity of the FAST reporter in seeds of Arabidopsis TO plants. (E) Activity of the sfGFP fluorescence and Ampicillin-resistance markers in bacteria harboring the pDASH-AIK-Big construct before (top) and after (bottom) the recombincering-based post-assembly replacement of sfGFP with the Ampicillin-resistance gene. (F) Expression of the Ypet and mCherry in T1 Arabidopsis seedlings carrying the pDASH-AIK-Big T-DNA construct.

FIGS. 21A-21B: Genomic modifications in the DASH CZ105 strains. (A) Schematic representation of three strains of E. coli. The relevant genetic components of the original E. coli strain SW105, including a defective, temperature-sensitive λ prophage harboring three Δ Red genes, exo, beta, and gam, for homologous recombination in the genome, are shown. In addition, the genome of the SW105 strain also carries an L-arabinose-inducible FLP gene cassette. To generate the DASH strain CZ105a, the phage-derived PhiC3/integrase coding sequence and a ribosome binding site were inserted into the rhamnose-catabolismrhaBAD operon just downstream of the rhaA coding sequence. In the CZ105b strain, the same sequences were inserted just downstream of the transporter gene rhaT. (B) The Sanger sequencing chromatograms of the junctions between the PhiC31 and the flanking sequencing in the E. coli genome are shown.

FIGS. 22A-22B: Functional analysis of the PhiC31 integrase and FLP recombinase activities in CZ105a and CZ105b cells. (A) Schematic representation of the pDASH-AIK acceptor vector before (left panel) and after (right panel) PhiC31-mediated integration and subsequent FLP-mediated excision of pDASH-DI-ω1 vector sequences. (B) PCR amplification of 20 CZ105a (left panels) and 10 CZ105b (right panel) colonies using the primers P27 and P28 flanking the recombination sites in the pDASH-AIK vector. The larger band (**) corresponds to plasmids where both the integration and excision have taken place, while the smaller band (*) corresponds to plasmids where no insertion has occurred. Whole plasmid sequencing of colony 30 indicates that weaker bands correspond to colonies where only a small fraction of the cells have undergone the integration and excision process. On the other hand, Sanger sequencing of the junction sites of the DNA from lane 7 indicates that strong bands correspond to colonies where integration and excision have both occurred. Letters M, D, and A indicate marker, donor, and acceptor DNA controls, respectively. Lane C in the CZ105a gels corresponds to colony 30 in gel CZ105b.

FIGS. 23A-23B: Fidelity of two consecutive rounds of PhiC31/FLP-mediated integration/excision reactions. (A) Schematic representation of the PhiC31/FLP-mediated integration and excision of pDASH-AIK and pDASH-DI-ω1 to generate the pDASH-AIK-lacZ construct, followed by a second round of integration and excision between the plasmids pDASH-AIK-lacZ and pDASH-DII-ω2 to produce the pDASH-AIK-lacZ². (B) The chromatograms from the Sanger sequencing that correspond to the junction sites of the clones resulting from each integration/excision reaction, highlighted in red boxes in panel A, are displayed in panel B. Key parts of the sequences, such as recombination sites and marker genes, are annotated.

FIGS. 24A-24F: Example of the assembly efficiency estimation for large modules. (A) Schematic representation of module 7 obtained by assembling module 5 and module 6, as indicated by the brackets. The transcriptional units are depicted as large arrows, while the relative positions of the primers used for colony PCRs are marked with small black arrows. (B) The efficiency of assembling modules pDASH-DII-α1-MD5 and pDASH-DI-α2-MD6 to generate the 23 kb pDASH-DI-ω1-MD7 was determined by first examining the number of LacZ-positive (blue) colonies (not recombinant) and LacZ-negative (white) colonies (potential recombinants). Of the 51 colonies obtained, 14 were white, and the rest were blue. Colony PCR of the 14 white colonies using the diagnostic primers mCherry f and pDGB3 r indicates that 9 out of the 14 colonies contained sequences from Module 6 (left panel). PCRs with the primers MF_Ypet3′_F and StdCas9 D9Ar indicate that all these nine colonies contain sequences from Module 5 (right panel). (C) Diagnostic restriction digest of the plasmids corresponding to the nine PCR-positive colonies using two different enzymes, EcoRI or XhoI, indicates that seven of the nine colonies contain Module 7. Sequencing of the final ˜ 97 kb construct confirmed the fidelity of the assembly of Module 7. The incorrect clones based on the digestion pattern, 5 and 7, are marked with light blue numbers. M indicates a DNA marker, while “+” is a positive control. VD indicates virtual digestion. D) Schematic representation of module 9 attempted by assembling modules 7 and 8 together. The transcriptional units are depicted as large arrows, while the relative positions of the primers used for the colony PCRs are marked with small black arrows. (E) White colonies from module 9 assembly were examined by PCR using the primers Term0 F and FAST r2. Weak amplification was observed in 3 out of the 25 colonies examined (11 are shown). (F) Diagnostic restriction digest of the plasmids corresponding to three PCR-positive colonies with the enzyme NsiI indicates that none contain the intact module 9 sequence. Virtual digestion of module 7 (MD7 VD) and module 9 (MD9 VD) are shown. The green box highlights two diagnostic bands (2191 and 1994 bp in size) that should be identical in both MD7 and MD9 but are only seen in the MD7 control digestion (MD7 label) but not in any of the MD9 DNAs (MD9 label).

FIG. 25: Simple user guide for the DASH gene stacking system. A brief overview of the steps involved in a typical gene stacking experiment using the DASH system is provided. The estimated hands-on time for each step and the total duration of the process are indicated. The guide assumes that the user already has the DASH destination vector in the CZ105a cells and that the transcriptional unit or module to be stacked is already present in a DASH donor vector.

### DETAILED DESCRIPTION

Embodiments of the present disclosure provide systems and tools for implementing synthetic biology methods in plants. Existing technologies have several drawbacks, including the cargo size limitation of existing vector systems and inability to edit constructs after they have been assembled.

The development of DNA assembly methods, including Gibson assembly, Gateway cloning and In-Fusion, among others, has greatly facilitated the widespread adoption of synthetic biology approaches. Among all DNA assembly methods, Golden Gate and its variants, such as GoldenBraid, MoClo, Mobius, Loop, and others, have gained widespread popularity due to their efficiency in combining multiple DNA fragments in a single-tube enzymatic restriction/ligation reaction. By taking advantage of the Type IIS restriction enzymes, which cut DNA outside of their recognition sequence, Golden Gate-based cloning approaches enable the assembly of multiple DNA fragments of almost any sequence composition using a very limited number of restriction enzymes, typically from one to three. This makes these technologies particularly useful for precise and flexible manipulation of standardized DNA parts.

Despite the advantages of Golden Gate-based systems mentioned above, they also present certain challenges. For instance, a domestication step is often necessary to eliminate internal recognition sites of the enzymes used for DNA assembly, creating potentially undesired scars. The problems and inconveniences associated with the domestication process are accentuated when two or more Type IIS enzymes are employed to allow for additional DNA assembly cycles required when combining multiple transcriptional units. By using DNA methylases that target sequences partially overlapping with the recognition sites of a Type IIS enzyme, the MetClo system can utilize a single Type IIS restriction enzyme even during multi-step assemblies. This is achieved by engineering the flanking sequences of some of the Type IIS recognition sites to overlap with the target sequence of a specific DNA methylase. These sites' methylation status can then be switched on or off by propagating the MetClo plasmids in E. coli strains expressing or not the corresponding methylase. Although the strategy adopted by MetClo bypasses to some degree the need for domesticating DNA fragments for multiple Type IIS enzymes, it has the inconvenience of switching between different E. coli strains.

In addition to the domestication process, adopting standardized syntax for the exchange of DNA parts also represents a source of scars, as each DNA part type (promoter, coding sequence, terminator, etc.) is flanked by an agreed-upon four-nucleotide fixed sequence aka a “part's code”. In this case, the scars created by following the grammar rules are located at each of the DNA part's junctions. To reduce the potential impact of these junction scars, the Start-Stop Assembly technology uses the Type II enzyme with three-instead of a four-nucleotide overhang at each part's flanks. Even though this approach can eliminate the scars at the start and stop codons, scars are still formed at the junction of the other standardized DNA parts. Thus, none of the Golden Gate-based cloning systems can be considered entirely scar-free.

In addition to the use of common three- or four-nucleotide codes, the compatibility between the different Golden Gate-based systems also depends on the Type IIS enzymes used. Thus, for example, even though the Type IIS restriction enzyme, BsaI, is often used to generate transcriptional units, its use is not universal among Golden Gate-based systems. Moreover, when selecting a second or third Type IIS restriction enzyme to enable additional cycles of DNA assembly, the choice of enzymes across Golden Gate cloning systems becomes even more inconsistent. For example, GoldenBraid 3.0 uses BsaI and BsmBI, MoClo leverages BsaI and BpiI, and Loop relies on BsaI and SapI.

Another common challenge of most Golden Gate-based systems is the lack of efficient post-assembly modification capabilities. This means that any modification, such as replacing a promoter sequence to alter the expression levels of a gene in a multigene construct, necessitates creating a new transcriptional unit with the alternative promoter and reassembling the entire genetic circuit, often from scratch. This represents an important handicap for the “design-build-test-learn” (DBTL) cycle characteristic of synthetic biology approaches, justifying the interest in developing efficient and flexible post-assembly-modifiable, Type IIS-compatible cloning and assembly systems. Homology-based recombination approaches such as recombineering are especially suited for this type of post-assembly modifications as they allow for the insertion, deletion, and replacement of basically any sequence in a plasmid by using the recombincering molecular machinery engineered into E. coli strains such as SW105. In this recombineering strain, three lambda-Red phage genes, exo, beta, and gam, are integrated into the bacterial genome and expressed under the strict control of the temperature-inducible λpL/pR-c1857 system. The induction of these three proteins leads to very high levels of recombination between the linear donor DNA and its target, regardless of whether the target gene is located in a plasmid or within the bacterial genome. Importantly, this recombination is directed by as little as 40 nt of homology arms, i.e., sequences with 100% identity between the flanks of the donor molecule and the target DNA.

One final limitation of Golden Gate-based systems is the substantial decrease in assembly efficiency as the size of DNA fragments increases, which is further compounded by the typically limited cargo capacity of most plasmids used in these platforms. In fact, the cargo capacity of the plasmid typically used in Golden Gate-based systems rarely exceeds 25-50 kb. The MetClo system discussed above utilizes a high-capacity vector with a p15a or F replication origin that confers a low plasmid copy number and an increased cargo capacity. However, simply increasing the cargo capacity of a plasmid does not address the limitations of the Golden Gate-based systems to operate efficiently with large DNA molecules, a challenge still observed with the high-capacity MetClo system. Therefore, to effectively handle larger DNA fragments, Golden Gate-based systems need not only to increase the cargo capacity of the plasmids used, but also to improve efficiency in assembling these larger DNA fragments. One approach to addressing the low assembly efficiency of large DNA fragments is using site-specific recombinases such as Cre, FLP, PhiC31, or Bxbl. These enzymes efficiently catalyze the integration and excision of large circular DNA molecules, overcoming the limitations of other in vivo recombination-based systems, such as recombineering and jStack, which are constrained by the low efficiency of introducing large linear DNA molecules into cells.

One elegant example of the in vivo use of site-specific recombinases for the generation of plant transformation-ready constructs is the GAANTRY (gene assembly in Agrobacterium by nucleic acid transfer using recombinase technology) gene stacking system. This system consists of an Agrobacterium strain (ArPORT1) carrying a disarmed virulent pRi plasmid engineered with a recombinase P site, two donor plasmids and two helper vectors with recognition sites and the corresponding recombinases, respectively. The transient expression of the A118 recombinase results in the integration of the whole B donor vector into the P site of the pRi plasmid, whereas the ParA recombinase catalyzes the excision of the backbone sequences of the inserted B donor vector. The DNA cargo integration process can be repeated many times by alternating the transformation of the B donor plus helper in one cycle and the P donor plus helper vectors in the next to generate large multigene DNA constructs. Importantly, the efficiency of the integration process is close to 100%, and the resulting Agrobacterium strain can be directly used to effectively transform plants. The use of the Agrobacterium non-binary pRi acceptor plasmid makes any post-assembly modifications challenging when using this system. In fact, the sequential nature of the gene stacking process necessitates that introducing a modification in the final construct requires generating not only a new DNA part but also repeating all the corresponding stacking steps.

Another exciting example of a highly efficient, in vivo recombinase-based stacking system is the TGSII system (TransGene Stacking II) that utilizes the recombinase Cre and a combination of wild-type and self-compatible loxP mutant sites to carry out the plasmid integration and backbone excision reactions. In contrast with the GAANTRY system, however, the integration of a second cargo DNA fragment requires additional steps, including the extraction, purification, and digestion of the resulting plasmid mixture with the I-SceI or PI-SceI homing endonuclease in order to eliminate undesired plasmid species and isolate the intended acceptor vector-cargo DNA complex. The addition of these in vitro steps makes this method somewhat more cumbersome than the GAANTRY system, where the acceptor vector remains in the Agrobacterium cells throughout the stacking process. Importantly, like the GAANTRY system, the TGSII is not fully integrated with the Golden Gate-based system and, by itself, lacks post-assembly modification capabilities.

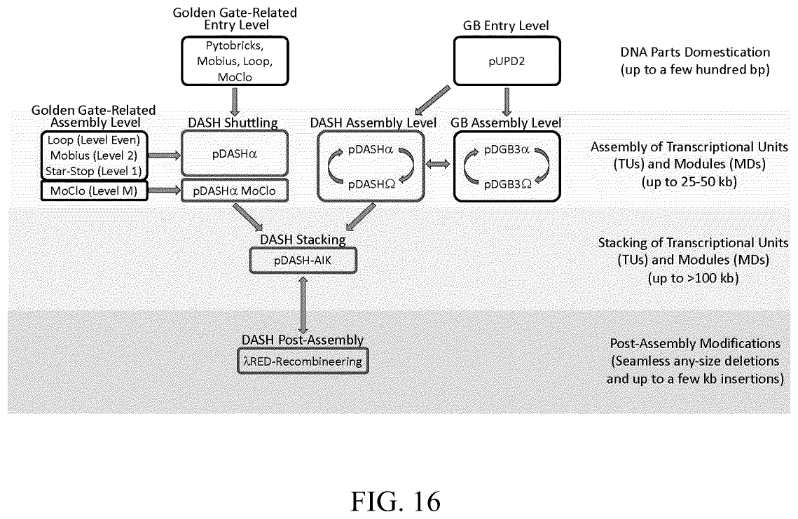

To overcome some of the technical barriers often found in the Golden Gate-based systems, embodiments of the present disclosure include an innovative assembly and stacking system named DASH (DNA assembly and stacking hybrid). This system takes advantage of the reference restriction enzyme, BsaI (so that the donor vectors can accept the standardized DNA parts and assembled transcription units from various Golden Gate-based cloning systems) and combines the benefits of the GoldenBraid reiterative assembly capabilities, the high cargo capacity of the transformation-competent bacterial artificial chromosome (TAC) single-copy binary vector pYLTAC17, the high in vivo integration/excision efficiency of the PhiC31 and FLP recombinases, and the precision of the genome editing recombineering technology at introducing any sequence changes in DNA molecules of any size. Importantly, the gene stacking capabilities of the DASH platform can be used to overcome the size limitations not only of DNA constructs assembled using the DASH and GoldenBraid systems but also those of transcriptional units and modules generated in other Golden Gate-based cloning systems that use the BsaI enzyme in their assembly, such as Loop, MoClo, Mobius, and Start-Stop (FIG. 16). Finally, the TAC acceptor vector in DASH has the capacity to stably maintain a large DNA fragment of up to 300 kb while the recombincering machinery is available for post-assembly modification, including insertion, deletion, and replacement of any sequence precisely and efficiently.

Notably, the DASH system has been designed for high efficiency and experimental simplicity. Thus, all the initial assembly steps of combining DNA parts to generate transcriptional units and small multigenic constructs take advantage of the single tube restriction/ligation reaction characteristic of Golden Gate-based systems. Single genes or multigenic constructs generated using the DASH system or a variety of Golden Gate-based platforms can easily and efficiently be stacked into a high-capacity acceptor vector (FIG. 16). This is achieved by simply electroporating the cargo-carrying DASH donor vector into the SW105-derived E. coli strain, CZ105, containing the pDASH-AIK acceptor vector and activating different recombinases in vivo by growing the cells in the presence of different sugar inducers (rhamnose or arabinose). The selection of the cargo-containing acceptor vector is also done in vivo using antibiotics, while the removal of the donor plasmid and the corresponding cargo-less backbone can be facilitated by the SacB contra-selectable marker. Finally, the temperature-inducible recombineering system integrated into the CZ105 cells further simplifies the post-assembly modification process, minimizing the need for the in vitro manipulation of large DNA molecules (FIG. 16). The practicality of the DASH system was demonstrated by generating a 35-gene cassette with a total DNA insert of over 97 kb and validating the functionality of these large constructs in Nicotiana benthamiana transient expression assays and stable Arabidopsis thaliana transformants. Finally, the post-assembly capabilities of the system was also confirmed by replacing the sfGFP with that of RPSL-Amp cassette in the final ˜97 kb construct cargo by leveraging the recombineering feature of the DASH system.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

As used herein, the term “about” refers to plus or minus up to 10% of the value it modifies (rounded up to the nearest whole number if the value is not sub-dividable, such as a number of molecules or nucleotides). The term “about,” when modifying the quantity (e.g., mg) of a substance or composition, a parameter of a substance or composition or a parameter used in characterizing a step in a method, or the like, refers to variation in the numerical quantity that can occur. Such variation can occur through typical measuring, handling, and sampling procedures involved in the preparation, characterization and/or use of the substance or composition; through inadvertent error in these procedures; through differences in the manufacture, source, or purity of the ingredients employed to make or use the compositions or carry out the procedures; and the like.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The terms “non-naturally occurring” or “engineered” are used interchangeably and indicate the involvement of the hand of man. The terms, when referring to nucleic acid molecules or polypeptides mean that the nucleic acid molecule or the polypeptide is at least substantially free from at least one other component with which they are naturally associated in nature and as found in nature.

The term “gene” refers to a DNA sequence that comprises control and coding sequences necessary for the production of an RNA having a non-coding function (e.g., a ribosomal or transfer RNA), a polypeptide, or a precursor of any of the foregoing. The RNA or polypeptide can be encoded by a full-length coding sequence or by any portion of the coding sequence so long as the desired activity or function is retained. Thus, a “gene” refers to a DNA or RNA, or portion thereof, that encodes a polypeptide or an RNA chain that has functional role to play in an organism. For the purpose of this disclosure, it may be considered that genes include regions that regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, and locus control regions.

As used herein, “nucleic acid” or “nucleic acid sequence” refers to a polymer or oligomer of pyrimidine and/or purine bases, preferably cytosine, thymine, and uracil, and adenine and guanine, respectively (See Albert L. Lehninger, Principles of Biochemistry, at 793-800 (Worth Pub. 1982)). The present technology contemplates any deoxyribonucleotide, ribonucleotide, or peptide nucleic acid component, and any chemical variants thereof, such as methylated, hydroxymethylated, or glycosylated forms of these bases, and the like. The polymers or oligomers may be heterogenous or homogenous in composition and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. In some embodiments, a nucleic acid or nucleic acid sequence comprises other kinds of nucleic acid structures such as, for instance, a DNA/RNA helix, peptide nucleic acid (PNA), morpholino nucleic acid (see, e.g., Braasch and Corey, Biochemistry, 41 (14): 4503-4510 (2002)) and U.S. Pat. No. 5,034,506), locked nucleic acid (LNA; see Wahlestedt et al., Proc. Natl. Acad. Sci. U.S.A., 97:5633-5638 (2000)), cyclohexenyl nucleic acids (see Wang, J. Am. Chem. Soc., 122:8595-8602 (2000)), and/or a ribozyme. Hence, the term “nucleic acid” or “nucleic acid sequence” may also encompass a chain comprising non-natural nucleotides, modified nucleotides, and/or non-nucleotide building blocks that can exhibit the same function as natural nucleotides (e.g., “nucleotide analogs”); further, the term “nucleic acid sequence” as used herein refers to an oligonucleotide, nucleotide or polynucleotide, and fragments or portions thereof, and to DNA or RNA of genomic or synthetic origin, which may be single or double-stranded, and represent the sense or antisense strand. The terms “nucleic acid,” “polynucleotide,” “nucleotide sequence,” and “oligonucleotide” are used interchangeably. They refer to a polymeric form of nucleotides of any length, either deoxyribonucleotides or ribonucleotides, or analogs thereof.

A “peptide” or “polypeptide” is a linked sequence of two or more amino acids linked by peptide bonds. Peptides and polypeptides include proteins such as binding proteins, receptors, and antibodies. The terms “polypeptide” and “protein” are used interchangeably herein.

A “target cell” is a cell that is intended to receive or to eventually comprise one or more target DNA inserts of the present disclosure. In some embodiments, a target cell is a plant cell.

### 2. Gene Stacking System

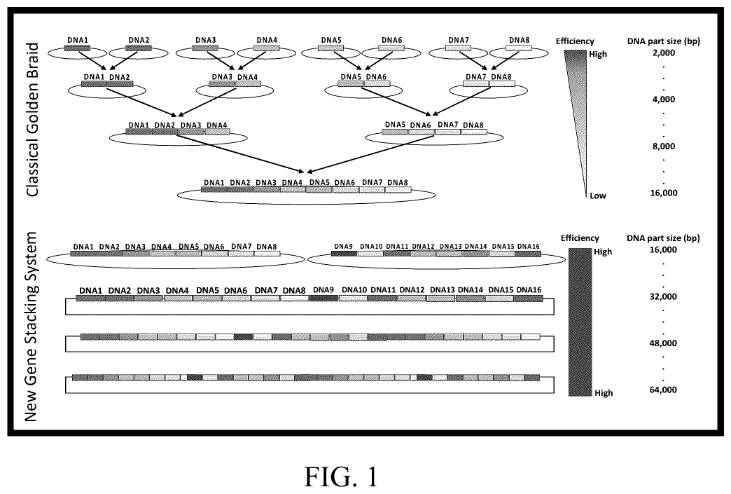

Standard vectors for implementation of synthetic biology methods in plants, including GoldenBraid (GB) technology vectors, have limited cargo DNA capacity of 25 kb or less, with cloning becoming inefficient when approaching the maximum size limit of the insert (FIG. 1). This is a major bottleneck in many synthetic biology applications that require the generation of large, multi-gene constructs. To address this problem, the present disclosure provides among other things, a GB-compatible gene stacking system that leverages the backbone of a bacterial artificial chromosome that has a cargo size limit of 300 kb, by far surpassing the 25 kb restriction imposed by GB.

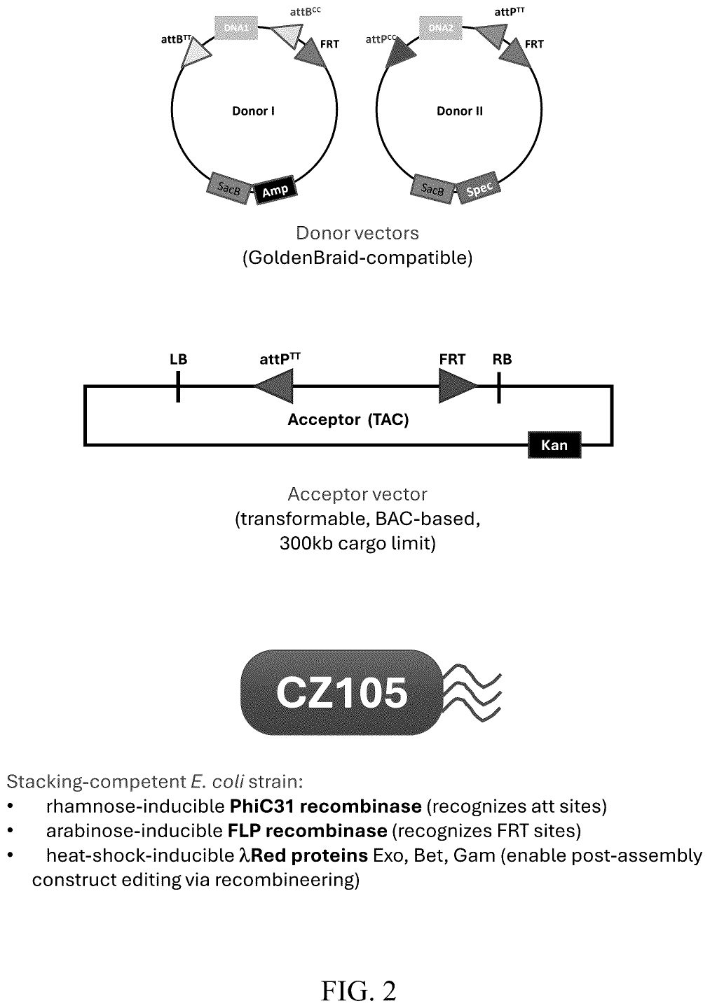

Disclosed herein are gene stacking systems. In some embodiments, a gene stacking system comprises donor vectors, an acceptor vector and an engineered bacterial cell comprising the acceptor vector. In some embodiments, a gene stacking system comprises two or more donor vectors, an acceptor vector and a gene-stacking competent bacterial strain. In some embodiments, a gene stacking system of the present disclosure comprises the components depicted in FIG. 2.

In some embodiments, a gene stacking system of the present disclosure comprises three types of components: GB-compatible donor vectors that deliver genes to be stacked, a BAC-based transformable acceptor vector with recombination sites compatible with that of donor vectors, and a gene stacking-competent strain of E. coli that carries three different types of inducible recombination machineries, PhiC31, FLP, and Lambda Red (FIG. 2). In some embodiments, donor vectors comprise att sites recognized by PhiC31 recombinase, an FRT site recognized by FLP recombinase, an antibiotic resistance gene (a selectable marker), and a SacB gene (for counterselection). In some embodiments, an acceptor vector comprises the att site, an FRT site and a selectable marker gene.

In some embodiments, a gene stacking system of the present disclosure combines GoldenBraid and recombineering technologies. In some embodiments, a BAC vector typically used in recombineering is modified to serve as an acceptor vector and four standard GoldenBraid vectors are modified to become donor vectors. A classical recombincering E. coli strain, SW105, harbors a temperature-inducible lambda Red recombineering system (that enables post-assembly construct modification via homologous recombination) and an arabinose-inducible FLP-FRT system (that can be leveraged for the removal of the donor vector backbone). To build the gene stacking system, either one or two additional recombinases (two options) need to be implemented to enable integration of donor vector DNA cargo into the acceptor vector. In some embodiments, the gene stacking system employs PhiC31, a well-studied serine integrase that mediates unidirectional recombination between attP and attB recognition sites. In some embodiments of the gene stacking system, two orthogonal attP and attB site pairs for PhiC31 are employed (instead of using two different recombinases recognizing different attPlattB sites).

In some embodiments, the present disclosure provides a gene stacking system comprising donor vectors, acceptor vectors, and an engineered E. coli. The classical E. coli SW105 strain expresses (A) lambda Red recombincering genes exo, beta, and gam in a heat-shock inducible manner enabling efficient homologous recombination and (B) FLP recombinase in an arabinose-inducible manner allowing for the removal of undesired parts of the construct (e.g., a selectable marker gene) after the recombination event. In some embodiments, the present disclosure provides extended capabilities of this bacterial strain by integrating a PhiC31 recombinase gene into the rhamnose catabolic operon, rhaBAD or the transporter gene rhaT in the genome of SW105, enabling rhamnose-inducible expression of PhiC31 in the resulting CZ105a and CZ105b strains. In some embodiments, PhiC31 and FLP recombinase sites and the SacB gene are introduced into the existing GB alpha- and omega-level destination vectors, converting them into donor vectors that can now donate their inserts to stacking-competent, acceptor vectors capable of harboring up to 300 kb of DNA cargo. In some embodiments, acceptor vectors utilize the backbone of transformable BAC (TAC) JATY vectors and carry PhiC31 and FLP recombinase sites compatible with that in the donor vectors. This enables integration of the entire donor plasmid sequence (backbone and insert) via PhiC31 into the TAC acceptor vector and easy subsequent removal of the donor vector backbone via FLP so that only the cargo of interest remains in the JATY clone. Follow-up rounds of PhiC31- and FLP-mediated recombinations enable the integration of additional genes from donor vectors to acceptor vectors to stack multiple inserts in an idempotent manner.

In some embodiments, the present disclosure provides an engineered E. coli configured to express PhiC31 in a tightly regulated manner. In some embodiments, PhiC31 with a partial ribosome binding site at the 5′ end is inserted into the E. coli SW105 genome by recombineering either immediately downstream of rhaA in the rhaBAD operon (strain CZ105a) or immediately downstream of rhaT to form an operon with rhaT (strain CZ105b). In some embodiments, the PhiC31 sequence comprises SEQ ID NO: 1. In some embodiments, the PhiC31 sequence comprises a partial Shine-Dalgarno (SD) sequence (ggagg) and spacer sequence (acaatc) at its 5′ end and after integration into the E. coli genome, the partial SD sequence and the stop codon (TAA) of the upstream gene form the optimal SD sequence. In some embodiments, the spacer sequence between the SD sequence and the start codon of PhiC31 comprises from 5 to 13 nucleotides.

In some embodiments, the present disclosure provides an acceptor vector engineered from a JATY transformable BAC pYLTAC (aka TAC) vector used as a backbone and its T-DNA region modified to make it suitable for recombinase-mediated DNA integration. In some embodiments, the acceptor vector comprises an attP^{TT} site to be used for the integration of a donor vector of interest. In some embodiments, the acceptor vector comprises an FRT site for the removal of the donor vector backbone.

In some embodiments, an acceptor vector is configured to contain up to about 300 kb of target DNA inserts. In some embodiments, an acceptor vector contains more than about 25 kb of target DNA inserts. In some embodiments, an acceptor vector contains less than or equal to about 300 kb of target DNA inserts.

In some embodiments, the rhamnose-mediated inducibility of PhiC31 and arabinose-mediated induction of FLP make the gene stacking system of the present disclosure tightly regulated and very efficient. Furthermore, the presence of the heat-inducible lambda Red machinery in the engineered E. coli strain allows for targeted post-assembly manipulation of acceptor vector constructs via recombincering. Without wishing to be bound to any one particular theory, this makes it possible to carry out single nucleotide editing, efficient insertion of small (up to 3 kb) DNA fragments, deletion of any part(s) of the construct of any size, and replacement of any-size fragment with up to 300 kb of other DNA at any stage of the acceptor construct assembly. Thus, a provided gene stacking system of the disclosure offers an exceptional level of post-cloning flexibility and is compatible with large cargo sizes of up to 300 kb, opening doors to the expression and post-cloning optimization of large, multi-gene pathways and complicated genetic circuits in plants.

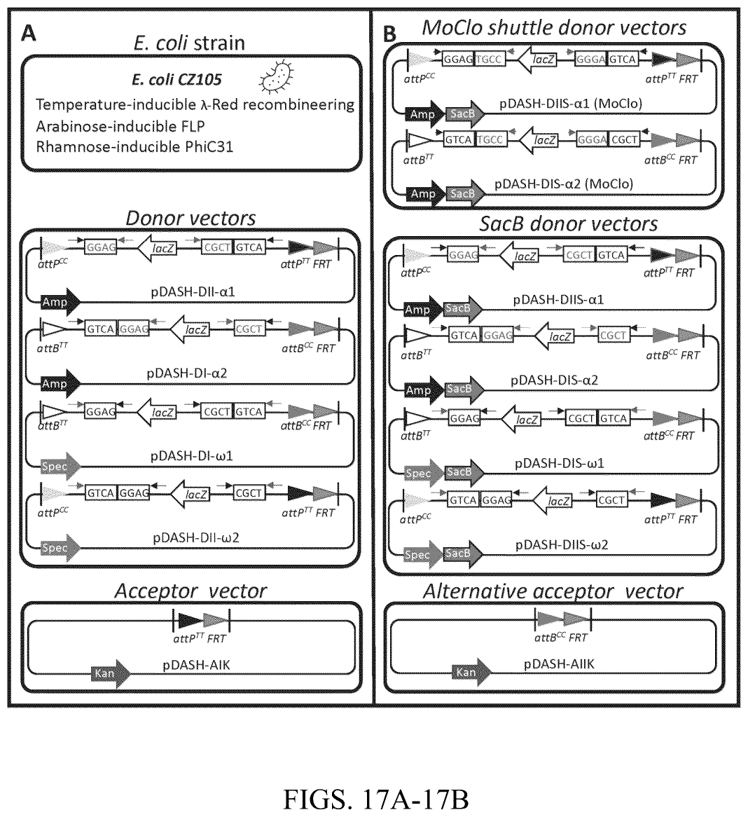

According to some embodiments, a gene stacking system of the present disclosure comprises the components depicted in FIGS. 16-17.

In some embodiments, a gene stacking system of the present disclosure is compatible with Agrobacterium-mediated transformation.

In some embodiments, a gene stacking system of the present disclosure is compatible not only with GB constructs, but also with Mobius Level-2 or Loop pEven plasmids and with Golden Gate multiplex gRNA vectors that all rely on the same standard grammar as GB and thus can donate their inserts to GB pDGB3 alpha-level donor vectors to then be moved to the stacking acceptor vector. In some embodiments, a gene stacking system of the present disclosure is compatible with most major Type IIS restriction enzyme-based cloning technologies currently utilized in plants.

In some embodiments, a gene stacking system of the present disclosure is compatible with all major Type IIS molecular cloning technologies. In some embodiments, a gene stacking system of the present disclosure comprises the components and constructs shown in Table 1. Donor GB vectors shown in Table 1 are compatible with Golden Gate, GoldenBraid, Mobius, and Loop. Donor MoClo vectors shown in Table 1 are compatible with MoClo.

TABLE 1
Illustrative gene stacking system constructs and compatibility
of the constructs with two Agrobacterium strains.

| E. coli | CZ105a | | | | CZ105b | | | |
|---|---|---|---|---|---|---|---|---|
| strain | | | | | | | | |
| Acceptor | pDASH-AIK | | | | pDASH-AIA | | | |
| vector | | | | | | | | |
| Donor | pDASH- | pDASH- | pDASH- | pDASH- | pDASH- | pDASH- | pDASH- | pDASH- |
| vector | DII-α1 | DI-α2 | DI-ω1 | DII-ω2 | DIIK-α1 | DIK-α2 | DIIK-α1 | DIK-α2 |
| | | | | | | | (MoClo) | (MoClo) |
| | Donor II | Donor I | Donor I | Donor II | Donor II | Donor I | Donor II | Donor I |
| Agro | GV3101 | | | | GV3101 ΔmltB3 | | | |
| strain | | | | | | | | |

In some embodiments, a gene stacking system comprises (a) at least one donor vector for delivering target DNA inserts; (b) a first acceptor vector configured to contain up to about 300 kb of target DNA inserts; and (c) a first engineered bacterial cell comprising the first acceptor vector. In some embodiments, the gene stacking system comprises two donor vectors, three donor vectors, four donor vectors, five donor vectors, six donor vectors, seven donor vectors, or eight donor vectors. In some embodiments, additional donor vectors may be part of the full gene stacking system, so that the full system comprises more than eight donor vectors. In some embodiments, the gene stacking system comprises two donor vectors. In some embodiments, the gene stacking system comprises four donor vectors. In some embodiments, the gene stacking system comprises eight donor vectors. In some embodiments, the gene stacking system comprises a second acceptor vector configured to contain up to about 300 kb of target DNA inserts and a second engineered bacterial cell comprising the second acceptor vector.

In some embodiments, an engineered bacterial cell comprises a recombineering system. In some embodiments, a first engineered bacterial cell of a gene stacking system comprises a recombineering system. In some embodiments, a second engineered bacterial cell of a gene stacking system comprises a recombineering system. In some embodiments, a first engineered bacterial cell and a second engineered bacterial cell of a gene stacking system each comprises a recombineering system. The recombineering system enables editing of the target DNA inserts and/or the acceptor vector. In some embodiments, the recombineering system comprises heat-shock-inducible lambda Red proteins.

In some embodiments, an engineered bacterial cell comprises an arabinose-inducible FLP recombinase. In some embodiments, a first engineered bacterial cell of a gene stacking system comprises an arabinose-inducible FLP recombinase. In some embodiments, a second engineered bacterial cell of a gene stacking system comprises an arabinose-inducible FLP recombinase. In some embodiments, a first engineered bacterial cell and a second engineered bacterial cell of a gene stacking system each comprises an arabinose-inducible FLP recombinase.

In some embodiments, an engineered bacterial cell comprises an inducible PhiC31 recombinase. For example, the inducible PhiC31 recombinase may be a rhamnose-inducible PhiC31 recombinase. In some embodiments, a first engineered bacterial cell of a gene stacking system comprises an inducible PhiC31 recombinase or a rhamnose-inducible PhiC31 recombinase. In some embodiments, a second engineered bacterial cell of a gene stacking system comprises an inducible PhiC37 recombinase or a rhamnose-inducible PhiC3/recombinase. In some embodiments, a first engineered bacterial cell and a second engineered bacterial cell of a gene stacking system each comprises an inducible PhiC3/recombinase or a rhamnose-inducible PhiC31 recombinase.

In some embodiments, an engineered bacterial cell is derived from an E. coli SW105 strain. In some embodiments, a first engineered bacterial cell of a gene stacking system is derived from an E. coli SW105 strain. In some embodiments, a second engineered bacterial cell of a gene stacking system is derived from an E. coli SW105 strain. In some embodiments, a first engineered bacterial cell and a second engineered bacterial cell of a gene stacking system each is derived from an E. coli SW105 strain. In some embodiments, the first engineered bacterial cell comprises a PhiC31 gene inserted immediately downstream of rhaA in the rhaBAD operon of the E. coli SW 105 genome. In some embodiments, the second engineered bacterial cell comprises a PhiC31 gene inserted immediately downstream of rhaT in the E. coli SW105 genome to form an operon with rhaT.

In some embodiments, the first acceptor vector comprises an attP^{TT} site, an FRT site, and a selection marker. In some embodiments, the second acceptor vector comprises an attP^{TT} site, an FRT site, and a selection marker. The selection marker may be an antibiotic resistance gene. In some embodiments, the first acceptor vector comprises a kanamycin resistance gene. In some embodiments, the second acceptor vector comprises an ampicillin resistance gene.

In some embodiments, each of the donor vectors of a gene stacking system comprises a first att site, a second att site, and an FRT site. In some embodiments, each of the donor vectors comprises a SacB gene. In some embodiments, each of the donor vectors comprises a selection marker. The selection marker may be an antibiotic resistance gene. For example, the antibiotic resistance gene may be selected from a kanamycin resistance gene, an ampicillin resistance gene, or a spectinomycin resistance gene.

In some embodiments, a gene stacking system of the disclosure is compatible with a GV3101 strain of Agrobacterium or a GV3101 derivate with a deletion in the mltB3 gene.

In some embodiments, each of the donor vectors of a gene stacking system is either a Donor I vector or a Donor II vector and the system is configured so that delivery of the target DNA inserts comprises introducing a Donor I vector or a Donor II vector into an engineered bacterial cell disclosed herein. In some embodiments, multiple target DNA inserts are introduced into the engineered bacterial cell and each target DNA insert is introduced from a Donor I vector or a Donor II vector in an alternating pattern. In select embodiments, the pattern comprises Donor I vector, Donor II vector, Donor I vector, Donor II vector, and so on.

In some embodiments, a gene stacking system of the disclosure is compatible with Type IIS restriction enzyme-based cloning technologies in plants.

In some embodiments, each of the donor vectors of a gene stacking system is configured to incorporate target DNA inserts from Golden Gate vectors, GoldenBraid plasmids, Mobius Level-2 plasmids, and Loop pEven plasmids. In some embodiments, each of the donor vectors of a gene stacking system is configured to incorporate target DNA inserts from MoClo plasmids. In some embodiments, each of the donor vectors of a gene stacking system is configured to incorporate target DNA inserts from either (i) Golden Gate vectors, GoldenBraid plasmids, Mobius Level-2 plasmids, and Loop pEven plasmids or (ii) MoClo plasmids.

In some embodiments, a gene stacking system of the present disclosure is compatible with Agrobacterium-mediated transformation.

In some embodiments, the present disclosure provides a gene stacking system comprising: (a) a first donor vector for delivering target DNA inserts; (b) a second donor vector for delivering target DNA inserts; (c) an acceptor vector configured to contain up to about 300 kb of target DNA inserts; and (d) an engineered bacterial cell comprising the acceptor vector, wherein the engineered bacterial cell is configured to express at least one recombinase. In some embodiments, the first donor vector is configured to deliver at least a first target DNA insert to the acceptor vector and the second donor vector is configured to deliver at least a second target DNA insert to the acceptor vector. In some embodiments, the first donor vector is configured to deliver multiple of the same or different target DNA inserts to the acceptor vector via one or more rounds of integration and the second donor vector is configured to deliver multiple of the same or different target DNA inserts to the acceptor vector via one or more rounds of integration. The target DNA inserts may all be different, all the same, or a combination thereof. In some embodiments, expression of the at least one recombinase by the engineered bacterial cell is inducible.

In some embodiments, an engineered bacterial cell comprises a recombineering system. The recombineering system enables editing of the target DNA inserts and/or the acceptor vector. For example, the editing may comprise: (i) single nucleotide editing; (ii) insertion of DNA fragments up to 300 kb; (iii) deletion of any parts of the acceptor vector; and/or (iv) replacement of any-size fragment of the acceptor vector with up to 300 kb of replacement DNA. In some embodiments, the recombineering system comprises heat-shock-inducible lambda Red proteins, said proteins comprising Exo, Beta and Gam.

In some embodiments of a gene stacking system disclosed herein, the first donor vector comprises attB recombination donor sites, the second donor vector comprises attP recombination donor sites, and the acceptor vector comprises an attP recombination acceptor site; or the first donor vector comprises attP recombination donor sites, the second donor vector comprises attB recombination donor sites, and the acceptor vector comprises an attB recombination acceptor site.

In some embodiments of a gene stacking system disclosed herein, expression of a first recombinase induces integration of the entire first donor vector into the acceptor vector; and/or expression of the first recombinase induces integration of the entire second donor vector into the acceptor vector. For example, if a first donor vector is introduced into the engineered bacterial cell, expression of a first recombinase induces integration of the entire first donor vector into the acceptor vector. Alternatively, if a second donor vector is introduced into the engineered bacterial cell, expression of the first recombinase induces integration of the entire second donor vector into the acceptor vector. In some embodiments, the first recombinase is expressed by the engineered bacterial cell in an inducible manner. In some embodiments, the first recombinase is a rhamnose-inducible PhiC3/recombinase.

In some embodiments of a gene stacking system disclosed herein, the first donor vector comprises a first recombination donor site that is compatible with a first recombination acceptor site, wherein said first recombination acceptor site is present in the acceptor vector prior to the integration of the entire first donor vector into the acceptor vector; and the second donor vector comprises a second recombination donor site that is compatible with a second recombination acceptor site, wherein said second recombination acceptor site is present in the acceptor vector prior to the integration of the entire second donor vector into the acceptor vector. In some embodiments, each of the recombination donor sites and each of the recombination acceptor sites are recognized by the same recombinase. In some embodiments, each of the recombination donor sites and each of the recombination acceptor sites are recognized by PhiC31 recombinase.

In some embodiments of a gene stacking system disclosed herein, the first donor vector comprises a first donor vector insert portion and a first donor vector backbone portion and expression of a second recombinase after the first donor vector has been integrated into the acceptor vector induces removal of the first donor vector backbone portion from the acceptor vector; and the second donor vector comprises a second donor vector insert portion and a second donor vector backbone portion and expression of a second recombinase after the second donor vector has been integrated into the acceptor vector induces removal of the second donor vector backbone portion from the acceptor vector. In some embodiments, the second recombinase is expressed by the engineered bacterial cell in an inducible manner. In some embodiments, the second recombinase is an arabinose-inducible FLP recombinase.

In some embodiments of a gene stacking system disclosed herein, the first donor vector comprises a first excision site; the second donor vector comprises a second excision site; and the acceptor vector comprises a third excision site. In some embodiments, the first, second, and third, excision sites are recognized by the same recombinase. In some embodiments, the first, second, and third, excision sites are FLP recombinase sites (also referred to herein as “FRT sites”). In some embodiments, the first donor vector and the second donor vector each comprise a SacB gene.

In some embodiments, the first donor vector, the second donor vector and the acceptor vector each comprise at least one selection marker. In some embodiments, the selection marker is an antibiotic resistance gene. In some embodiments, the first donor vector, the second donor vector and the acceptor vector each comprise a different selection marker.

In some embodiments, a gene stacking system of the disclosure is compatible with Type IIS restriction enzyme-based cloning technologies in plants. In some embodiments, the first donor vector and/or the second donor vector are configured to incorporate target DNA inserts from Golden Gate vectors, GoldenBraid plasmids, Mobius Level-2 plasmids, and Loop pEven plasmids. In some embodiments, the first donor vector and/or the second donor vector are configured to incorporate target DNA inserts from MoClo plasmids.

In some embodiments, a gene stacking system of the present disclosure is compatible with Agrobacterium-mediated transformation.

In some embodiments, the present disclosure provides a gene stacking system comprising: at least one donor vector comprising at least one target DNA insert flanked by recombination donor sites; at least one acceptor vector comprising recombination acceptor sites compatible with the recombination donor sites of the at least one donor vector, wherein the at least one acceptor vector is configured to contain up to about 300 kb of target DNA insert; and an engineered bacterial cell comprising the at least one acceptor vector, wherein the engineered bacteria cell is configured to express at least one recombinase. In some embodiments, expression of the at least one recombinase by the engineered bacterial cell is inducible. In some embodiments, the engineered bacterial cell further comprises a recombineering system. In some embodiments, the at least one donor vector is used for delivering multiple different target DNA inserts to the acceptor vector. In some embodiments, the gene stacking system is compatible with Type IIS restriction enzyme-based cloning technologies in plants. In some embodiments, the gene stacking system is compatible with Agrobacterium-mediated transformation.

In some embodiments, the present disclosure provides an engineered bacterial cell comprising: (a) a recombincering system; and (b) an acceptor vector configured to contain up to about 300 kb of target DNA inserts. In some embodiments, the bacterial cell is a lambda Red recombineering-competent E. coli. In some embodiments, the bacterial cell expresses: (i) exo, beta and gam genes in a heat-shock inducible manner; and (ii) a FLP recombinase in an arabinose-inducible manner. In some embodiments, the bacterial cell further expresses an inducible recombinase configured to mediate selective recombination between vectors in the bacterial cell. In some embodiments, the bacterial cell expresses an inducible PhiC3 recombinase. In some embodiments, the PhiC31 is expressed in a rhamnose-inducible manner. In some embodiments, the acceptor vector comprises sites recognized by PhiC31 recombinase and FLP recombinase.

In some embodiments, a gene stacking system of the present disclosure is compatible with Agrobacterium-mediated transformation.

### 3. Methods

The present disclosure also provides methods for gene stacking.

In some embodiments, a gene stacking method of the present disclosure comprises a series of steps that result in DNA fragments (e.g., target DNA inserts) stacked in an acceptor vector. In some embodiments, a first donor vector (Donor I vector) harboring DNA1 cargo is electroporated into CZ105 cells carrying the acceptor vector (Step 1). To induce integration of the Donor I vector into the acceptor vector, rhamnose is added to the growth medium (Step 2). Rhamnose induces PhiC31 expression that recognizes a compatible attB site in the Donor I vector and attP site in the acceptor vector. Recombination between these two sites is triggered and results in the integration of the entire Donor I vector into the acceptor vector. The attB and attP sites are converted into inactive attR and attL sites, respectively. After integration of the entire Donor I vector into the acceptor vector, the Donor I vector backbone is removed by activating FLP recombinase upon addition of arabinose to the media (Step 3). The trimmed acceptor vector carries the DNA1 of interest without the Donor I vector backbone. Then, to integrate DNA2 into the acceptor vector, steps 2 and 3 are repeated to first induce PhiC31 with rhamnose to integrate a Donor II vector into the acceptor vector and then to induce FLP with arabinose to delete the Donor II backbone from the acceptor vector. Steps 2 and 3 can be repeated to integrate DNA3, DNA4, etc. delivered by alternating Donor I vector and Donor II vectors. As before, the donor vectors are introduced into acceptor vector-harboring CZ105 via electroporation, PhiC31 recombination is triggered by the addition of rhamnose into the bacterial growth media to integrate a donor vector into the acceptor vector, and then FLP recombination is induced by arabinose addition to delete the donor backbone. Both recombination steps are carried out in vivo in the bacterial cells by manipulating the two sugars. After n rounds of PhiC31/FLP recombination, n DNA fragments will be stacked together in the acceptor vector.

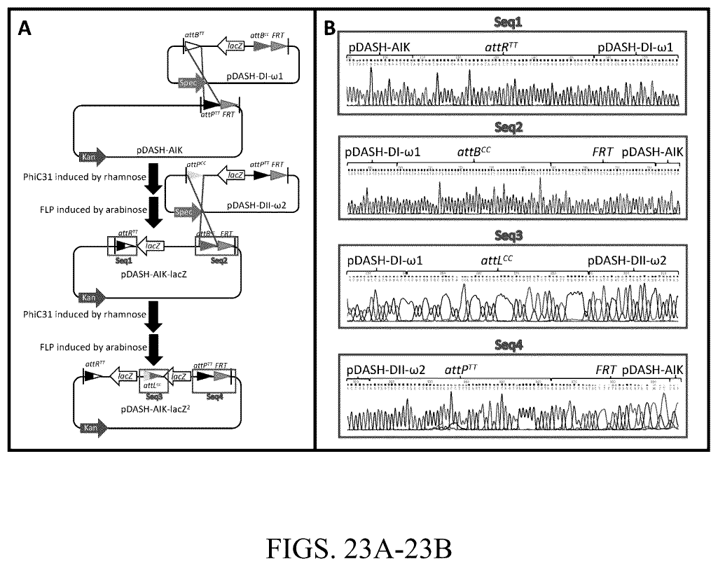

A gene stacking method according to an embodiment of the present disclosure is illustrated in FIGS. 23A-23B.

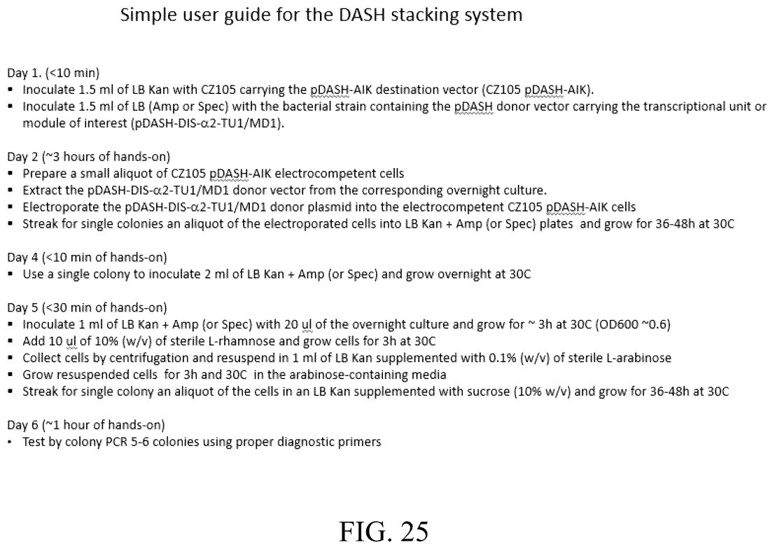

According to some embodiments of the present disclosure, an experimental timeline of gene stacking is illustrated in FIG. 25.

In some embodiments, the present disclosure provides a method of integrating target DNA inserts into an acceptor vector. In some embodiments, the present disclosure provides a method of integrating target DNA inserts into an acceptor vector, said method comprising a first round of integration comprising: (a) introducing a first donor vector into a gene stacking-competent bacterial cell carrying an acceptor vector, wherein the acceptor vector is configured to contain up to about 300 kb of target DNA inserts and wherein the first donor vector comprises a first donor vector backbone portion and a first donor vector insert portion; (b) inducing integration of the first donor vector into the acceptor vector; and (c) removing the first donor vector backbone portion from the acceptor vector; wherein, the first donor vector insert portion is configured to deliver target DNA inserts to the acceptor vector. In some embodiments, the method further comprises a second round of integration comprising: (d) introducing a second donor vector into the gene stacking-competent bacterial cell, wherein the second donor vector comprises a second donor vector backbone portion and a second donor vector insert portion; (c) inducing integration of the second donor vector into the acceptor vector; and (f) removing the second donor vector backbone portion from the acceptor vector, wherein the second donor vector insert portion is configured to deliver target DNA inserts to the acceptor vector. In some embodiments, the method further comprises additional rounds of integration, wherein each additional round of integration comprises: (g) introducing the first donor vector into the gene stacking-competent bacterial cell, h) inducing integration of the first donor vector into the acceptor vector; and i) removing the first donor vector backbone portion from the acceptor vector; or (j) introducing the second donor vector into the gene stacking-competent bacterial cell, k) inducing integration of the second donor vector into the acceptor vector; and l) removing the second donor vector backbone portion from the acceptor vector; wherein the method comprises alternating between parts (g)-(i) and parts (j)-(l); and wherein each additional round of integration delivers target DNA inserts to the acceptor vector. In some embodiments, introducing a donor vector into the gene stacking-competent bacterial cell comprises a molecular biology method of introducing nucleic acid molecules such as plasmids or vectors into cells. In some embodiments, introducing a donor vector into the gene stacking-competent bacterial cell comprises electroporation. In some embodiments, the first donor vector is configured to deliver multiple of the same or different target DNA inserts to the acceptor vector via one or more rounds of integration. In some embodiments, the second donor vector is configured to deliver multiple of the same or different target DNA inserts to the acceptor vector via one or more rounds of integration. In some embodiments, inducing integration of the first donor vector comprises induction of recombination between the first donor vector and the acceptor vector. In some embodiments, inducing integration of the second donor vector comprises induction of recombination between the second donor vector and the acceptor vector. In some embodiments, removing the first donor vector backbone portion from the acceptor vector comprises excision of the first donor backbone portion from the acceptor vector via homologous recombination mediated by FLP recombinase. In some embodiments, removing the second donor vector backbone portion from the acceptor vector comprises excision of the second donor backbone portion from the acceptor vector via homologous recombination mediated by FLP recombinase. In some embodiments, the method further comprises editing of the integrated DNA inserts and/or acceptor vector. In some embodiments, the editing comprises: (i) single nucleotide editing; (ii) insertion of DNA fragments up to 300 kb; (iii) deletion of any parts of the acceptor vector; and/or (iv) replacement of any-size fragment of the acceptor vector with up to 300 kb of replacement DNA. In some embodiments, the editing comprises using a recombineering system. In some embodiments, the gene stacking-competent bacterial cell comprises a recombineering system. In some embodiments, the recombineering system comprises heat-shock-inducible lambda Red proteins, said proteins comprising Exo, Beta and Gam. In some embodiments, the method further comprises delivering a part of the acceptor vector comprising the target DNA inserts to a target cell. For example, the target cell is a yeast, bacteria, or plant cell. In some embodiments, the target cell is a plant cell. In some embodiments, delivering a part of the acceptor vector to the target cell comprises Agrobacterium-mediated transformation.

The gene stacking systems of the present disclosure provide a variety of advantages. In some embodiments, an advantage of the provided gene stacking systems is that everything takes place in vivo, so there is no need to buy expensive recombinases. Rhamnose and arabinose are relatively inexpensive. In some embodiments, an advantage of the provided gene stacking systems is a relatively large cargo size limit. Since a BAC backbone is utilized in the construction of the acceptor vector, large gene stacks of up to 300 kb should be tolerated, unlike standard GoldenBraid vectors that have a cargo size limit of about 25 kb. In some embodiments, an advantage of the provided gene stacking systems is the ability to edit assembled constructs. The assemblies in the acceptor vector are not final and can be edited using the lambda Red recombination machinery inducible by heat treatment (e.g., 15 min at 42° C.). For example, without wishing to be bound to any one particular theory, if a researcher wants to swap a promoter in one of the genes, to replace or remove a plant selectable marker gene, or to introduce a specific missense mutation to alter the activity of one of the enzymes encoded by the cassette, this can be easily accomplished via recombineering. In some embodiments, an advantage of the provided gene stacking systems is the system's compatibility with all major Type IIS molecular cloning technologies, including Golden Gate, GoldenBraid, Mobius, MoClo and Loop. Accordingly, without wishing to be bound to any one particular theory, DNA parts made in one vector type (e.g., Mobius vectors) can be transferred to the gene stacking system donor vectors via a simple Type IIS enzyme-based subcloning step.

In some embodiments, the full system comprises: two versions of CZ105 strains of E. coli, two acceptor vectors (one with Kan and another with Amp resistance), and 8 donor vectors. Amp- and Spec-resistant vectors are compatible with the standard GV3101 strain of Agro, whereas Kan-resistant vectors require the use of a GV3101 derivate with a deletion in the mltB3 gene.

### 4. Kits

Embodiments of the present disclosure also provide kits comprising a gene stacking system or one or more components thereof, as disclosed herein.

The kits can also comprise other agents and/or products co-packaged, co-formulated, and/or co-delivered with other components. The kits can also comprise instructions for using the components of the kit. The instructions are relevant materials or methodologies pertaining to the kit. The materials may include any combination of the following: background information, list of components, brief or detailed protocols for using the compositions, trouble-shooting, references, technical support, and any other related documents. Instructions can be supplied with the kit or as a separate member component, either as a paper form or an electronic form which may be supplied on computer readable memory device or downloaded from an internet website, or as recorded presentation.

It is understood that the disclosed kits can be employed in connection with the disclosed methods. The kit may further contain containers or devices for use with the methods or compositions disclosed herein.

The kits provided herein are in suitable packaging. Suitable packaging includes, but is not limited to, vials, bottles, jars, flexible packaging, and the like. Individual member components of the kits may be physically packaged together or separately.

### 5. Materials and Methods

### Generation of PhiC31 Integrase Attachment Site (Att) and Flippase Recombinase Target (FRT)

All att sites, including attP^{TT},

(SEQ ID NO: 4)
AGTAGTGCCCCAACTGGGGTAACCTTTGAGTTCTCTCAGTTGGGGGCGT
A,
attB^{TT}
(SEQ ID NO: 5)
CCGCGGTGCGGGTGCCAGGGCGTGCCCTTGGGCTCCCCGGGCGCGTACT
CC,
and
their orthogonal attP^{CC}
(SEQ ID NO: 6)
(AGTAGTGCCCCAACTGGGGTAACCTCCGAGTTCTCTCAGTTGGGGGCG
TA)
and
attB^{CC}
(SEQ ID NO: 7)
(CCGCGGTGCGGGTGCCAGGGCGTGCCCCCGGGCTCCCCGGGCGCGTAC
TCC) sites,
FRT,
(SEQ ID NO: 8)
GAAGTTCCTATACTTTCTAGAGAATAGGAACTTC,

and combined att-FRT sites, were generated by PCR using primers listed in Table 2 and iProof High-Fidelity DNA Polymerase (bio-rad.com).

Table 2: Incorporation of the PhiC31 integrase attachment site (att) and flippase recombinase target site (FRT) in DASH system vectors.

| | PCR | | | |
|---|---|---|---|---|
| Goal | template | Method | Primer sequence | Product |
| Generation of the | | | | |
| attP and FRT sites | | | | |
| for the acceptor | | | | |
| vector | | | | |
| pDASH-AIK | pYLTAC17 | PCR | P27, | Left flanking |
| | | | TTAATAACACATTGCGGACG | sequence (F1) |
| | | | T (SEQ ID NO: 9) | |
| | | | P83, | |
| | | | GGGCACTACTAGAATTCAGT | |
| | | | ACATTAAAAACGTC (SEQ ID | |
| | | | NO: 10) | |
| | overlapping | PCR | P84, | attP^{TT}-FRT (F2) |
| | primers | | ACTGAATTCTAGTAGTGCCCC | |
| | | | AACTGGGGTAACCTTTGAGTT | |
| | | | CTC (SEQ ID NO: 11) | |
| | | | P85, | |
| | | | ctctagaaagtataggaacttcTACGCC | |
| | | | CCCAACTGAGAGAACTCAAAG | |
| | | | GTTACCCC (SEQ ID NO: 12) | |
| | PYLTAC17 | PCR | P86, | Right flanking |
| | | | gaagttcctatactttctagagaatagg | sequence (F3) |
| | | | aacttcGAATTCGTAGGGATAACAGG | |
| | | | (SEQ ID NO: 13) | |
| | | | P28, | |
| | | | CGGAGAATTAAGGGAATTAC | |
| | | | C (SEQ ID NO: 14) | |
| | | Gibson | | pCR2.1_attP^{TT}-FRT |
| | | assembly | | (acceptor) |
| | | F1, F2, F3 | | |
| Generation of the | | | | |
| attP and FRT sites | | | | |
| for the alpha 1 | | | | |
| donor vector | | | | |
| pDASH-DII-α1 | overlapping | PCR | P71, | pCR2.1_attP^{CC} |
| | primers | | taaacataacgaattcAGTAGTGCCCC | (alpha1) |
| | | | AACTGGGGTAACCTCCGAGT | |
| | | | TCTCTCA (SEQ ID NO: 15) | |
| | | | P72, | |
| | | | tgtgggtctctctcctgagacgTACGCC | |
| | | | CCCAACTGAGAGAACTCGGAG | |
| | | | GTTAC (SEQ ID NO: 16) | |
| | pCR2.1_ | PCR | P73, | pCR2.1_attP^{TT}_FRT |
| | attP^{TT}-FRT | | ggtctctcgctgtcatgagacgAGTAGT | (alpha1) |
| | (acceptor) | | GCCCCAACTGGGGT (SEQ ID | |
| | | | NO: 17) | |
| | | | P74, | |
| | | | tatcctgtcagaattcgaagttcctatt | |
| | | | ctctagaaag (SEQ ID NO: 18) | |
| Generation of the | | | | |
| attP and FRT sites | | | | |
| for the alpha 2 | | | | |
| donor vector | | | | |
| pDASH-DI-α2 | pCR2.1_ | PCR | P75, | pCR2.1_attB^{TT} |
| | attB^{TT} | | taaacataacaagcttCCGCGGTGCGG | (alpha2) |
| | (omega1) | | GTGCCAGGG (SEQ ID NO: 19) | |
| | | | P76, | |
| | | | ggtctctctcctgactgagacgGGAGTA | |
| | | | CGCGCCCGGGGAGC (SEQ ID | |
| | | | NO: 20) | |
| | pCR2.1_ | PCR | P77, | pCR2.1_attB^{CC}-FRT |
| | attB^{CC}-FRT | | ctggggtctctcgcttgagacgCCGCGG | (alpha2) |
| | (omega1) | | TGCGGGTGCCAGGG (SEQ ID | |
| | | | NO: 21) | |
| | | | P78, | |
| | | | tatcctgtcaaagcttgaagttcctatt | |
| | | | ctctagaaag (SEQ ID NO: 22) | |
| Generation of the | | | | |
| attP and FRT sites | | | | |
| for the omega 1 | | | | |
| donor vector | | | | |
| pDASH-DI-ω1 | overlapping | PCR | P67, | pCR2.1_attB^{TT} |
| | primers | | taaacataacggatccCCGCGGTGCG | (omega1) |
| | | | GGTGCCAGGGCGTGCCCTTG | |
| | | | GGCTCC (SEQ ID NO: 23) | |
| | | | P68, | |
| | | | tgtgcgtctcactcctgagaccGGAGTA | |
| | | | CGCGCCCGGGGAGCCCAAGGG | |
| | | | CACGCCCT (SEQ ID NO: 24) | |
| | overlapping | PCR | P69, | attB^{CC} (F4) |
| | primers | | cgtctcacgctgtcatgagaccCCGCGG | |
| | | | TGCGGGTGCCAGGGCGTGCCC | |
| | | | CCGGGCTCC (SEQ ID NO: 25) | |
| | | | P87, | |
| | | | taggaacttcGGAGTACGCGCCCG | |
| | | | GGGAGCCCGGGGGCACGCCC | |
| | | | TG (SEQ ID NO: 26) | |
| | pCR2.1_ | PCR | P88, | FRT (F5) |
| | attP^{TT}-FRT | | CGCGTACTCCgaagttcctatactttct | |
| | (acceptor) | | agag (SEQ ID NO: 27) | |
| | | | P70, | |
| | | | tatcctgtcaggatccgaagttcctatt | |
| | | | ctctagaaag (SEQ ID NO: 28) | |
| | attB^{CC} (F4), | overlapping | P69, | pCR2.1_attB^{CC}-FRT |
| | FRT (F5) | PCR | cgtctcacgctgtcatgagaccCCGCGG | (omega1) |
| | | | TGCGGGTGCCAGGGCGTGCCC | |
| | | | CCGGGCTCC (SEQ ID NO: 29) | |
| | | | P70, | |
| | | | tatcctgtcaggatccgaagttcctatt | |
| | | | ctctagaaag (SEQ ID NO: 30) | |
| Generation of the | | | | |
| attP and FRT sites | | | | |
| for the omega 2 | | | | |
| donor vector | | | | |
| pDASH-DII-ω2 | pCR2.1_ | PCR | P79, | pCR2.1_attP^{CC} |
| | attp^{CC} | | tgtaaacataacgatatcAGTAGTGCCC | (omega2) |
| | (alpha1) | | CAACTGGGGT (SEQ ID NO: | |
| | | | 31) | |
| | | | P80, | |
| | | | ctgactgagaccgatTACGCCCCCAA | |
| | | | CTGAGAGAA (SEQ ID NO: 32) | |
| | pCR2.1_ | PCR | P81, | pCR2.1_attP^{TT}-FRT |
| | attP^{TT}-FRT | | acgcttgagaccgatAGTAGTGCCCC | (omega2) |
| | (acceptor) | | AACTGGGGT (SEQ ID NO: 33) | |
| | | | P82, | |
| | | | tatatcctgtcagatatcgaagttccta | |
| | | | ttctctagaaag (SEQ ID NO: 34) | |

Briefly, to generate the attP^{TT}-FRT site flanked by 40 bp of homology to each side of the desired insertion site in the pYLTAC17 vector, these flanking sequences were first amplified using the primers P27/P83 and P86/P28 (see Table 2) and the pYLTAC17 vector as a template, and the attP^{TT}-FRT site sequence was obtained by PCR with the overlapping primers P84/P85 (Table 2). The resulting three fragments were assembled together by Gibson assembly (neb.com) and cloned into pCR2.1 (thermofisher.com) according to the user manuals, generating pCR2.1_attP^{TT}-FRT (acceptor).

Similarly, to obtain the attB^{CC}-FRT sequence, the FRT sequence was amplified from pCR2.1_attP^{TT}-FRT (acceptor) described above using primers P88/P70, while attB^{CC} was generated with overlapping primers P69/P87. The resulting two fragments were assembled together via overlapping PCR with primers P69/P70 and cloned into pCR2.1, yielding pCR2.1_attB^{CC}-FRT (omega1).

For the other seven att or att-FRT sites used to generate the different DASH donor vectors, a similar strategy was employed, utilizing either overlapping primers or amplifying the corresponding att-FRT plasmids as indicated in Table 2.

All PCR products were purified using QIAquick Gel Extraction Kit (qiagen.com) and cloned into pCR2.1 (thermofisher.com) according to the user manuals. All att and/or FRT sites in pCR2.1 were confirmed by Sanger sequencing.

### Donor Vector Modification

Replacement of the kanamycin for the ampicillin resistance gene. The kanamycin gene of the GoldenBraid vectors, pDGB3α1 and pDGB3α2, was replaced with a domesticated ampicillin gene [(to remove an internal BsaI site, according to the GoldenBraid cloning (goldenbraidpro.com)] by recombineering. Briefly, the domesticated ampicillin gene sequence was amplified using primers Amp rep Kan alpha f and r and the PCR product was used to replace the kanamycin sequences used standard recombineering procedures. The resulting ampicillin version of pDGB3α1 and pDGB3α2, along with the original GoldenBraid vectors, pDGB3ω1 and pDGB3ω2, were utilized for subsequent donor vector modification.

Insertion of the att and att-FRT sites in the donor vectors. To insert att and att-FRT just outside of the recognition sequence of a Type IIS restriction enzyme, BsaI or BsmBI, in the four donor vectors, the vectors were first linearized with restriction enzymes listed in Table 3.

TABLE 3
Incorporation of the att and att-FRT sites in donor vectors.

| Fragment 1 | Fragment 2 | Fragment 3 | Fragment 4 |
|---|---|---|---|
| (backbone) | (LacZ) | (att) | (att-FRT) |
| digestion by | Amplified from | attP^{CC} was amplified from | attP^{TT}-FRT was amplified from |
| EcoR I | pDGB3α1 by PCR with | pCR2.1_attP^{CC} (alpha1) by PCR | pCR2.1_attP^{TT}-FRT (alpha1) by |
| | primers Alpha1 f/r | with primers P71-2/P72 | PCR with primers P73/P74-2 |
| digestion by | Amplified from | attB^{TT} was amplified from | attB^{CC}-FRT was amplified from |
| HindIII | pDGB3α2 by PCR with | pCR2.1_attB^{TT} (alpha2) by PCR | pCR2.1_attB^{CC}-FRT (alpha2) |
| | primers Alpha2 f/r | with primers P75-2/P76 | by PCR with primers P77/P78-2 |
| digestion by | digestion by | attB^{TT} was amplified from | attB^{CC}-FRT was amplified from |
| BamHI | BsaI | pCR2.1_attB^{TT} (omega1) by PCR | pCR2.1_attB^{CC}-FRT (omega1) |
| | | with primers P67/P68 | by PCR with primers P69/P70 |
| digestion by | digestion by | attP^{CC} was amplifie from | attP^{TT}-FRT was amplified from |
| EcoRV | EcoRV | pCR2.1_attP^{CC} (omega2) by PCR | pCR2.1_attP^{TT}-FRT (omega2) |
| | | with primers P79/P80 | by PCR with primers P81/P82 |

The backbones of all vectors and the LacZ cassettes for pDGB301 and pDGB3ω2 were gel-purified. For pDGB3α1 and pDGB3α2 (ampicillin versions), since the LacZ cassettes contain restriction recognition sites for the restriction enzymes used to linearize the vectors, the intact LacZ cassettes were re-amplified from pDGB3α1 and pDGB3α2 by PCR with primers listed in Table 4.

Table 4: Primers used for the generation of the DASH donor vectors and the CZ105 E. coli strains.

| 1) Donor vector | | |
|---|---|---|
| modifications | | |
| Use | Primer name | Primer sequence |
| Amplification of att or | P71-2 | atattgtggtgtaaacataacgaattcAGTAGTG (SEQ ID NO: |
| att-FRT in Table S2 | | 35) |
| | P74-2 | accegccaatatatcctgtcagaattcgaag (SEQ ID NO: 36) |
| | P75-2 | atattgtggtgtaaacataacaagcttCCGC (SBQ ID NO: 37) |
| | P78-2 | acccgccaatatatcctgtcaaagcttgaagt (SEQ ID NO: 38) |
| Amplification of the LacZ | Alpha1 f | cgtctcaggagagagaccca (SEQ ID NO: 39) |
| cassette in Table S2 | Alpha1 r | cgtctcatgacagcgagaga (SEQ ID NO: 40) |
| | Alpha2 f | cgtctcagtcaggagagaga (SEQ ID NO: 41) |
| | Alpha2 f | cgtctcaagcgagagacccca (SEQ ID NO: 42) |
| Donor vector construction | MoClo alpha1 f | CGTAcgtctcaggagTGCCagagacccacagcttgtctg (SEQ |
| (MoClo version) | | ID NO: 43) |
| | MoClo alpha1 r | TACTcgtctcatgacTCCCagagaccccagctggcacga (SEQ |
| | | ID NO: 44) |
| | MoClo alpha2 f | CTCCcgtctcagtcaTGCCagagacccacagcttgtctg (SEQ |
| | | ID NO: 45) |
| | MoClo alpha2 r | GCGGcgtctcaagcgTCCCagagaccccagctggcacga |
| | | (SEQ ID NO: 46) |
| Ampicillin resistance gene | Amp 1 f2 | GCGCCGTCTCGCTCGAATGAGTATTCAACAT |
| domestication | | TTCCGT (SEQ ID NO: 47) |
| | Amp 1 r | GCGCCGTCTCGGGGACCCACGCTCACCGGCT |
| | | (SEQ ID NO: 48) |
| | Amp 2 f | GCGCCGTCTCGTCCCGCGGTATCATTGCAGC |
| | | (SEQ ID NO: 49) |
| | Amp 2 r | GCGCCGTCTCGCTCAAAGCTTACCAATGCTT |
| | | AATCAGTGAGG (SEQ ID NO: 50) |
| Replacement of the | Amp rep Kan alpha | aaatactgtagaaaagaggaaggaaataataagttctgttATGAGT |
| Kanamycin by the | f | ATTCAACATTTCCG (SEQ ID NO: 51) |
| Ampicillin resistance gene | Amp rep Kan alpha | aaactcacgttaagggattttggtcatgcattctaggtgaTTACCAA |
| in alpha vectors | r | TGCTTAATCAGTG (SEQ ID NO: 52) |
| SacB domestication | P47 | GCGCCGTCTCGCTCGTCGAGCTTGACATTGT |
| | | AGGA (SEQ ID NO: 53) |
| | P48 | GCGCCGTCTCGAAACGCCGTACGTTTCTTTG |
| | | T (SEQ ID NO: 54) |
| | P49 | GCGCCGTCTCGGTTTCTCATATTACACGCCAT |
| | | G (SEQ ID NO: 55) |
| | P50 | GCGCCGTCTCGCATCTCCGTCAAAAATCGTT |
| | | TTG (SEQ ID NO: 56) |
| | P51 | GCGCCGTCTCGGATGGAAAAACATATCAGA |
| | | ACGTT (SEQ ID NO: 57) |
| | P52 | GCGCCGTCTCGGGATCTCTCAGCGTATGGTT |
| | | (SEQ ID NO: 58) |
| | P53 | GCGCCGTCTCGATCCTCACTACGTTGAAGAC |
| | | A (SEQ ID NO: 59) |
| | P54 | GCGCCGTCTCGCTCAAAGCGGTATAGGAACT |
| | | TCCCTTAG (SEQ ID NO: 60) |
| Replacement of the | Kan-SacB f | aaatactgtagaaaagaggaaggaaataataagttctgttatgagcca |
| Ampicillin or Spectin- | | tattcaacggga (SEQ ID NO: 61) |
| omycins equences by the | Kan r | GTTCATGATTGTCCTCCttagaaaaactcatcgagcatca |
| Kan-SacB casette in the | | (SEQ ID NO: 62) |
| alpha and omega vectors, | SacB f | taaGGAGGACAATCATGAACATGAACATCAAA |
| respectively | | AAAATTGTAAAAC (SEQ ID NO: 63) |
| | Kan-SacB r | aaactcacgttaagggattttggtcatgcattctaggtgaTTAGTTG |
| | | ACTGTCAGCTGTC (SEQ ID NO: 64) |
| Replacement of the | Amp rep Kan alpha | aaatactgtagaaaagaggaaggaaataataagttctgttATGAGT |
| Kanamycin sequence in the | f | ATTCAACATTTCCG (SEQ ID NO: 65) |
| Kan-SacB casette by | Amp rep Kan r2 | ACAATTTTTTTGATGTTCATGTTCATGATTGT |
| Ampicillin in alpha | | CCTCCttaCCAATGCTTAATCAGTGAGG (SEQ |
| vectors with SacB | | ID NO: 66) |
| Replacement of the | Spec rep Kan f | aaatactgtagaaaagaggaaggaaataataagttctgttccagccag |
| Kanamycin sequence in the | | gacagaaatgc (SEQ ID NO: 67) |
| Kan-SacB casette by | Spec rep Kan r | ACAATTTTTTTGATGTTCATGTTCATGATTGT |
| Spectinomycin in the omega | | CCTCCttatttgccgactaccttggtga (SEQ ID NO: 68) |
| vectors with SacB | | |
| Delection of the extra | P65 | aacagaacttattatttccttcctc (SEQ ID NO: 69) |
| Spectinomycin promoter | alpha 2800 r | gatctattcagatagcagctc (SEQ ID NO: 70) |
| sequence in Omega vectors | alpha 2800 f | gagctgctatctgaatagatc (SEQ ID NO: 71) |
| | P66 | tcacctagaatgcatgacca (SEQ ID NO: 72) |
| | Spec rep Amp f | aaatactgtagaaaagaggaaggaaataataagttctgttatgcgctc |
| | | acgcaactggt (SEQ ID NO: 73) |
| | Kan-SacB r | aaactcacgttaagggattttggtcatgcattctaggtgaTTAGTTG |
| | | ACTGTCAGCTGTC (SEQ ID NO: 74) |
| 2) Generation of two | | |
| CZ105 strains of E. coli | | |
| Use | Primer name | Primer sequence |
| Insertion of PhiC31 | rhaA-Amp f | gcgggcttatgagaaagaaattttgagtcgccgcgggtaaggaggaca |
| downstream of rhaA to | | atcGGAGGTGGAGGTGGAGCT (SEQ ID NO: 75) |
| make CZ105a | rhaA-Amp r | cgtaggccggataaggcgctcgcgccgcatccggcagtgtGGCCC |
| | | CAGCGGCCGCAGCAG (SEQ ID NO: 76) |
| | rhaA-phiC31 f | agaaagaaattttgagtcgccgcgggtaaggaggacaatcATGGA |
| | | CACGTACGCGGGTGC (SEQ ID NO: 77) |
| | rhaA-phiC31 r | cgtaggccggataaggcgctcgcgccgcatccggcagtgtCTACG |
| | | CCGCTACGTCTTCCG (SEQ ID NO: 78) |
| Insertion of PhiC31 | rhaTF | TGGTGATTATTGTCGCCGCTAACATCGTCGG |
| downstream of rhaT to | | CATCGGCATGGCGAATTAAggaggacaatcGGAG |
| make CZ105b | | GTGGAGGTGGAGCT (SEQ ID NO: 79) |
| | rhaTR | ACCGGGATGACGCCCAGCCAGTGGCGTCATC |
| | | TCAATTCGCAGAAAGATTAGGCCCCAGCGGC |
| | | CGCAGCAGCACC (SEQ ID NO: 80) |
| | phiC31rhaF | GTCGCCGCTAACATCGTCGGCATCGGCATGG |
| | | CGAATTAAggaggacaatcATGGACACGTACGCG |
| | | GGTGCTTACG (SEQ ID NO: 81) |
| | phiC31 rhaR3 | ACCGGGATGACGCCCAGCCAGTGGCGTCATC |
| | | TCAATTCGCAGAAAGATTAaCTACGCCGCTA |
| | | CGTCTTCCG (SEQ ID NO: 82) |

In addition, all att and att-FRT sites for each vector were also amplified from their corresponding pCR2.1-based plasmids listed in Table 2 by PCR with primers shown in Tables 2 and 4. Four DNA fragments (backbone, LacZ cassette, att, and att-FRT) for each donor vector were assembled by Gibson assembly (neb.com), generating pDASH-DII-α1, pDASH-DI-α2, pDASH-DI-ω1 and pDASH-DII-ω2.

SacB domestication and insertion into donor vectors. The SacB gene was domesticated to remove the one BsaI and two BsmBI recognition sites with primers (P47-P54) listed in Table 4 from JMA1300 and cloned into pUPD2, according to GoldenBraid cloning. Partial Shine-Dalgarno sequence (GGAGG) and spacer sequence (ACAATC) followed by the domesticated SacB was inserted just downstream of the stop codon of the ampicillin gene in pDASH-DII-α1 and pDASH-DI-α2 or the spectinomycin gene in pDASH-DI-ω1 and pDASH-DII-ω2, forming an operon with the upstream antibiotic gene, by recombineering, which was performed in two steps. First, the ampicillin gene in donor alpha vectors or the spectinomycin gene in donor omega vectors was replaced with a kanamycin-SacB PCR fragment amplified with the primers listed in Table 4 by recombineering. In the second step, the kanamycin gene in all vectors was replaced with ampicillin in alpha or spectinomycin in omega vectors. For that, the ampicillin gene was first amplified using pDASH-DII-α1 DNA as a template and the primers Amp rep Kan alpha f and Amp rep Kan r2. The resulting PCR product was used to replace the kanamycin gene in the alpha vectors used standard recombineering procedures, generating pDASH-DIIS-α1 and pDASH-DIS-α2.

Similarly, the primers Spec rep Kan f and r were used to amplify by PCR the spectinomycin sequences using DNA from the pDASH-DIS-ω1 vector and used in a recombincering experiment to replace the kanamycin sequences in the omega vectors. Due to the toxicity of SacB in the pDASH omega vectors, extra spectinomycin promoter sequences were removed by Gibson assembly (neb.com) using the fragments amplified by PCR with primers listed in Table 4, generating pDASH-DIS-ω1 or pDASH-DIIS-ω2.

Generation of MoClo-compatible alpha donor vectors. The LacZ cassettes were amplified by PCR from GoldenBraid pDGB3α1 and pDGB3α2 with primers listed in Table 4, which introduced the MoClo-compatible assembly syntax. The resulting PCR products were purified, digested with BsmBI, and ligated with DASH donor vectors pDASH-DIIS-α1 and pDASH-DIS-α2 pre-digested with the same enzyme.

### Acceptor Vector Modification

The fragment containing attP^{TT}-FRT with homology arms of 40 bp was amplified by PCR from the plasmid pCR2.1_attP^{TT}-FRT (acceptor) with primers P27 and P28 (Table 2). The purified PCR products were used to replace the SacB cassette and the RPSL-Amp operon in the T-DNA region of JMA2450 by recombineering, generating pDASH-AIK. For the second acceptor vector, pDASH-AIIK, plasmid pDASH-AIK-LacZ was digested by BsaI, filled in by Klenow (neb.com), and re-ligated by T4 DNA ligase. Both acceptor vectors were confirmed by whole plasmid sequencing and/or Sanger sequencing.

E. coli SW105 Strain Modification for PhiC31 Expression Under a Rhamnose-Inducible Promoter

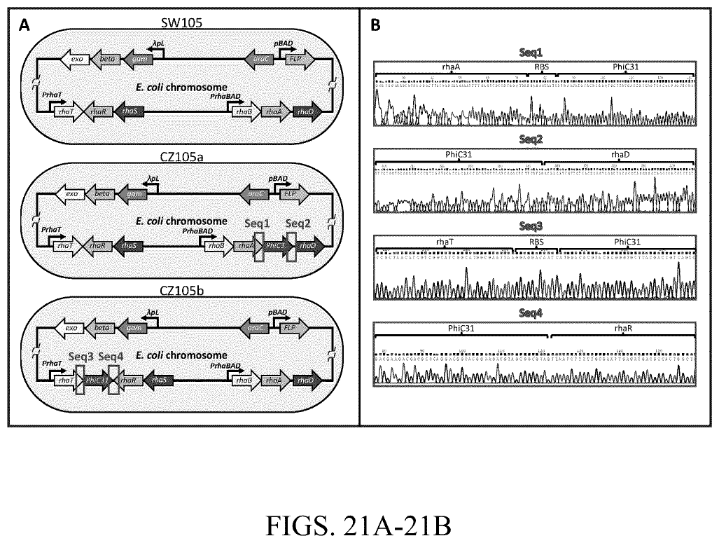

Two strains expressing PhiC31 driven by a rhamnose-inducible promoter were made by recombincering. Briefly, first, a positive selection and counterselection cassette, the RPSL-Amp operon in JMA1274, was amplified by PCR with primers, rhaTF and rhaTR, rhaA-Amp f and rhaA-Amp r, listed in Table 4. The resulting PCR products were purified and inserted immediately downstream of the rhaT gene or rhaA in the rhaBAD operon in the SW105 genome through positive selection for ampicillin resistance using the standard recombincering procedures. Second, PhiC31 (its 605 aa version) was amplified from pET11Phic31poly(A) (Addgene plasmid #18942) by PCR with primers, PhiC31 rhaF and PhiC31 rhaR3, rhaA-PhiC31 f and rhaA-PhiC31 r, listed in Table 4. The resulting PCR products were used to replace the RPSL-Amp operon that had previously been inserted into the SW105 genome through counterselection for streptomycin resistance and standard recombineering procedures, forming the rhaT-PhiC31 or rhaBA-PhiC31-D operon in the genome (FIGS. 21A-21B). PhiC31 and the corresponding flanking sequences in the E. coli genome were confirmed by Sanger sequencing.

### Construction of the Donor Vector Constructs.

DNA parts/transcription units. The DNA parts, including Arabidopsis thaliana U6-26 promoter and terminator (AtU6p and AtU6t), Solanum lycopersicum DFR (DIHYDROFLAVONOL-4-REDUCTASE) gene promoter (SIDFRP_{Spy(SIDFR)}), Cauliflower Mosaic Virus 35S promoter (35S), mCherry v1, 3xYpet, 3xNLS-EDLL, and Streptococcus pyogenes (Spy) sgRNA2.1 targeting SIDFRP_{Spy(SIDRF)} at the position-150 (sgRNA_{Spy(SIDFR)}-MS2), were directly amplified by PCR using iProof High-Fidelity DNA Polymerase (bio-rad.com) and subcloned into GoldenBraid entry vector, pUPD2. Templates and primers are listed in Table 5.

TABLE 5
Generation of the donor vector constructs.

| DNA parts and | | | |
|---|---|---|---|
| transcriptional units | Source/template | Cloning method | Primer sequence |
| pDGB3ω1_35S-MCP- | GB2085 | N/A | N/A |
| VPR-Tnos_35S- | | | |
| dCas9_{Spy}-EDLL-Tnos | | | |
| pUPD2_AtU6 | pHSN6A01 | domestication by | AtU6p-f, |
| promoter (AtU6p) | (addgene plasmid | PCR | GCGCCGTCTCGCTCGGGAGTCGACTT |
| (Part1) | #50586) | | GCCTTCCGCACAA (SEQ ID NO: 83) |
| | | | AtU6p-r2, |
| | | | GCGCCGTCTCGCTCACAATCACTACTT |
| | | | CGACTCTAG (SEQ ID NO: 84) |
| pUPD2_sgRNA_{Spy(SIDFR)}- | GB1437 | domestication by | crRNA-f3, |
| MS2 (Part2) | | PCR | GCGCCGTCTCGCTCGattGACTGGTTGG |
| | | | TGAGAGAAGA (SEQ ID NO: 85) |
| | | | gRNA linker, |
| | | | GACTGGTTGGTGAGAGAAGAgttttagagct |
| | | | agaaatagcaagttaaaataaggctag (SEQ |
| | | | ID NO: 86) |
| | | | tracrRNA-MS2 75-r, |
| | | | GCGCCGTCTCGCTCATTGCAAAAAAA |
| | | | AGGGAAGACTCCCCAG (SEQ ID NO: |
| | | | 87) |
| pUPD2_AtU6 | pHSN6A01 | domestication by | AtU6t-f2, |
| terminator (AtU6t) | (addgene plasmid | PCR | GCGCCGTCTCGCTCGGCAAAATTTTCC |
| (Part3) | #50586) | | AGATCGatttc (SEQ ID NO: 88) |
| | | | AtU6t-r, |
| | | | GCGCCGTCTCGCTCAAGCGTTATTGGT |
| | | | TTATCTCATCGGAACT (SEQ ID NO: 89) |
| pDGB3α1_AtU6p- | Part1 + Part2 + | GoldenBraid | N/A |
| sgRNA_{Spy(SIDFR)}-MS2- | Part3 | assembly | |
| AtU6t | | | |
| SIDFR promoter | GB0606 | domestication by | SIDFR f, |
| (pUPD2_SIDFRP_{spy(SIDFR)} | | PCR | GCGCCGTCTCGCTCGGGAGTGTTTGA |
| (Part 4) | | | ATGGATCCAGTTCG (SEQ ID NO: 90) |
| | | | SIDFR r2, |
| | | | GCGCCGTCTCGCTCAatggTTTCAGAAA |
| | | | TGAAAGGTAAAAAAGAG (SEQ ID NO: |
| | | | 91) |
| pUPD2_3x Ypet | DNA synthesis | domestication by | GB-3xYPet B2F, |
| (B2-B5) (Part 5) | | PCR | GCGCCGTCTCGCTCGCCATATGTCTAA |
| | | | GGGTGAAGAGTTG (SEQ ID NO: 92) |
| | | | GB-3xYPet B5R, |
| | | | GCGCCGTCTCGCTCAAAGCTTACTTGT |
| | | | AAAGTTCATTCATGCC (SEQ ID NO: 93) |
| pUPD2_Term35S#0 | DNA synthesis | domestication by | Term0 F, |
| (Part 6) | | PCR | GCGCCGTCTCActcgGCTTaaatcaccagtctct |
| | | | ctctacaaatc (SEQ ID NO: 94) |
| | | | GB-Term0_R, |
| | | | GCGCCGTCTCActcaAGCGctggattttggtttt |
| | | | aggaattag (SEQ ID NO: 95) |
| pDGB3α2_SIDFRp_{Spy(SIDFR)}- | Part4 + Part5 + | GoldenBraid | N/A |
| 3xYpet- | Part6 | assembly | |
| Term35S#0 | | | |
| 35S promoter | GB2085 | domestication by | 35S (A1-A3) Forward, |
| (pUPD2_35S) (Part 7) | | PCR | GCGCCGTCTCGCTCGGGAGACTAGAG |
| | | | CCAAGCTGATCTC (SEQ ID NO: 96) |
| | | | 35S-r, |
| | | | GCGCCGTCTCGCTCAATGGCAGCGTG |
| | | | TCCTCTCCAAATGaaatgaac (SEQ ID NO: |
| | | | 97) |
| pUPD2_dCas9_{Sth} | pDE-St1_Cas9 | domestication by | StdCas9 f (B2), |
| (Part8) | | PCR | GCGCCGTCTCGCTCGCCATGcccaagaaga |
| | | | agaggaaggtgTCTGATCTCGTGCTCGGA |
| | | | (SEQ ID NO: 98) |
| | | | StdCas9 r (B4), |
| | | | GCGCCGTCTCGCTCACGAACCGAAAT |
| | | | CGAGCTTAGGCTTATC (SEQ ID NO: 99) |
| | | Inverse PCR | StdCas9 D9A f, |
| | | | CTCGGACTCGcTATCGGAATCGGATCT |
| | | | GTTG (SEQ ID NO: 100) |
| | | | StdCas9 D9A r, |
| | | | ATTCCGATAgCGAGTCCGAGCACGAG |
| | | | ATCA (SEQ ID NO: 101) |
| | | Inverse PCR | StdCas9 H599A f, |
| | | | GAAGTGGATgctATCCTCCCACTCTCAA |
| | | | TCAC (SEQ ID NO: 102) |
| | | | StdCas9 H599A r, |
| | | | TGGGAGGATagcATCCACTTCGAACTG |
| | | | GTTAG (SEQ ID NO: 103) |
| pUPD2_3xNLS-EDLL | GB2085 | domestication by | 3xnls-EDLL f (B5), |
| (Part9) | | PCR | GCGCCGTCTCGCTCGTTCGGATCCTAA |
| | | | AAAGAAGCGA (SEQ ID NO: 104) |
| | | | 3xnls-EDLL r (B5), |
| | | | GCGCCGTCTCGCTCAAAGCTCAGCGT |
| | | | TTGCGTTCTT (SEQ ID NO: 105) |
| pDGB3α1_35S- | Part7 + Part8 + | GoldenBraid | N/A |
| dCas9_{Sth}-3xNLS- | Part9 | assembly | |
| EDLL-Term35S#0 | | | |
| pEGB1α2_Pnos:NptII: | GB0184 | N/A | N/A |
| Tnos | | | |
| Part10a | overlapping | PCR | 75 nt St tracrRNA f, |
| | primers | | GCGCCGTCTCGCTCGattGAATCTTGCA |
| | | | GAAGCTACAAAGATAAGGCTTCATGC |
| | | | CGAA (SEQ ID NO: 106) |
| | | | St tracrRNA-long r, |
| | | | cgAAAACACCCTGCCATAAAATGACA |
| | | | GGGTGTTGATTTCGGCATGAAGCCTT |
| | | | ATCT (SEQ ID NO: 107) |
| Part10b | Part10a | PCR | mature St tracrRNA f, |
| | | | GCGCCGTCTCGCTCGattGCAGAAGCTA |
| | | | CAAAGATAAG (SEQ ID NO: 108) |
| | | | St tracrRNA r, |
| | | | CACCCTGCCATAAAATGACA (SEQ ID |
| | | | NO: 109) |
| Part10c (scoffold) | Part10b | PCR | St gRNA scaffold f, |
| | | | gtctttgtactctggtaccagaagctacaaagat |
| | | | aagg (SEQID NO: 110) |
| | | | Sth tracrRNA-short r, tgtcattttatg |
| | | | gcagggtg (SEQ ID NO: 111) |
| Part10d (MS2) | GB1437 | PCR | Sth tracrRNA-short-MS2 f, |
| | | | tgtcattttatggcagggtggggagcacatgagg |
| | | | aatcac (SEQ ID NO: 112) |
| | | | tracrRNA-MS2 75 r, |
| | | | ctggggagtcttcccTTTTTTTTGCAATGAGCG |
| | | | AGACGGCGC (SEQ ID NO: 113) |
| pUPD2_sgRNASth(ADH1)- | Part10c + | overlapping PCR | St gRNA ADH1f, |
| MS2 (Part 10) | Part10d | | GCGCCGTCTCGCTCGattGAAGTGGAGG |
| | | | TTGCTCCACCgtctttgtactctggtacca |
| | | | (SEQ ID NO: 114) |
| | | | tracrRNA-MS2 75 r, |
| | | | ctggggagtcttcccTTTTTTTTGCAATGAGCG |
| | | | AGACGGCGC (SEQ ID NO: 115) |
| pDGB3α1_AtU6p- | Part1 + Part10 + | GoldenBraid | N/A |
| sgRNASth(ADH1)-MS2- | Part3 | assembly | |
| AtU6t: | | | |
| pUPD2_SIDFRp_{Sth(ADH1)} | pUPD2_SIDFRp | inverse PCR | SIDFR_ADH1 f, |
| (Part11) | | | tctgcGGTGGAGCAACCTCCACTTCacatttg |
| | | | gttaatccaaccaag (SEQ ID NO: 116) |
| | | | SIDFR_ADH1 r, |
| | | | TGGAGGTTGCTCCACCgcagaaagctagtgtga |
| | | | agtgtctggc (SEQ ID NO: 117) |
| pUPD2_mCherry v1 | DNA synthesis | domestication by | mCherry_f, |
| (B2-B5) (Part12) | | PCR | GCGCCGTCTCGCTCGCCATGGTGAGT |
| | | | AAGGGTGAAGAAG (SEQ ID NO: 118) |
| | | | mCherry_r, |
| | | | GCGCCGTCTCGCTCAAAGCTCAATAG |
| | | | AGCTCATCCATACC (SEQ ID NO: 119) |
| pDGB3α2_SIDFRp_{Sth(ADH1)}- | Part11 + | GoldenBraid | N/A |
| mCherry- | Part12+ | assembly | |
| Term35S#0 | Part6 | | |
| pDASH-DII-α1_FAST | pDGE347_FAST_ | domestication by | FAST f1, |
| | Bar_pRPS5a: | PCR | GCGCgGTCTCGGGAGACTAAATGGAG |
| | cas9_ccdB | | CAACCTACTG (SEQ ID NO: 120) |
| | (addgene | | FAST r1, |
| | plasmid #153228) | | GCGCgGTCTCGCATCTCCTTTCTTAAT |
| | | | ATATCTAACA (SEQ ID NO: 121) |
| | | | FAST f2, |
| | | | GCGCgGTCTCGGATGAGAAGTCACGT |
| | | | GTCAAT (SEQ ID NO: 122) |
| | | | FAST r2, |
| | | | GCGCgGTCTCGagcgGTTCTAGAATGTC |
| | | | GCGGAAC (SEQ ID NO: 123) |
| pDASH-DIIS-α1_ | pDGB3α1_AtU6p2_ | BsmB I | N/A |
| sfGFP | sfGFP | digestion/ligation | |

To generate the Streptococcus thermophilus Stl (Sth) dCas9-based CRISPRa transcriptional unit, the original protospacer and the Spy PAM site (GACTGGTTGGTGAGAGAAGAagg (SEQ ID NO:144), where small letters indicate the Spy PAM site) at the position-150 of SIDFRP_{Spy(SIDFR)} in pUPD2 were replaced by the ADH1 protospacer followed by the Sth PAM sequence (GAAGTGGAGGTTGCTCCACCgcagaaa (SEQ ID NO: 145), where small letters indicate the Sth PAM site) via site-directed mutagenesis, according to QuickChange Site-Directed Mutagenesis kit's manual (agilent.com), producing the pUPD2_SIDFRP_{Sth(ADH1)} DNA part. Similarly, the Arabidopsis codon-optimized Sth Cas9 orthologue was first amplified from pDE-St1_Cas9 and subcloned into pUPD2, from which, dead Sth Cas9, dCas9_{Sth} [St1 dCas9 (D9A, H599A)] was generated by two rounds of inverse PCR with primers, StdCas9 D9A f and StdCas9 D9A r, StdCas9 H599A f and StdCas9 H599A r (Table 5). Sth engineered sgRNA (v1)-MS2, sgRNA_{Sth(ADH1)}-MS2, was created by overlapping PCR using primers, St gRNA scaffold f, Sth tracrRNA-short r, St gRNA ADH1f, Sth tracrRNA-short-MS2 f, and tracrRNA-MS2 75 r (Table 5).

To generate the pDASH-DII-α1_FAST transcriptional unit, the FAST cassette was domesticated from pDGE347_FAST_Bar_pRPS5a: Cas9_ccdB into the donor vector pDASH-DII-α1 according to GoldenBraid assembly to remove an internal BsmBI site. To generate the pDASH-DIIS-α1_sfGFP, the pDGB3alpha1_AtU6p2_sfGFP transcriptional unit (lab collection) was digested with BsmBI, releasing two fragments (backbone and AtU6p2_sfGFP cassette). The small fragment containing sfGFP cassette was purified and ligated with the DASH donor vector pDASH-DIIS-α1 pre-digested with the same enzyme. All sequences were confirmed by Sanger sequencing.

Golden Gate Assembly. For the assembly of DNA parts into transcription units (TU) and individual TUs into modules, GoldenBraid assembly was performed according to NEB's Golden Gate (24 fragment) Assembly Protocol (neb.com).

### PhiC31-Mediated Integration and FLP-Mediated Excision Procedure

A single colony transformed with the acceptor (pDASH-AIK) and donor vectors was grown in 2 ml of liquid LB media supplemented with kanamycin and spectinomycin or ampicillin (depending on which donor vector was used) in a 15-ml sterile plastic culture tube at 30° C. overnight. 20 μl of overnight culture were used to inoculate 1 ml of liquid LB media supplemented with antibiotics for the acceptor and donor vector selection. When OD₆₀₀ reached ˜0.6, 10 μl of 10% sterile L-rhamnose were added to the cell culture to induce PhiC31 and incubated for 3 hours at 30° C. E. coli cells were harvested by brief centrifugation and re-grown in 1 ml of liquid media supplemented with kanamycin and 10 μl of 10% sterile L-arabinose to induce FLP and incubated for 3 hours at 30° C. The culture was then re-streaked for single colonies on an LB plate supplemented with kanamycin. The plate was incubated at 30° C. for 2 days for single colony selection and analysis.

### Agrobacterium and Plant Transformation

The pDASH-AIK-Big construct was transformed into Agrobacterium tumefaciens GV3101 by electroporation. After 2 h incubation in plain LB at 28° C. to allow the cells to recover and express the antibiotic-resistance genes, the electroporated cells were spread for single colonies on LB plates supplemented with kanamycin/gentamycin/rifamycin. Colonies were confirmed by colony PCR [by checking all junctions between two CRISPRa copies and LacZ, FAST, and sfGFP] with primers listed in Table 6 and used in transient expression assays in N. benthamiana leaves (by infiltrating the leaves with Agrobacterium suspension with OD₆₀₀ of 0.1) or for Arabidopsis transformation via the floral dip method.

TABLE 6
Primers used to check the integrity of the pDASH-AIK-Big construct.

| Primer name | Primer sequence | Position checked |
|---|---|---|
| P27 2nd | CAAATTGACGCTTAGACAAC (SEQ ID NO: 124) | left junction of the acceptor |
| | | vector |
| MCP r | GCGCCGTCTCGCTCACGAACCATAAATACCAGAA | |
| | TTAGCAGCAATAG (SEQ ID NO: 125) | |
| mCherry f | GCGCCGTCTCGCTCGCCATGGTGAGTAAGGGTGA | right junction of the acceptor |
| | AGAAG (SEQ ID NO: 126) | vector |
| P28 | CGGAGAATTAAGGGAATTACC (SEQ ID NO: 127) | |
| MF_YPet-3′_F | CGAAGGCATGAATGAACTTTACAAGTAA (SEQ ID | Junction between MD5 and |
| | NO: 128) | MD6 |
| StdCas9 D9A r | ATTCCGATAgCGAGTCCGAGCACGAGATCA (SEQ | |
| | ID NO: 129) | |
| mCherry f | GCGCCGTCTCGCTCGCCATGGTGAGTAAGGGTGA | Junction between MD6 and |
| | AGAAG (SEQ ID NO: 130) | right border (RB) |
| pDGB3 r | CGGATAAACCTTTTCACGC (SEQ ID NO: 131) | |
| Term0 F | GCGCCGTCTCActcgGCTTaaatcaccagtctctctctacaaa | Junction between MD7 and |
| | tc (SEQ ID NO: 132) | MD9 |
| FAST r2 | GCGCgGTCTCGagcgGTTCTAGAATGTCGCGGAAC | |
| | (SEQ ID NO: 133) | |
| pDGB3 f | GTATCGAGTGGTGATTTTGTGC (SEQ ID NO: 134) | junction between left border |
| | | (LB) and MD7 |
| MCP r | GCGCCGTCTCGCTCACGAACCATAAATACCAGAA | |
| | TTAGCAGCAATAG (SEQ ID NO: 135) | |
| CPEC2_alpha1 f | AGCGATGAGTAGAGACCCACAGCTTGTCTG (SEQ | junction between LacZ and |
| | ID NO: 136) | MD7 |
| MCP r | GCGCCGTCTCGCTCACGAACCATAAATACCAGAA | |
| | TTAGCAGCAATAG (SEQ ID NO: 137) | |
| FAST f2 | GCGCgGTCTCGGATGAGAAGTCACGTGTCAAT | junction between FAST and |
| | (SEQ ID NO: 138) | MD7 |
| MCP r | GCGCCGTCTCGCTCACGAACCATAAATACCAGAA | |
| | TTAGCAGCAATAG (SEQ ID NO: 139) | |
| sfGFP 1180f | GTCCTGCTGGAATTTGTGAC (SEQ ID NO: 140) | juntion between sfGFP and |
| | | MD7 |
| MCP r | GCGCCGTCTCGCTCACGAACCATAAATACCAGAA | |
| | TTAGCAGCAATAG (SEQ ID NO: 141) | |
| mCherry f | GCGCCGTCTCGCTCGCCATGGTGAGTAAGGGTGA | juntion between MD7 and RB |
| | AGAAG (SEQ ID NO: 142) | |
| pDGB3 r | CGGATAAACCTTTTCACGC (SEQ ID NO: 143) | |

N. benthamiana (the laboratory accession, lab) seeds were sterilized using 50% (v/v) ethanol and directly sown onto soil (Sungro horticulture professional grow mix mixed 1:1 with Jolly gardener Pro-line C/B growing mix) for germination. At one-week-old stage, seedlings were transplanted to 24-cell nursery flats, one plant per cell, and grown at 22° C. under a 16 h-light/8 h-dark cycle in a growth chamber with a light intensity of ˜120 μmol/m²/s.

Similarly, Arabidopsis thaliana (Col-0) seeds were sterilized using 50% (v/v) bleach solution and kept at 4° C. for three days (for stratification), then directly sown onto soil described above with same growth conditions as for N. benthamiana.

### Microscopy to Assess Ypet and Mcherry Fluorescence in Plants.

Samples were imaged using the Zeiss Axio Imager M2 upright microscope with a 5× objective (N.A. Immersion 0.16 and type Plan APO) and the corresponding multiple fluorescence channels, which were controlled by the Zeiss ZEN (blue edition) software. The Keyence BZ-X80 microscope was used with the 10× objective (N.A. Immersion 0.45 and type Plan APO). Three channels (Fluor GFP, Fluor TxRed, and BF/phase) were used to capture Ypet and mCherry fluorescence and bright field images, respectively. Images were exported as TIFF files.

### 6. EXAMPLES

Manipulating gene expression using transgenic approaches is at the core of gene functional studies and a key element in biotechnological applications. The generation of the DNA constructs needed for these types of transgenic experiments can be significantly streamlined and accelerated using efficient synthetic biology DNA assembly tools. Among them, Golden Gate-based platforms have gained significant popularity due to their efficiency, experimental simplicity, and ability to utilize standardized, reusable DNA parts. One important limitation of these assembly technologies is that their efficiency greatly decreases as the size of the DNA parts being assembled increases, with a practical upper limit of about 25 kb, making the generation of relatively complex multigenic DNA constructs challenging. This could be a significant drawback as the demand for large DNA constructs to precisely control multigenic complex traits continues to increase in both synthetic biology and biotechnology applications. To specifically address this size limitation, highly efficient recombination-based gene stacking systems such as the GAANTRY and TGSII have been developed. While solving the size problem, the gene-stacking process in these systems imposes certain constraints. For instance, although the orientation and order of the genes in the final construct can be designed at will, once a gene is added, it cannot be removed, replaced, or modified without requiring a new assembly. Furthermore, none of these stacking systems are fully integrated with the popular Golden Gate-based platforms, resulting in a loss of the efficiency, standardization, and reusability benefits these platforms typically offer.

The DASH system was developed to overcome the shortcomings of existing DNA assembly and gene-stacking platforms while retaining their strengths. Thus, the DASH system combines the efficiency and experimental simplicity of the Golden Gate-based technologies with the large cargo capacity of recombination-based methods while offering the sequence modification flexibility characteristic of recombineering. To achieve this, the recombineering strain SW105, the GoldenBraid alpha and omega vectors, and the large-capacity TAC vector pYLTAC17 were all reengineered. The SW105 E. coli recombincering strain has an arabinose-inducible FLP recombinase and a very tight temperature-inducible recombinase cassette comprising the beta, gam, and exo lambda phage genes. To enable this strain to perform unidirectional integration between two plasmids, the PhiC31 integrase capability was incorporated, which enables the unidirectional recombination between two heterotypic sites in vivo with high efficiency. Other platforms that also rely on similar integrases to catalyze the recombination between two plasmids in vivo, such as the GAANTRY, avoid possible undesirable effects associated with the expression of these recombinases by transiently expressing them from a helper vector. To minimize the number of elements in the systems described herein and, at the same time, ensure tight regulation in the expression of PhiC31, the coding sequence of this gene was placed under the control of rhamnose-inducible operons in the E. coli genome of the SW105 recombincering strain. One potential issue with this strategy, compared to those that express the recombinase from a plasmid, is that, in this case, a single copy of PhiC31 driven by an endogenous E. coli promoter was used. This raises the question of whether the expression levels will be sufficient to trigger efficient recombination between plasmids in vivo. With this potential problem in mind, the PhiC3/CDS was placed under the control of two different endogenous rhamnose-inducible promoters, with the idea that at least one of them would provide a sufficient level of expression to trigger the integration but not so high to result in toxic, detrimental effects. Of the two strains generated, CZ105a demonstrates normal growth and high efficiency of rhamnose-inducible PhiC31, making it a suitable choice for the DASH system.

The GoldenBraid pDGB3 vectors were chosen as the foundation for the DASH system's assembly system because they provide a straightforward and efficient method to combine sets of DNA parts and pairs of transcriptional units or modules reiteratively. Importantly, standardized DNA parts and assembled transcriptional units from various other Golden Gate-based platforms using BsaI as the cloning enzyme can be easily transferred to the DASH alpha donor vectors and, therefore, used in gene stacking reactions with the pDASH acceptor vector. Thus, for example, standardized DNA parts from GoldenBraid, Loop, Mobius, and MoClo, and assembled transcription units from GoldenBraid, Loop, Mobius, and Start-Stop can be directly transferred to a DASH alpha donor vectors. In addition, relatively simple modifications would be needed to create the DASH shuttle vectors or add compatibility linkers that allow for even greater integration of other type IIS restriction enzyme-based platforms, as done for MoClo (FIGS. 17A-17B) and could be easily done in a similar fashion for other systems such as GreenGate, JMC, etc. (FIG. 16). Finally, the developers of the GoldenBraid platform have continued to update the tools and expand the collection of plant parts that the DASH system can also leverage.

Similarly, the pYLTAC17 vector was used as the backbone for the DASH acceptor vector due not only to its high-cargo capacity but also because it has been used extensively in recombincering experiments where plant genomic sequences harbored in a TAC are modified in E. coli and then transferred into plants via Agrobacterium-mediated transformation. Although the efficiency of plant transformation may not match the high levels achieved with standard binary vectors for small DNA constructs or the pRi Agrobacterium plasmid for larger constructs, it likely represents the best compromise when working with large DNA constructs that may require post-assembly modifications. This ability to propagate this binary vector in E. coli represents a significant advantage over the native pRi plasmid, which can only be propagated in Agrobacterium. Furthermore, results showed that the pYLTAC17-derived pDASH-AIK vector can be used in N. benthamiana transient expression assays, broadening the downstream applications of the DASH system. Finally, although a significant number of truncations of large T-DNAs inserted in the plant genome was observed in the past, the simplicity by which two different selectable markers (kanamycin and FAST, for example) could be added at the beginning and end of the T-DNA should aid in the identification of plants carrying the complete insert.

As mentioned above, the basic DASH system consists of a single E. coli strain, four donor vectors, and one acceptor vector, reflecting the platform's simplicity. Seven additional vectors were developed to facilitate specific experimental pipelines. Thus, for example, a second acceptor vector, pDASH-AIIK (FIGS. 17A-17B), with an attB^{CC} instead of the attP^{TT} site, is also available, allowing the direct stacking of DII-type DASH donor vector and eliminating the need of an extra assembly reaction to move the cargo from a DII to a D1 vector. Similarly, to assist in the removal of the donor vectors' backbones after the integration and excision recombination reactions, a set of four donor vectors expressing the SacB counter-selectable marker gene has been generated (FIG. 17B). SacB is toxic to cells grown in the presence of sucrose and can be leveraged to get rid of the donor plasmid post-integration and backbone excision step, thus helping to bypass time-consuming plasmid re-transformation or re-streaking steps and colony-PCR screens. This eliminates the need to ‘curate’ the final construct through plasmid segregation by re-transforming, and instead involves simply growing the cells with the final construct in the presence of sucrose. Finally, to illustrate how simple modifications of the DASH system could be used to expand the compatibility of the DASH platform, the pDASH-DII-α1 (MoClo) and pDASH-DI-α2 (MoClo) were developed to allow for the transfer of transcriptional units from the MoClo level M plasmids to the DASH system.

One significant limitation of all current sequential DNA assembly and stacking systems is that any alteration, even a single nucleotide change, generally requires going back in the assembly chain and restarting from the point where the part necessitating modification was included. This obviously becomes especially critical in synthetic biology and modern plant biotechnological applications that often require relatively large multigenic constructs and several iterations of the “design, build, test, and learn” cycle. Recombineering represents an excellent post-assembly modification approach as it can deal with genome-size DNA molecules and generate variable-size insertions or deletions, from single nucleotide to several thousand base pair. Obviously, any non-toxic DNA molecule that can be propagated in E. coli, and any recombincering-compatible bacterial strains, including Agrobacterium, can be modified by recombincering. Replacing sfGFP with an RPSL-Amp cassette in the ˜100 kb construct demonstrates that integrating recombineering into the DASH platform simplifies post-assembly modifications, even for very large DNA constructs.

Finally, the DASH system was developed with the idea of providing a user-friendly platform that, although capable of handling very large DNA molecules, does not require sophisticated laboratory techniques or users highly skilled in molecular biology or microbiology. The simplicity of the system is evident in the six elements that comprise it: the CZ105a E. coli strain, four donor vectors, and one acceptor vector. The simplicity of the experimental pipeline is demonstrated by the initial assembly of DNA components, which employs the classical Golden Gate-based single-tube restriction/ligation cloning reaction. Additionally, the stacking process requires only the preparation of competent cells harboring the acceptor vector, electroporation of the donor vector, induction of the desired recombinase upon the addition of the appropriate sugar, and selection of the recombinants with the proper antibiotic-supplemented media (FIG. 25). It was anticipated that the DASH system's simplicity and ease of use, along with its high efficiency, robustness, and compatibility with various Golden Gate-based systems, combined with the increasing availability of GoldenBraid and other DASH-compatible tools and DNA parts, will make this platform appealing to a diverse group of plant biologists, particularly those who need to generate large DNA constructs or introduce post-assembly modifications.

The accompanying Examples are offered as illustrative as a partial scope and particular embodiments of the disclosure and are not meant to be limiting of the scope of the disclosure.

### Example 1: Modifying E. coli Strain

In the SW105 recombincering strain of E. coli, part of phage λ genome (from cl to int, including gam, beta, and exo) is integrated into the host's genome. The right side of the prophage from cro through attR, including the host's bioA, was replaced by the araC-P_{BAD}-flpe cassette (see Lee et al., 2001 for more details). As a result of these genome modifications, the SW105 genome contains temperature-inducible gam, beta, and exo (that jointly catalyze homologous recombination) and an arabinose-inducible recombinase FLP (that recognizes FRT sequences). As shown in FIGS. 21A-21B, c1857 is a temperature sensitive (heat-inducible) λ repressor that controls the expression of gam, beta and exo, and AraC is an E. coli arabinose-regulated transcription factor that controls the expression of FLP recombinase.

To express PhiC31 in a tightly regulated manner, another stimulus-inducible promoter was employed. At least 12 promoter-regulator pairs that operate orthogonally have been reported in E. coli (see Schuster and Reisch, 2021 NAR). Since FLP in SW105 is induced by arabinose, another sugar, rhamnose, was chosen to control the expression of PhiC31. PhiC31 was inserted into the E. coli SW105 genome, taking advantage of endogenous rhamnose-inducible promoters (instead of making a construct expressing PhiC31 driven by a rhamnose-inducible promoter). FIGS. 21A-21B shows the gene cluster for rhamnose metabolism in the E. coli genome. Two rhamnose-inducible promoters, PrhaBAD and PrhaT, have been reported. PrhaBAD drives the rhaBAD operon, while PrhaT drives rhaT, and the two promoters are repressed by RhaS and RhaR proteins, respectively.

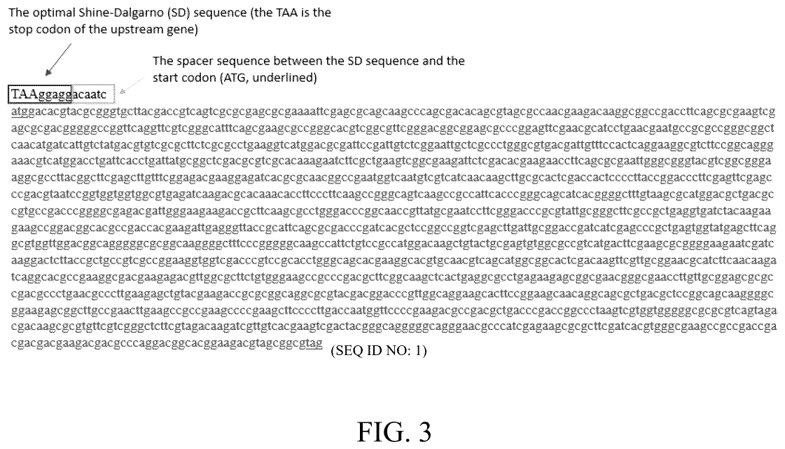

To make CZ105, PhiC31 with a partial ribosome binding site at the 5′ end was inserted by recombineering either immediately downstream of rhaA in the rhaBAD operon (strain CZ105a) or immediately downstream of rhaT to form an operon with rhaT (strain CZ105b) (FIGS. 21A-21B). PhiC31 (FIG. 3) with partial Shine-Dalgarno (SD) sequence (ggagg) and spacer sequence (acaatc) at its 5′ end was amplified by PCR and inserted into the E. coli SW105 genome by recombincering. After integration, the partial SD sequence and the stop codon (TAA) of the upstream gene formed the optimal SD sequence.

### Example 2: Modifying the Acceptor Vector (attP^{TT}-FRT Sequence)

To build an acceptor vector, a JATY transformable BAC pYLTAC (aka TAC) vector was used as a backbone and its T-DNA region was modified to make it suitable for recombinase-mediated DNA integration (to enable integration of any components needed at any steps). An attP^{TT} site was included to be used for the integration of a donor vector of interest and an FRT site was included for the removal of the donor vector backbone.

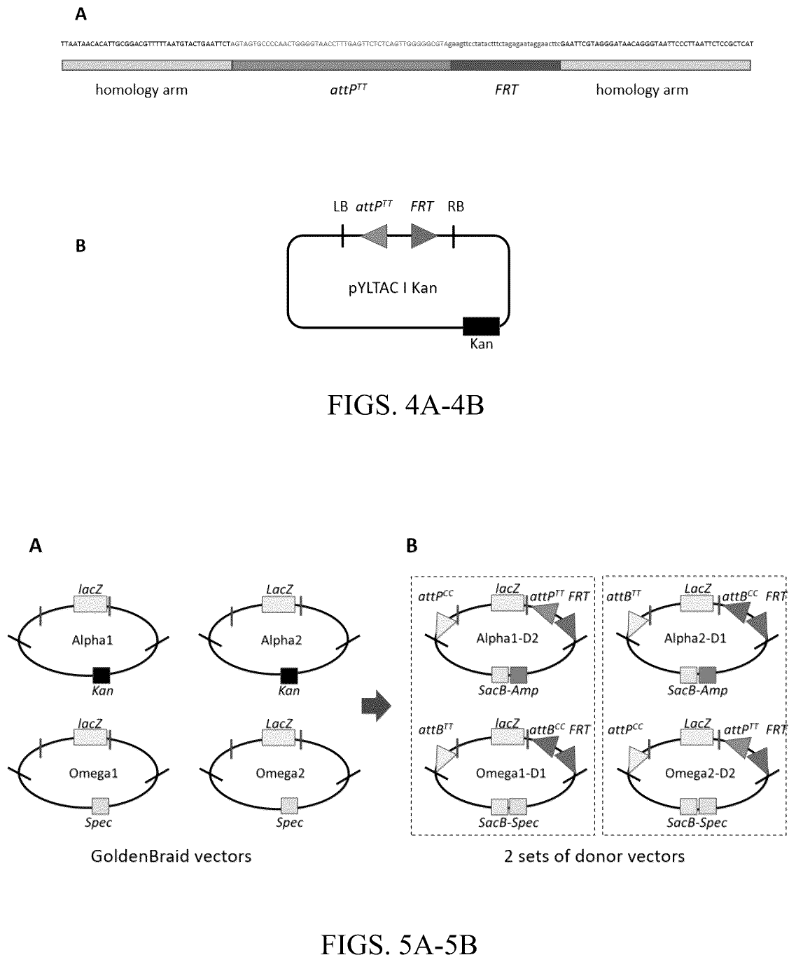

A fragment containing an attP^{TT} and FRT sites flanked by ˜40 nt arms of homology to the JATY vector sequence (FIG. 4A) was generated by PCR and used for homologous recombination into the JATY clone by recombineering. The resulting T-DNA region in the acceptor vector (FIG. 4B) contains an attP^{TT} and a FRT site flanked by ˜50 nt of original T-DNA sequences. These two flanking regions can be used as docking sites for further vector modification if needed.

### Example 3: Modifying Donor Vectors

Two alpha vectors and two omega vectors that are components of the standard GoldenBraid molecular cloning system (FIG. 5A) were modified to produce two sets of donor vectors shown in dashed rectangles (FIG. 5B). Each set consists of Donor 1 (D1, D1) and Donor 2 (D2, DII) with different antibiotic genes so that there is one Donor 1 in alpha and one Donor 1 in omega vector.

For each vector, an att site was inserted upstream of lacZ selectable marker (between LB marked by the black line and BsaI/BsmBI sites marked by the red line). This att will be used for integration. Downstream of lacZ, the second att site and an FRT site were inserted between BsaI/BsmBI sites (red line) and RB (black line). The FRT is needed for the removal of the donor vector backbone, while the second att is necessary for the integration of the second donor vector.

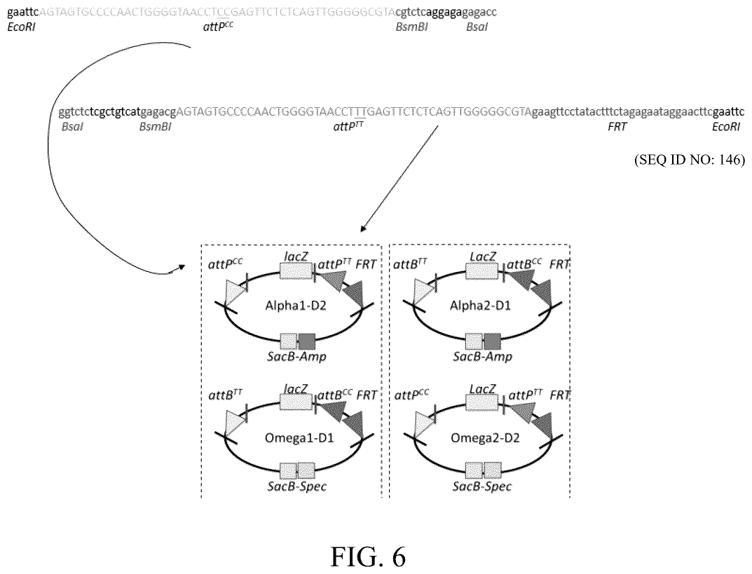

Specifically, to make Alpha1-D2, PCR fragments containing attP^{CC} and attP^{TT}_FRT were inserted between EcoRI and BsaI, BsaI and EcoRI sites, respectively, in the original GoldenBraid pDGB3alpha1 vector (FIG. 6).

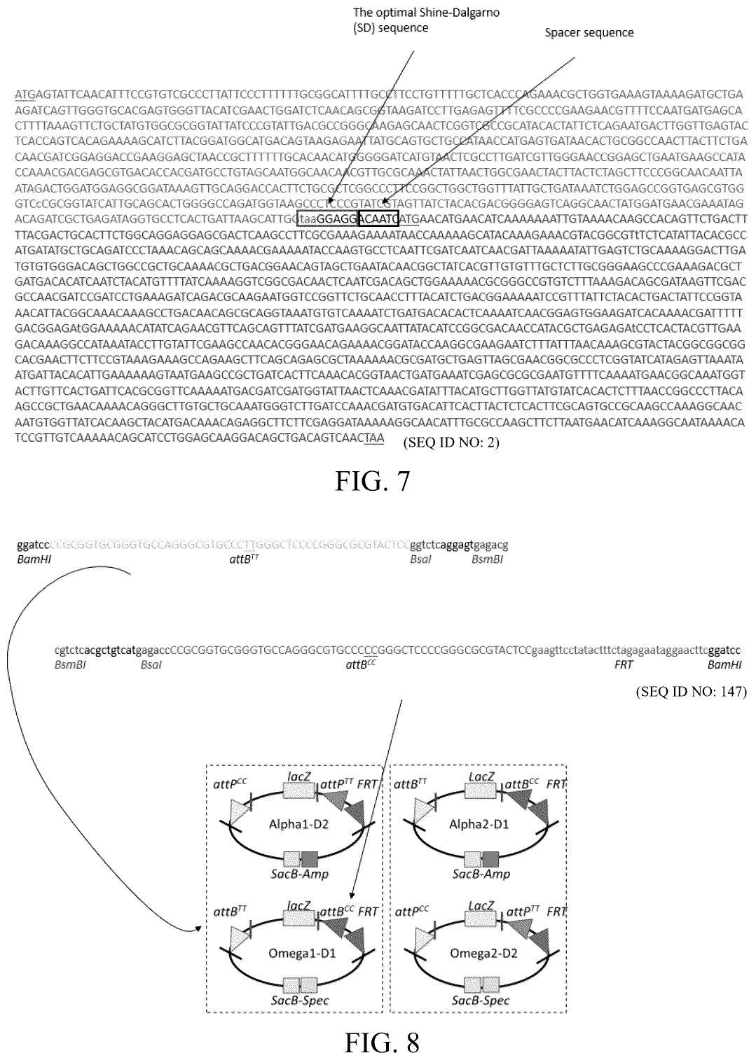

In addition, for alpha vectors, Kan was replaced by Amp to make these donor vectors compatible with the Kan-resistant acceptor vector. Furthermore, partial Shine-Dalgarno sequence (GGAGG) and spacer sequence (ACAATC) followed by SacB was inserted downstream of the stop codon of the Ampicillin (Amp) or the spectinomycin (Spec) gene to allow for vector counter-selection in the presence of sucrose (sucrose in the media is toxic to SacB-containing clones). The domesticated ampicillin (Amp) resistance and SacB genes were linked into one operon by the optimal SD sequence and a spacer sequence, which was used to replace the original kanamycin (Kan) resistance gene (FIG. 7).

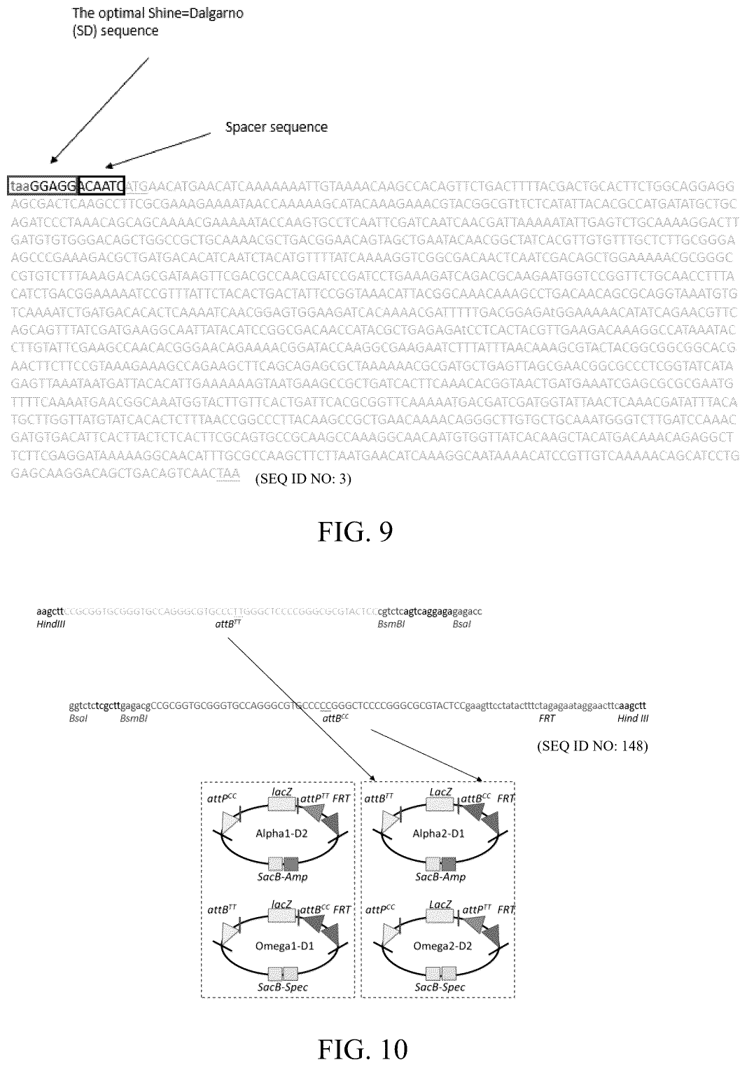

To make Omega1-D1, PCR fragments containing attB^{TT} and attB^{CC}-FRT were inserted between BamHI and BsaI, BsaI and BamHI sites, respectively, in the original GoldenBraid pDGB3omega1 vector (FIG. 8). In addition, partial Shine-Dalgarno sequence (GGAGG) and spacer sequence (ACAATC) followed by SacB gene was inserted downstream of the stop codon of the Spec marker to enable counter-selection (FIG. 9).

To generate Alpha2-D1, PCR fragments containing attB^{TT} and attB^{CC}-FRT were inserted between HindIII and BsmBI, BsmBI and HindIII sites, respectively, in the original GoldenBraid pDGB3alpha2 vector (FIG. 10). In addition, the original Kan gene was replaced with Amp-SacB positive-negative dual selectable marker.

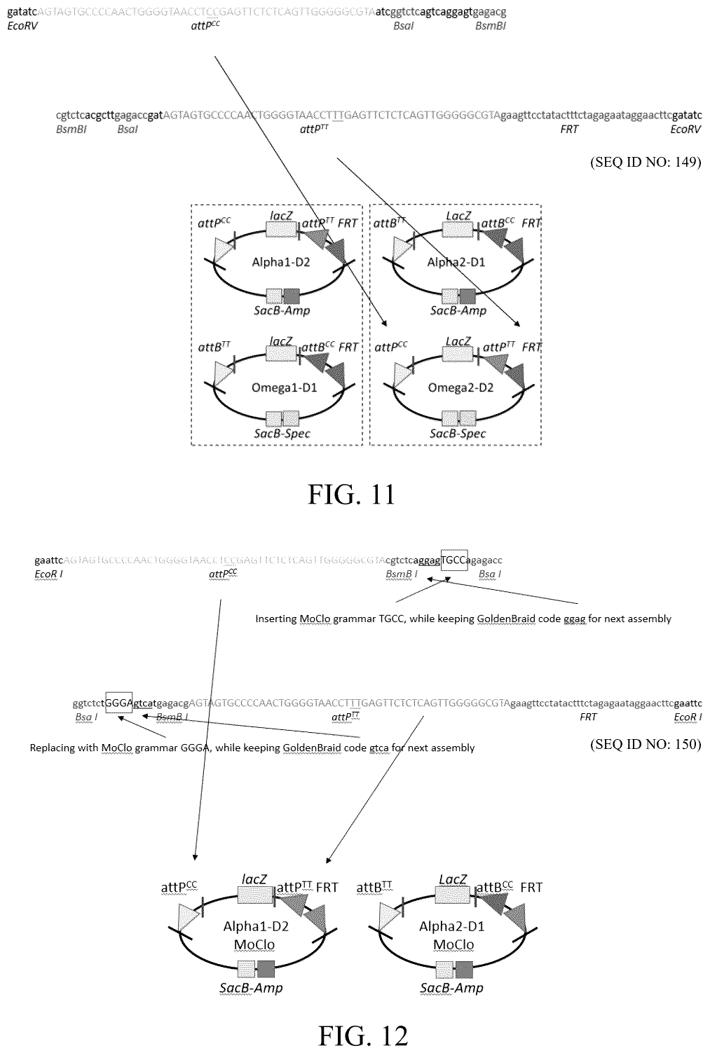

To make Omega2-D2, PCR fragments containing attP^{CC} and attP^{TT}-FRT were inserted into the two EcoRV sites (that flank the BsaI sites and lacZ) in the original GoldenBraid pDGB3omega2 vector (FIG. 11). This subcloning regenerates intact EcoRV sites upstream of attp^{CC} and downstream of attP^{TT}_FRT in their original positions. In addition, partial Shine-Dalgarno sequence (GGAGG) and spacer sequence (ACAATC) followed by SacB was inserted downstream of Spec.

To make the system compatible with MoClo, a duo of MoClo-specific grammar codes, TGCC and GGGA, were introduced into GoldenBraid alpha1 and alpha2 vectors (FIG. 12). To make Alpha1-D2 MoClo, TGCC was inserted and GGGA was used to replace the original GoldenBraid grammar, cgct, so that when the vector is digested by BsaI, it can accept DNA fragment(s) with grammar TGCC and GGGA from MoClo. Importantly, the original GoldenBraid grammar codes, ggag and gtca, in Alpha1 were kept intact so that the construct can be assembled according to GoldenBraid system. In addition, partial Shine-Dalgarno sequence (GGAGG) and spacer sequence (ACAATC) followed by SacB was inserted downstream of the stop codon of Amp.

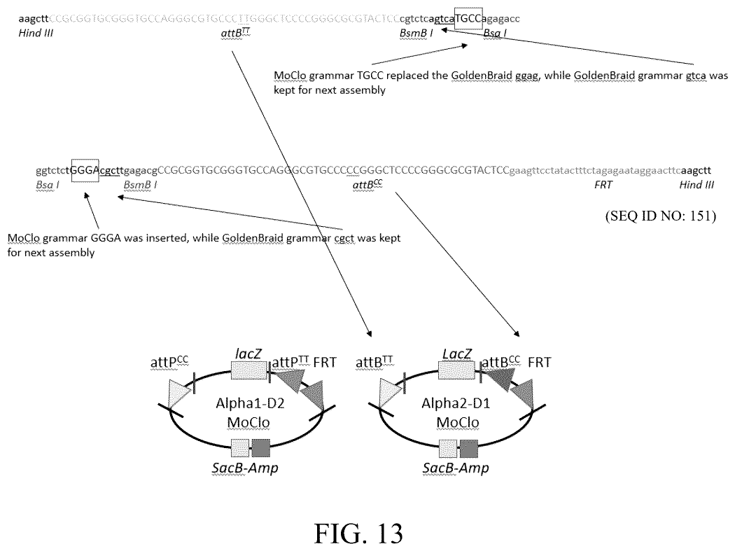

Similarly, to make Alpha2-D1 MoClo, MoClo grammar codes TGCC and GGGA were introduced for BsaI digestion, and GoldenBraid grammar gtca and cgct were kept for BsmBI digestion for next assembly (FIG. 13).

### Example 4: Assembly of 8 Transcriptional Units (TUs) in a Donor Vector

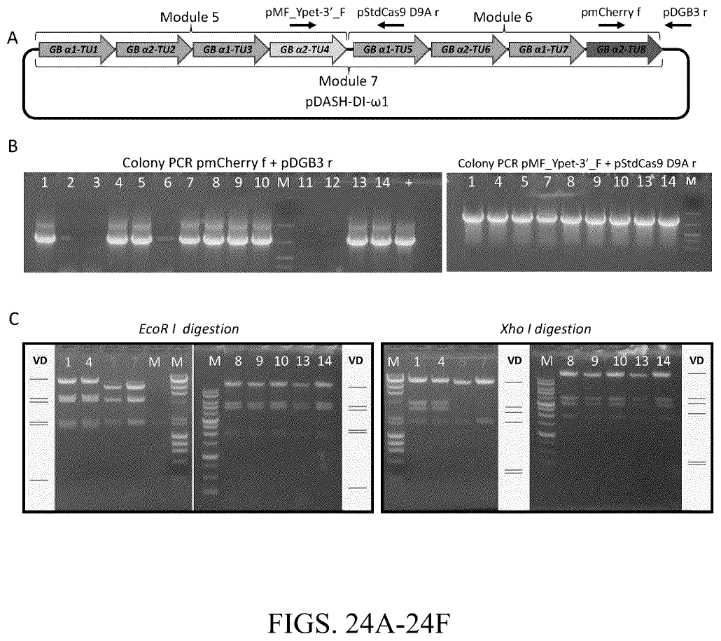

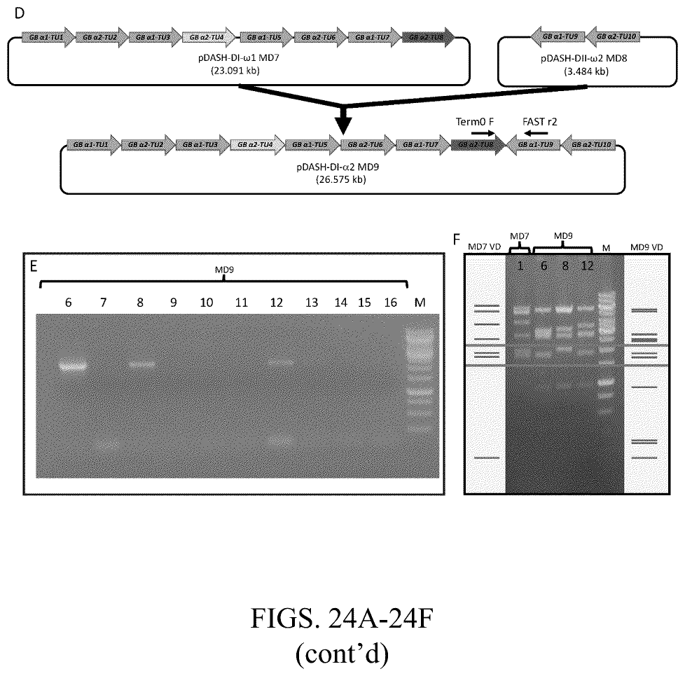

To test the gene stacking vectors, 8 transcriptional units (TUs) consisting of two CRISPRa systems (Streptococcus pyogenes (Spy) and S. thermophilus (Sth) based) in the donor vector Omega1-D1 (pDASH-DI-ω1) were assembled (FIG. 24A). The insert size was 23.167 kb. On one LB plate, there were 14 white colonies out of 51. Colony PCR using two pairs of primers showed that 9 out of 14 white colonies were positive (FIG. 24B). Mini-prep and digestion (using two different restriction enzymes, EcoRI and XhoI) showed that 7 plasmids are positive (out of 9 PCR positive colonies) (FIG. 24C). In other words, among 51 colonies, 7 are positive. Thus, the efficiency for assembling 8 TUs in a donor vector in this experiment is 13.7% among all colonies (7/51) or 50% of white colonies (7/14).

### Example 5: Assembly of 10 Transcriptional Units (TUs) in a Donor Vector

To further evaluate the assembly efficiency, the 8 TUs were assembled with two additional TUs (containing FAST and BASTA cassettes) (FIG. 24D). Twenty-five white colonies were picked for colony PCR. Three colonies were positive (FIG. 24E). The expectation was that this assembly will not work as the expected size of the insert (26.58 kb) exceeds the maximum cargo capacity of the donor vector.

Mini-prep of these 3 samples of 10 TUs and digestion (using NsiI), along with a control (8 TUs), indicated that a region was missing (FIG. 24F). The lack of correctly assembled constructs is consistent with the notion that the insert size of 10 TUs (26.58 kb) is over the limit of the vector's insert size, which caused the deletion or instability.

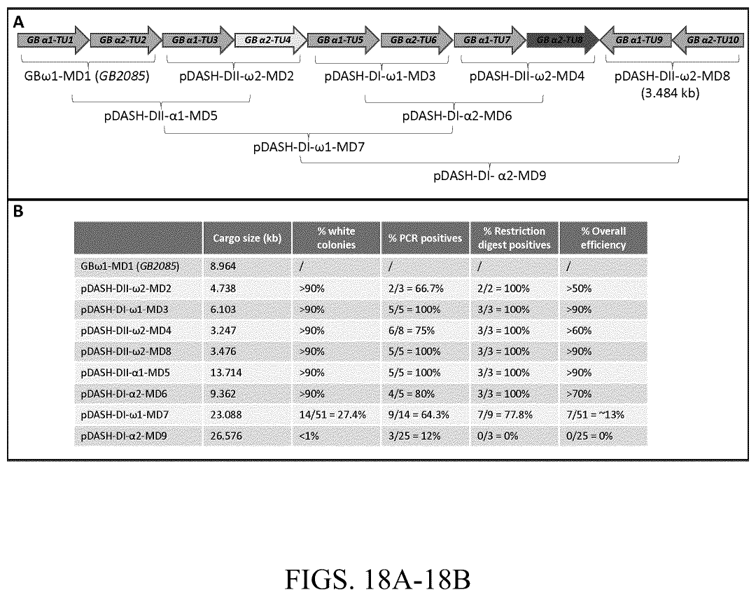

Overall, the assembly efficiency for 8 TUs was 13.7% for a 23.1 kb construct, while it was 0 for 10 TUs (a 26.58 kb construct) (FIGS. 18A-18B). This observation highlights the need to utilize BAC-based vectors that have a much greater cargo carrying capacity of up to 300 kb.

### Example 6: Integration of 8 Transcriptional Units (TUs) into the Acceptor Vector

Integration of the assembled 8 TUs (23.167 kb) into the acceptor vector was tested (FIG. 14). The new bacterial strain (CZ105a) containing the acceptor vector was electroporated with the 23.1 kb donor vector (with 8 TUs) and cells containing both vectors were selected in Kan plus Spec (FIG. 14). The integration of the donor into the acceptor was then induced by adding rhamnose to growth media, and the loss of the donor vector backbone was triggered upon addition of arabinose. Two sugar-mediated induction protocols were tested. a) Growing E. coli (with acceptor and donor vectors) in LB with Kan to OD600=0.6, adding rhamnose for 3 hours, then adding arabinose for another 3 hours, and streaking cells on LB+Kan; b) Growing E. coli in LB with two antibiotics (Kan for acceptor and Spec for donor vector) to OD600=0.6, adding rhamnose for 3 hours, removing LB, resuspending cells in LB with Kan, adding arabinose for another 3 hours, and streaking cells on LB+Kan. The second protocol worked better.

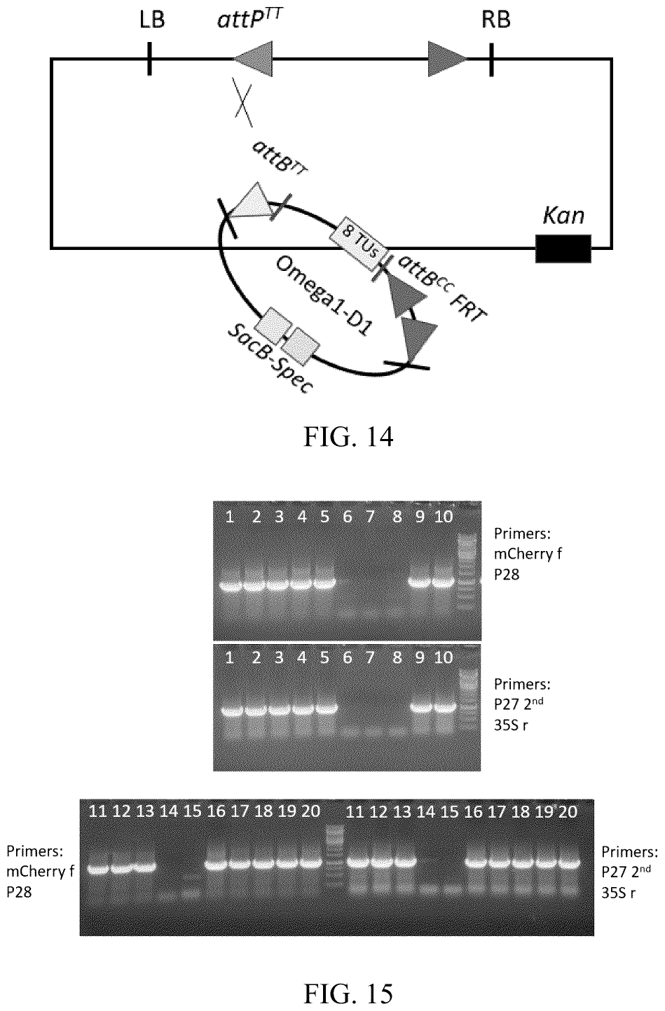

For recombination reactions performed using the latter protocol of recombinase induction, colony PCR was performed on colonies that grew on LB+Kan plates after the dual sugar treatment (FIG. 15). Two pairs of primers were used to test 20 randomly picked colonies, and 15 colonies (out of 20) were found to be positive by PCR. Thus, the integration efficiency of an 8 TU module from the donor to the acceptor was 75%.

### Example 7: Repeat Integration

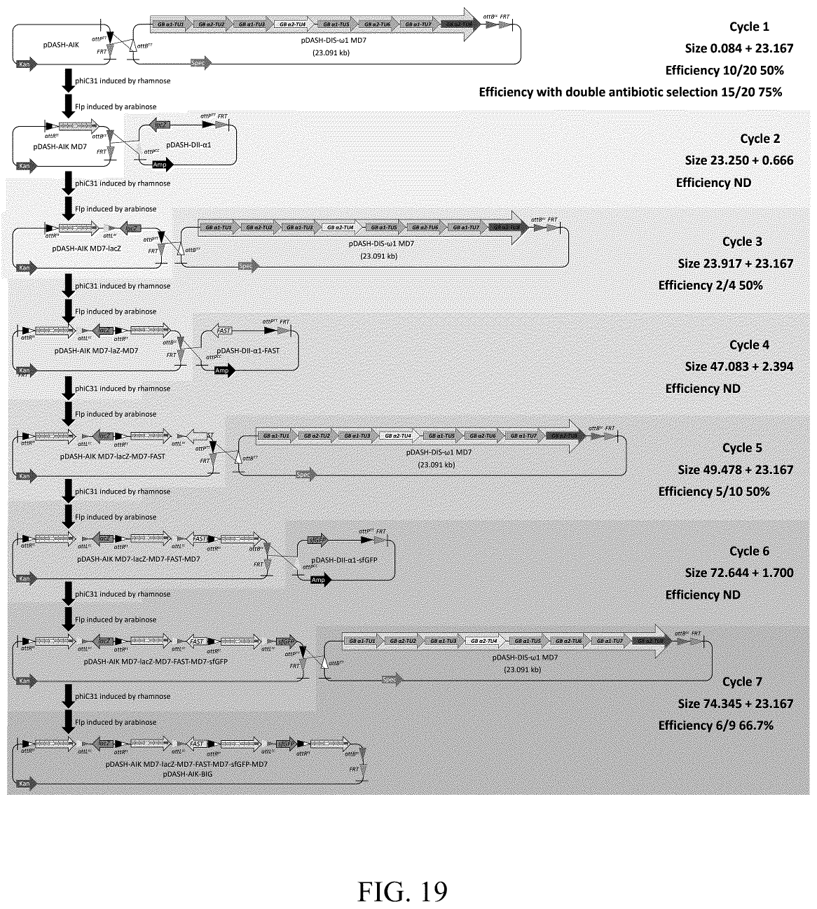

To test repeated integration, a construct with an insert of ˜100 kb consisting of four copies of the 23.1 kb 8 TU module linked together by three unique TUs (0.6 kb lacZ, 2.3 kb FAST, and 1.7 kb sfGFP) was built (FIG. 19).

### Example 8: Design of the DNA Assembly and Stacking Hybrid (DASH) System

The core DASH system consists of three basic components: the SW105-derived E. coli strain (CZ105a), a set of four donor vectors derived from the GoldenBraid assembly system (pDASH-DII-α1, pDASH-DI-α2, pDASH-DI-ω1, and pDASH-DII-ω2), and the pYLTAC17-derived acceptor vector (pDASH-AIK) (FIG. 17A). In addition to these basic components, several additional vectors were developed to expand the capabilities of the system. Thus, for example, the pDASH-DII-α1 (MoClo) and pDASH-DI-α2 (MoClo) were developed to illustrate how simple modification of the core donor plasmids could be used to increase the compatibility between DASH and other Type IIS-based DNA assembly systems. Similarly, to facilitate the removal of the donor plasmids and their derived backbone after the integration of the cargo into the acceptor vector, a second set of donor vectors containing the SacB counter-selectable marker was also created (FIG. 17B). In addition, a second acceptor vector, pDASH-AIIK, was generated to enable the initiation of the stacking process with a DII-type donor vectors instead of a DI.

The CZ105a DASH strain was derived from the recombincering strain SW105, which harbors a genomic insertion of a defective lambda prophage. This prophage includes the three lambda-Red recombineering-required genes: gam, beta, and exo. These genes are controlled by the temperature-sensitive c1857 repressor, which prevents or allows their expression at low (30° C.) and high (42° C.) temperatures, respectively. In addition, this strain also carries the arabinose-regulated araC transcription factor to drive the expression of the arabinose-inducible FLP recombinase gene, PBADflpe. The recombincering capabilities of the parental SW105 were utilized to insert the RBS-PhiC31 sequence downstream of the rhamnose-inducible genes rhaA and rhaT in the SW105 genome, creating the CZ105a and CZ105b DASH strains (FIGS. 21A-21B), respectively. This was achieved in a two-step recombinecring process. First, a positive-negative selection cassette RPSL-Amp was inserted just downstream of the rhaA stop codon and then replaced by the RBS-PhiC31 sequence to generate the CZ105a DASH strain in the second recombineering reaction. Using the same strategy, the RBS-PhiC31 sequence was also inserted immediately downstream of the stop codon of rhaT in the SW105 strain to generate the CZ105b DASH strain.

The PhiC31 catalyzes recombination between heterotypic sites, attP and attB. This reaction is irreversible as PhiC31 alone cannot catalyze the recombination between the resulting attR and attL sequences. If the attP and attB sites are in different plasmids, their recombination will combine both molecules to generate a single hybrid plasmid. The attP and attB sequences are both palindromic with a central nonpalindromic sequence. By changing this central nonpalindromic sequence, it is possible to generate orthogonal attP-attB site pairs. Thus, for example, the attP^{TT} and the attB^{TT}, where the ‘TT’ superscripts indicate the nonpalindromic central dinucleotide sequences, can recombine, whereas the attP^{TT} and attB^{CC} cannot. The orthogonality between ‘TT’ and ‘CC’ sites can be utilized for the iterative integration of donor vectors into an acceptor vector. Thus, for example, if a donor vector contains both attB^{TT} and attB^{CC} sites, it can be integrated into an acceptor vector containing an attP^{TT} site. The resulting hybrid plasmid will contain the attL^{TT} and attR^{TT} inactive sites as a result of the recombination between the attB^{TT} and attP^{TT} sites, as well as a functional intact attB^{CC} site. If the second donor vector has an attp^{CC} and attP^{TT} sites, it could be integrated into the hybrid vectors from the first integration cycle, producing inactive attL^{CC} and attR^{CC} and also introducing an active intact attP^{TT} site that would be available for the next round of integration, and so on. This strategy could, in principle, be used to insert as many donor vectors as necessary into the acceptor vector, but it would result in the accumulation of not only the cargo sequences present in the donor vectors but also the vector backbones. The removal of the backbone sequences of the donor vector from the hybrid plasmids can be achieved using another site-specific recombinase, FLP, which catalyzes the recombination between two homotypic FRT sites. If the two identical FRT sites are in the head-to-tail orientation in the same DNA molecule, the FLP recombinase precisely excises the sequence between the two sites, leaving a single copy of an active FRT. By strategically introducing FRT sites at selected locations in both the donor and acceptor vectors—one FRT in each—it becomes possible to remove the backbone of the donor vector after each attP-attB integration cycle.

Experiments were designed to take advantage of the properties of the PhiC31 and FLP recombinases and their corresponding recognition sites described above to modify the existing GoldenBraid donor and high-cargo capacity pYLTAC17 vectors to generate the set of DASH donor and acceptor plasmids. Thus, the attp^{CC}-attP^{TT}-FRT sequences were introduced into the GoldenBraid pDGB3α1 and pDGB3ω2 vectors to generate the pDASH-DII-α1 and the pDASH-DII-ω2 plasmids. Similarly, the attB^{TT}-attB^{CC}-FRT sequences were inserted into the GoldenBraid pDGB3ω1 and pDGB3α2 vectors to generate the pDASH-DI-ω1 and the pDASH-DI-α2 plasmids, respectively. To make the pDASH-DII-α1 and pDASH-DI-α2 compatible with the acceptor vectors (pDASH-AIK and pDASH-AIIK acceptors, see below), the Kan-resistance gene in pDASH alpha vectors was replaced with a BsaI-domesticated Amp-resistance gene (FIG. 17B, Tables 2-4). To facilitate the elimination of the pDASH donor vectors from the E. coli cells after each round of integration and excision, partial Shine-Dalgarno sequence (GGAGG) and spacer sequence (ACAATC) followed by the coding sequence of a BsaI-and BsmBI-domesticated SacB contra-selectable marker gene were inserted downstream of the stop codon of the Amp or Spec-resistance genes in each of the original pDASH donor vectors (FIG. 17B and Table 4). To make this system compatible with the widely used MoClo assembly system, the pDASH-DII-α1 and pDASH-DI-α2 donor vectors were modified such that the overhangs generated by the BsaI digestion become compatible with the MoClo level M transcriptional units (FIG. 17B). These MoClo-compatible vectors were referred to as pDASH-DII-α1 (MoClo) and pDASH-DI-α2 (MoClo) (Table 4). Finally, to generate the pDASH-AIK and pDASH-AIIK acceptor vectors, the attP^{TT}-FRT and attB^{CC}-FRT sequences, respectively, were introduced into the large cargo capacity vector pYLTAC17 (FIG. 17, Table 2).

### Example 9: Functionality of the Rhamnose-Inducible PhiC31 Expressed from a Genomic Locus

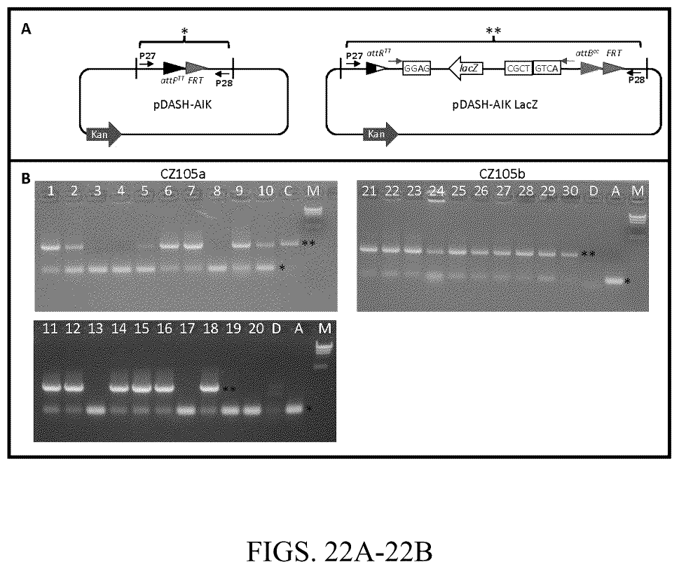

In E. coli, the rha regulon, which contains a rhamnose transporter gene rhaT, the rhaBAD operon for rhamnose catabolismand the rhaRS regulatory operon, is responsible for L-rhamnose metabolism. Two promoters in this regulon, PrhaBAD and PrhaT, have been shown to be tightly controlled by rhamnose and extensively used in plasmid-based inducible systems in bacteria. To determine if PhiC31 driven by these rhamnose-inducible promoters in their chromosomal context was effective at catalyzing the recombination between attP and attB sites in E. coli, two DASH E. coli strains, CZ105a and CZ105b, were transformed (harboring the rhamnose-inducible PhiC31 gene) (FIGS. 21A-21B) with the pDASH-DI-ωl donor and pDASH-AIK acceptor vectors. After growing in the presence of both spectinomycin and kanamycin in liquid LB to an OD₆₀₀ of ˜0.6, the cells were pelleted and resuspended in LB containing kanamycin and 0.1% W/V L-rhamnose to induce the expression of the PhiC31 recombinase gene. After 3 h at 30° C. and constant shaking, L-arabinose was added to the culture to a final concentration of 0.1%, and the cultures were incubated for another 3 h at 30° C. and constant shaking to induce the FLP expression. It was observed that the CZ105a cells grew normally, while CA105b did so slowly. After plating the induced cells in LB-kanamycin plates and incubating the cells for two days at 30° C., colony PCR was performed using the diagnostic primers P27 and P28 (FIGS. 22A-22B, and Table 2). PCR and subsequent Sanger sequencing showed that in ˜50% of colonies in the CZ105a background (10 out of 20 colonies), the LacZ fragment from the donor vector was correctly integrated into the acceptor vector, and the corresponding backbone was removed (FIG. 23A). The fidelity of the two recombination events was confirmed by Sanger sequencing of the integration junctions (FIG. 23B). On the other hand, 100% of colonies of CZ105b still contained the intermediate plasmid, in which the donor vector was integrated into the acceptor vector, but the excision of the donor vector backbone could not be confirmed. Given the poor growth of CZ105b and the unexpected difficulties with excising the backbone of the donor vector, it was decided to do all the subsequent experiments with the CZ105a strain.

To demonstrate the system's capability of supporting several rounds of integration and excision, the CZ105a cells carrying the pDASH-AIK-lacZ from the first donor integration cycle were transformed with the pDASH-DII-02 donor vector. After following the same L-rhamnose and L-arabinose induction protocol described above, cells were plated in kanamycin-containing LB plates, and the integration and excision recombination products were examined by colony PCR. As with the first integration cycle, PCR screening with the diagnostic primers P27 and P28 (FIG. 23 and Table 2) indicated that ˜50% of the colonies had undergone a correct second round of integration-excision recombination. As before, the fidelity of this recombination was confirmed by Sanger sequencing (FIG. 23B).

### Example 10: Compatibility of the DASH and GoldenBraid Systems

As mentioned earlier, the DASH system can be used to assemble parts and stack transcriptional units from various Golden Gate-based systems, including GoldenBraid, Loop, Mobius, MoClo, and Start-Stop. This compatibility can easily be expanded by using linkers or modified pDASH donor vectors, such as pDASH-DII-α1 (MoClo) and pDASH-DI-α2 (MoClo) (FIG. 17B). To demonstrate the compatibility of the DASH system and its utility to bypass the cargo size limitation of other platforms, eight transcriptional units were combined in a series of binary assembly reactions using a combination of GoldenBraid and DASH vectors. These eight transcriptional units had been previously assembled in the GoldenBraid GBα1 (35S-MCP-VPR-Tnos [TU1]), AtU6p-sgRNA_{Spy(SIDFR)}-MS2-A1U6t [TU3], 35S-dCas9_{Sth}-EDLL-T35 #0 [TU5], AtU6p-sgRNA_{Sth(ADH1)}-MS2-A1U6t [TU7]) or in GBα2 (35S-dCas9_{Spy}-EDLL-Tnos [TU2]), SIDFRP_{Spy(SIDFR)}-3xYpet-Term35S #0 [TU4], Kan (GB0184) [TU6], SIDFRP_{Sth(ADH1)}-mCherry-T35S #0 [TU8]) (FIG. 18). Using standard (Golden Gate) assembly protocols, the TU1 and TU2 were combined into the GoldenBraid pDGB3 ωl (GBω1) vector to generate the GBω1-MD1, the TU3 and TU4 were combined into the pDASH-DII-ω2 vector to form the pDASH-DII-ω2-MD2. Similarly, the TU5 plus TU6, and TU7 plus TU8 were combined to form the pDASH-DI-ω1-MD3 and pDASH-DII-ω2-MD4, respectively. Again, using the typical braiding assembly strategy of GoldenBraid, the GBω1-MD1 and the pDASH-DII-ω2-MD2 were assembled into the pDASH-DII-α1 vector to form the pDASH-DII-α1-MD5 module, while the pDASH-DI-ω1-MD3 and pDASH-DII-ω2-MD4 were combined into the pDASH-DI-α2 to form the pDASH-DI-α2-MD6 module. It is important to point out that all these assemblies worked with the typical high efficiency of the GoldenBraid cloning system, even though a mixture of vectors was used from both platforms, the original GoldenBraid pDGB3 and the DASH vectors. Next, the pDASH-DII-α1-MD5 and pDASH-DI-α2-MD6 were combined into the pDASH-DI-ω1 to form the pDASH-DI-ω1-MD7 (FIG. 18). As typically observed when generating constructs of over 15-20 kb in GoldenBraid pDGB3 vectors, a significant decrease in efficiency was noted in the assembly of the ˜23 kb pDASH-DI-ω1-MD7, where only ˜13% of the colonies had the expected module combination based on LacZ activity, colony PCR using the primers mCherry f and pDGB3 r, and restriction digestion diagnosis (Tables 5-6, and FIGS. 24A-24C). The size of the pDASH-DI-ω1-MD7 was further increased by combining it with the 3.5 kb pDASH-DII-ω2-MD8 containing the FAST and BASTA (GB0023) transcriptional units to create pDASH-DI-α2-MD9. However, after screening 25 white colonies by colony PCR and the following restriction digestion, positives colonies were not identified (Tables 5-6, FIGS. 24D-24F).

### Example 11: High Cargo Capacity of the DASH Platform

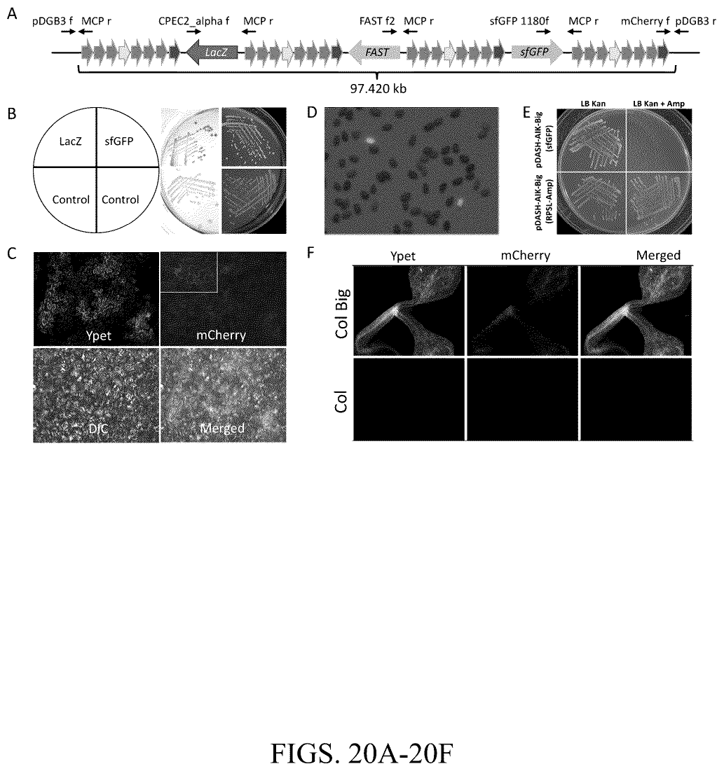

The difficulties with assembling a ˜26 kb construct and the low efficiency observed in the construction of the ˜23 kb pDASH-DI-ω1-MD7 illustrate the limitations of the standard GoldenBraid assembly system in generating large, multigenic constructs. To mitigate this limitation, the DASH system was equipped with two special features. On the one hand, the PhiC31 recombinase present in the DASH CZ105a strain has, in principle, the capacity to combine large DNA molecules as it does during the normal life cycle of phages, where it catalyzes the integration of the ˜42 kb phage DNA into the large size bacterial host genome. On the other hand, the pYLTAC17-derived pDASH-AIK acceptor vector has a cargo capacity of over 100 kb, allowing for the stable propagation of large multigenic constructs. To test the capability of the DASH system to operate with large DNA fragments, experiments were conducted to examine the efficiency of transferring the 23 kb cargo from the pDASH-DI-01-MD7 to the pDASH-AIK acceptor vector. Competent cells of the CZ105a strain carrying the empty pDASH-AIK were transformed with the purified pDASH-DI-ω1-MD7 plasmid. Cells were resuspended in LB containing kanamycin, and PhiC31 and FLP were induced as described above. Eleven out of 20 (55%) of the randomly selected colonies showed the correct integration of the 23 kb cargo and excision of the corresponding backbone sequences of the pDASH-DI-ω1-MD7 module based on PCR using the diagnostic primers P27 2^{nd} and 35S r, mCherry f and P28 (Tables 2, 5, 6, and FIG. 19). This efficiency was similar to that previously observed when integrating the much smaller lacZ cargo, indicating that the size of the DNA fragments transferred from the DASH donor to the DASH acceptor vectors did not affect the efficiency of the integration/excision process. To further confirm that was the case and to test the potential influence of the cargo size in the DASH acceptor vector on the integration efficiency, six additional rounds of integration/excision were carried out (FIG. 19), alternating incorporation of a unique-sequence single transcriptional unit and a module of eight transcriptional units of the pDASH-DI-ω1-MD7. Thus, in the subsequent rounds of integration excision, the donor vectors pDASH-DII-α1 (containing LacZ), pDASH-DI-ω1-MD7, pDASH-DII-α1-FAST, pDASH-DI-ω1-MD7, pDASH-DIIS-α1-sfGFP, and pDASH-DI-ω1-MD7 were used to generate the 35-transcriptional unit pDASH-AIK-Big construct (FIG. 19). Importantly, the integration/excision efficiency remained constant at around 50% (FIG. 19), confirming that neither the size of the donor nor acceptor vectors have a significant effect on the efficiency of the stacking system. The fidelity of the integration excision process was confirmed by diagnostic PCR using five pairs of primers (FIG. 20A) and whole-plasmid sequencing (plasmidsaurus.com).

Although the ˜50% efficiency observed in these experiments is more than sufficient to demonstrate the practical utility of the system, future systematic optimization of the protocol is likely to result in even greater overall efficiencies. In fact, during the first cycle of integration/excision, when the cargo of the pDASH-DI-ω1-MD7 was transferred to the pDASH-AIK, a second antibiotic was added to the media for the selection of the donor vector during rhamnose induction. This adjustment resulted in an increase in efficiency from 50% to 75%. These encouraging results suggest that additional simple protocol optimizations, such as induction times, culture conditions, etc., could further improve the already high efficiency of the system.

### Example 12: Stability and in Planta Functionality of the DASH-Generated Constructs

To determine the functionality of the constructs generated using the DASH platform, experiments were designed and conducted to examine the activity of the genes present in the 97 kb pDASH-AIK-Big construct described above (FIG. 19). Of the 35 transcriptional units of this construct (FIG. 20A), LacZ and sfGFP should be functional in E. coli, while the rest should be active in plants. The functionality of LacZ and sfGFP was assessed by growing CZ105a cells carrying the pDASH-AIK-Big construct on LB plates supplemented with X-gal and by imaging the fluorescence of bacterial colonies under blue light, respectively. As can be observed in FIG. 20B, both genes were active in E. coli. The rest of the 33 genes present in the pDASH-AIK-Big correspond to four identical copies of an eight transcriptional unit module and the FAST marker gene. In this module, two different dCas9-EDLL activation domain fusion proteins (one from Streptococcus pyogenic (Spy) and another from S. thermophilus (Sth)), the corresponding gRNA-MS2 fusions, and the MCP-VPR fusion protein are used to drive the expression of two reporter genes, 3xYpet and mCherry (FIG. 18). The expression of the 3xYPet requires the activities of the transcriptional units TU1, 2, and 3, while the expression of the mCherry requires the activity of TU1, 5, and 7. In addition, the TU6 should confer kanamycin resistance in the corresponding transgenic plants (FIG. 18), while the FAST transcriptional unit (FIG. 19) should result in red, fluorescent seed coats. Thus, to examine the functionality of these genes in plants, the activity of the 3xYpet and mCherry was first evaluated in N. benthamiana transient expression assays. For that, the pDASH-AIK-Big was purified from CZ105a E. coli and electroporated into Agrobacterium GV3101. After selecting transformants in LB kanamycin plates and confirming the presence of the pDASH-AIK-Big construct by PCR using five pairs of diagnostic primers (Table 6), positive clones were grown in liquid media and used for agroinfiltration of N. benthamiana leaves. Three days after infiltration, the activity of the two fluorescent proteins was examined using fluorescence microscopy. The expression of both 3xYPet and mCherry (FIG. 20C) confirms the functionality of these two reporter genes and, therefore, that of the five other transcriptional units required for their activation. In addition, these experiments show the feasibility of using the pDASH-AIK acceptor vector in transient expression in N. benthamiana, even when the DNA cargos are very large, like in the current example.

In addition to testing the functionality of the pDASH-AIK-Big in transient assays, Arabidopsis stable transgenic lines were also generated using the standard floral dip method. First, the transformation efficiency was estimated by determining the percentage of red, fluorescent seeds among the population of seeds from TO plants (FIG. 20D). The average efficiency of transformation in two independent experiments was much lower than when using standard binary vectors and just around 0.1%. These results agree with previous observations using the original PYLTAC17 vector. The rest of the nonfluorescent seeds of the TO plants were also screened for kanamycin resistance. Additional transformants were not found, indicating that the FAST selection was effective, and the transformation efficiency estimated using this marker was accurate. Both Ypet and mCherry fluorescence were also detected in the transgenic plants obtained (FIG. 20D and 20F). As with the transient expression in N. benthamiana, the detection of fluorescence indicates the functionality of the ˜97 kb T-DNA in stable transformants.

### Example 13: Post-Assembly Modification Capability of the DASH System

One of the key characteristics of the synthetic biology approach is its distinctive “design, build, test, learn” cycle. This engineering process often requires the redesign of specific DNA parts after evaluating their performance, often in the context of multigenic constructs. The development of a highly efficient assembly system greatly facilitates the process of generating these modifications by simply replacing one of the parts in a simple single-gene or small multigene assembly reaction. Redesigning constructs becomes considerably more challenging when dealing with large multigenic assemblies, as replacing a single component often requires restarting the entire assembly process, which typically involves multiple assembly cycles. Recombineering presents an attractive solution to this problem, as it allows for the introduction of any modifications (insertions, deletions, or replacements) in sequences of practically any size with high efficiency and precision. Thus, to facilitate the introduction of post-assembly modifications in the potentially large DASH constructs, the system has built-in recombineering capabilities. To demonstrate the functionality of this feature in the DASH, the coding sequence of the sfGFP in the 97 kb cargo of pDASH-AIK-Big was replaced with the RPSL-Amp cassette sequences. The 40 bp sequences flanking the sfGFP were incorporated at the ends of the RPSL-Amp recombineering cassette by PCR using the primers RPSL-Amp rep sfGFP f and RPSL-Amp rep sfGFP r. This PCR product was electroporated into recombineering-competent CZ105a cells carrying the 116 kb pDASH-AIK-Big construct. Ampicillin and kanamycin-resistant cells were selected in LB plates, and the replacement of sfGFP with RPSL-Amp was confirmed by PCR as well as functional assays (FIG. 20E, Table 6)

### Example 14: Sequences

Table 7 provides sequences of the present disclosure.

TABLE 7
Sequences

| | | SEQ |
|---|---|---|
| Description | Sequence | ID NO: |
| PhiC31 (605 | atggacacgtacgcgggtgcttacgaccgtcagtcgcgcgagcgcgaaaattcgagcgcagca | 1 |
| aa version) | agcccagcgacacagcgtagcgccaacgaagacaaggcggccgaccttcagcgcgaagtcga | |
| | gcgcgacgggggccggttcaggttcgtcgggcatttcagcgaagcgccgggcacgtcggogtt | |
| | cgggacggcggagcgcccggagttcgaacgcatcctgaacgaatgccgcgccgggcggctca | |
| | acatgatcattgtctatgacgtgtcgcgcttctcgcgcctgaaggtcatggacgcgattccgattgtc | |
| | tcggaattgctcgccctgggcgtgacgattgtttccactcaggaaggcgtcttccggcagggaaa | |
| | cgtcatggacctgattcacctgattatgcggctcgacgcgtcgcacaaagaatcttcgctgaagtc | |
| | ggcgaagattctcgacacgaagaaccttcagcgcgaattgggcgggtacgtcggcgggaaggc | |
| | gccttacggcttcgagcttgtttcggagacgaaggagatcacgcgcaacggccgaatggtcaatg | |
| | tcgtcatcaacaagcttgcgcactcgaccactccccttaccggacccttcgagttcgagcccgacg | |
| | taatccggtggtggtggcgtgagatcaagacgcacaaacaccttcccttcaagccgggcagtcaa | |
| | gccgccattcacccgggcagcatcacggggctttgtaagcgcatggacgctgacgccgtgccga | |
| | cccggggcgagacgattgggaagaagaccgcttcaagcgcctgggacccggcaaccgttatgc | |
| | gaatccttcgggacccgcgtattgcgggcttcgccgctgaggtgatctacaagaagaagccgga | |
| | cggcacgccgaccacgaagattgagggttaccgcattcagcgcgacccgatcacgctccggcc | |
| | ggtcgagcttgattgcggaccgatcatcgagcccgctgagtggtatgagcttcaggcgtggttgg | |
| | acggcagggggcgcggcaaggggctttcccgggggcaagccattctgtccgccatggacaag | |
| | ctgtactgcgagtgtggcgccgtcatgacttcgaagcgcggggaagaatcgatcaaggactctta | |
| | ccgctgccgtcgccggaaggtggtcgacccgtccgcacctgggcagcacgaaggcacgtgca | |
| | acgtcagcatggcggcactcgacaagttcgttgcggaacgcatcttcaacaagatcaggcacgc | |
| | cgaaggcgacgaagagacgttggcgcttctgtgggaagccgcccgacgcttcggcaagctcac | |
| | tgaggcgcctgagaagagcggcgaacgggcgaaccttgttgcggagcgcgccgacgccctga | |
| | acgcccttgaagagctgtacgaagaccgcgcggcaggcgcgtacgacggacccgttggcagg | |
| | aagcacttccggaagcaacaggcagcgctgacgctccggcagcaaggggcggaagagcggc | |
| | ttgccgaacttgaagccgccgaagccccgaagcttccccttgaccaatggttccccgaagacgcc | |
| | gacgctgacccgaccggccctaagtcgtggtgggggcgcgcgtcagtagacgacaagcgcgt | |
| | gttcgtcgggctcttcgtagacaagatcgttgtcacgaagtcgactacgggcagggggcaggga | |
| | acgcccatcgagaagcgcgcttcgatcacgtgggcgaagccgccgaccgacgacgacgaaga | |
| | cgacgcccaggacggcacggaagacgtagcggcgtag | |
| Domesticated | ATGAGTATTCAACATTTCCGTGTCGCCCTTATTCCCTTTT | 2 |
| Amp-SacB | TTGCGGCATTTTGCCTTCCTGTTTTTGCTCACCCAGAAA | |
| (in one | CGCTGGTGAAAGTAAAAGATGCTGAAGATCAGTTGGGT | |
| operon) | GCACGAGTGGGTTACATCGAACTGGATCTCAACAGCGG | |
| sequence. | TAAGATCCTTGAGAGTTTTCGCCCCGAAGAACGTTTTCC | |
| The | AATGATGAGCACTTTTAAAGTTCTGCTATGTGGCGCGGT | |
| domesticated | ATTATCCCGTATTGACGCCGGGCAAGAGCAACTCGGTCG | |
| ampicillin | CCGCATACACTATTCTCAGAATGACTTGGTTGAGTACTC | |
| (Amp) | ACCAGTCACAGAAAAGCATCTTACGGATGGCATGACAG | |
| resistance | TAAGAGAATTATGCAGTGCTGCCATAACCATGAGTGATA | |
| (marked in | ACACTGCGGCCAACTTACTTCTGACAACGATCGGAGGA | |
| bold) and | CCGAAGGAGCTAACCGCTTTTTTGCACAACATGGGGGA | |
| SacB | TCATGTAACTCGCCTTGATCGTTGGGAACCGGAGCTGA | |
| (marked in | ATGAAGCCATACCAAACGACGAGCGTGACACCACGATG | |
| italics) | CCTGTAGCAATGGCAACAACGTTGCGCAAACTATTAACT | |
| genes were | GGCGAACTACTTACTCTAGCTTCCCGGCAACAATTAATA | |
| linked into | GACTGGATGGAGGCGGATAAAGTTGCAGGACCACTTCT | |
| one operon | GCGCTCGGCCCTTCCGGCTGGCTGGTTTATTGCTGATAA | |
| by the | ATCTGGAGCCGGTGAGCGTGGGTCcCGCGGTATCATTGC | |
| optimal SD | AGCACTGGGGCCAGATGGTAAGCCCTCCCGTATCGTAG | |
| sequence | TTATCTACACGACGGGGAGTCAGGCAACTATGGATGAAC | |
| and a spacer | GAAATAGACAGATCGCTGAGATAGGTGCCTCACTGATTA | |
| sequence, | AGCATTGGtaaGGAGGACAATCATGAACATGAACATCAAAAAA | |
| which was | ATTGTAAAACAAGCCACAGTTCTGACTTTTACGACTGCACTTCTG | |
| used to | GCAGGAGGAGCGACTCAAGCCTTCGCGAAAGAAAATAACCAAA | |
| replace the | AAGCATACAAAGAAACGTACGGCGTITCTCATATTACACGCCATG | |
| original | ATATGCTGCAGATCCCTAAACAGCAGCAAAACGAAAAATACCAA | |
| kanamycin | GTGCCTCAATTCGATCAATCAACGATTAAAAATATTGAGTCTGCA | |
| (Kan) | AAAGGACTTGATGTGTGGGACAGCTGGCCGCTGCAAAACGCTG | |
| resistance | ACGGAACAGTAGCTGAATACAACGGCTATCACGTTGTGTTTGCT | |
| gene. The | CTTGCGGGAAGCCCGAAAGACGCTGATGACACATCAATCTACA | |
| start and | TGTTTTATCAAAAGGTCGGCGACAACTCAATCGACAGCTGGAAA | |
| stop codons | AACGCGGGCCGTGTCTTTAAAGACAGCGATAAGTTCGACGCCA | |
| of Amp and | ACGATCCGATCCTGAAAGATCAGACGCAAGAATGGTCCGGTTC | |
| SacB genes | TGCAACCTTTACATCTGACGGAAAAATCCGTTTATTCTACACTGA | |
| are | CTATTCCGGTAAACATTACGGCAAACAAAGCCTGACAACAGCGC | |
| underlined. | AGGTAAATGTGTCAAAATCTGATGACACACTCAAAATCAACGGA | |
| | GTGGAAGATCACAAAACGATTTTTGACGGAGAIGGAAAAACATAT | |
| | CAGAACGTTCAGCAGTTTATCGATGAAGGCAATTATACATCCGG | |
| | CGACAACCATACGCTGAGAGAICCTCACTACGTTGAAGACAAAG | |
| | GCCATAAATACCTTGTATTCGAAGCCAACACGGGAACAGAAAAC | |
| | GGATACCAAGGCGAAGAATCTTTATTTAACAAAGCGTACTACGG | |
| | CGGCGGCACGAACTTCTTCCGTAAAGAAAGCCAGAAGCTTCAG | |
| | CAGAGCGCTAAAAAACGCGATGCTGAGTTAGCGAACGGCGCCC | |
| | TCGGTATCATAGAGTTAAATAATGATTACACATTGAAAAAAGTAAT | |
| | GAAGCCGCTGATCACTTCAAACACGGTAACTGATGAAATCGAG | |
| | CGCGCGAATGTTTTCAAAATGAACGGCAAATGGTACTTGTTCAC | |
| | TGATTCACGCGGTTCAAAAATGACGATCGATGGTATTAACTCAAA | |
| | CGATATTTACATGCTTGGTTATGTATCACACTCTTTAACCGGCCC | |
| | TTACAAGCCGCTGAACAAAACAGGGCTTGTGCTGCAAATGGGT | |
| | CTTGATCCAAACGATGTGACATTCACTTACTCTCACTTCGCAGT | |
| | GCCGCAAGCCAAAGGCAACAATGTGGTTATCACAAGCTACATGA | |
| | CAAACAGAGGCTTCTTCGAGGATAAAAAGGCAACATTTGCGCCA | |
| | AGCTTCTTAATGAACATCAAAGGCAATAAAACATCCGTTGTCAAA | |
| | AACAGCATCCTGGAGCAAGGACAGCTGACAGTCAACTAA | |
| Domesticated | taaGGAGGACAATCATGAACATGAACATCAAAAAAATTGTAAAAC | 3 |
| SacB | AAGCCACAGTTCTGACTTTTACGACTGCACTTCTGGCAGGAGG | |
| sequence. | AGCGACTCAAGCCTTCGCGAAAGAAAATAACCAAAAAGCATACA | |
| Partial | AAGAAACGTACGGCGTITCTCATATTACACGCCATGATATGCTGC | |
| Shine- | AGATCCCTAAACAGCAGCAAAACGAAAAATACCAAGTGCCTCAA | |
| Dalgarno | TTCGATCAATCAACGATTAAAAATATTGAGTCTGCAAAAGGACTT | |
| sequence | GATGTGTGGGACAGCTGGCCGCTGCAAAACGCTGACGGAACA | |
| (GGAGG) | GTAGCTGAATACAACGGCTATCACGTTGTGTTTGCTCTTGCGGG | |
| and spacer | AAGCCCGAAAGACGCTGATGACACATCAATCTACATGTTTTATCA | |
| sequence | AAAGGTCGGCGACAACTCAATCGACAGCTGGAAAAACGCGGG | |
| (ACAATC) | CCGTGTCTTTAAAGACAGCGATAAGTTCGACGCCAACGATCCGA | |
| followed by | TCCTGAAAGATCAGACGCAAGAATGGTCCGGTTCTGCAACCTTT | |
| SacB | ACATCTGACGGAAAAATCCGTTTATTCTACACTGACTATTCCGGT | |
| (marked in | AAACATTACGGCAAACAAAGCCTGACAACAGCGCAGGTAAATGT | |
| italics) were | GTCAAAATCTGATGACACACTCAAAATCAACGGAGTGGAAGATC | |
| inserted | ACAAAACGATTTTTGACGGAGAtGGAAAAACATATCAGAACGTTC | |
| downstream | AGCAGTTTATCGATGAAGGCAATTATACATCCGGCGACAACCAT | |
| of the stop | ACGCTGAGAGAICCTCACTACGTTGAAGACAAAGGCCATAAATA | |
| codon of the | CCTTGTATTCGAAGCCAACACGGGAACAGAAAACGGATACCAA | |
| spectinomycin | GGCGAAGAATCTTTATTTAACAAAGCGTACTACGGCGGCGGCAC | |
| (Spec) | GAACTTCTTCCGTAAAGAAAGCCAGAAGCTTCAGCAGAGCGCT | |
| resistance | AAAAAACGCGATGCTGAGTTAGCGAACGGCGCCCTCGGTATCA | |
| gene. The | TAGAGTTAAATAATGATTACACATTGAAAAAAGTAATGAAGCCGC | |
| start and | TGATCACTTCAAACACGGTAACTGATGAAATCGAGCGCGCGAAT | |
| stop codons | GTTTTCAAAATGAACGGCAAATGGTACTTGTTCACTGATTCACG | |
| of SacB are | CGGTTCAAAAATGACGATCGATGGTATTAACTCAAACGATATTTA | |
| underlined. | CATGCTTGGTTATGTATCACACTCTTTAACCGGCCCTTACAAGCC | |
| | GCTGAACAAAACAGGGCTTGTGCTGCAAATGGGTCTTGATCCA | |
| | AACGATGTGACATTCACTTACTCTCACTTCGCAGTGCCGCAAGC | |
| | CAAAGGCAACAATGTGGTTATCACAAGCTACATGACAAACAGAG | |
| | GCTTCTTCGAGGATAAAAAGGCAACATTTGCGCCAAGCTTCTTA | |
| | ATGAACATCAAAGGCAATAAAACATCCGTTGTCAAAAACAGCAT | |
| | CCTGGAGCAAGGACAGCTGACAGTCAACTAA | |

The invention claimed is:

1. A gene stacking system comprising:
a) at least one donor vector for delivering target DNA inserts;
b) a first acceptor vector configured to contain up to about 300 kb of target DNA inserts; and
c) a first engineered bacterial cell comprising the first acceptor vector.

2. The gene stacking system of claim 1, wherein the system comprises:
i) two donor vectors;
ii) four donor vectors; or
iii) eight donor vectors.

3. The gene stacking system of claim 1 or 2, further comprising a second acceptor vector configured to contain up to about 300 kb of target DNA inserts and a second engineered bacterial cell comprising the second acceptor vector.

4. (canceled)

5. The gene stacking system of claim 1, wherein the first engineered bacterial cell comprises a recombineering system.

6-8. (canceled)

9. The gene stacking system of claim 1, wherein the first engineered bacterial cell comprises an arabinose-inducible FLP recombinase.

10. (canceled)

11. The gene stacking system of claim 1, wherein the first engineered bacterial cell comprises an inducible PhiC31 recombinase or a rhamnose-inducible PhiC31 recombinase.

12. (canceled)

13. The gene stacking system of claim 1, wherein the first engineered bacterial cell is derived from an E. coli SW105 strain.

14-20. (canceled)

21. The gene stacking system of claim 1, wherein each of the donor vectors comprises a first att site, a second att site, and an FRT site; and/or wherein each of the donor vectors comprises an SacB gene.

22-25. (canceled)

26. The gene stacking system of claim 1, wherein the system is compatible with a GV3101 strain of Agrobacterium or a GV3101 derivate with a deletion in the mltB3 gene.

27. The gene stacking system of claim 1, wherein each of the donor vectors is either a Donor I vector or a Donor II vector and wherein the system is configured so that delivery of the target DNA inserts comprises introducing a Donor I vector or a Donor II vector into the first engineered bacterial cell or the second engineered bacterial cell.

28. (canceled)

29. The gene stacking system of claim 1, wherein the gene stacking system is compatible with Type IIS restriction enzyme-based cloning technologies in plants.

30. The gene stacking system of claim 1, wherein each of the donor vectors is configured to incorporate target DNA inserts from:
(i) Golden Gate vectors, GoldenBraid plasmids, Mobius Level-2 plasmids, and Loop pEven plasmids;
(ii) MoClo plasmids; or
(iii) either (i) or (ii).

31. A gene stacking system comprising:
a) a first donor vector for delivering target DNA inserts;
b) a second donor vector for delivering target DNA inserts;
c) an acceptor vector configured to contain up to about 300 kb of target DNA inserts; and
d) an engineered bacterial cell comprising the acceptor vector, wherein the engineered bacterial cell is configured to express at least one recombinase.

32. The gene stacking system of claim 31, wherein the first donor vector is configured to deliver at least a first target DNA insert to the acceptor vector and the second donor vector is configured to deliver at least a second target DNA insert to the acceptor vector.

33. The gene stacking system of claim 31 or 32, wherein the first donor vector is configured to deliver multiple of the same or different target DNA inserts to the acceptor vector via one or more rounds of integration and the second donor vector is configured to deliver multiple of the same or different target DNA inserts to the acceptor vector via one or more rounds of integration.

34-38. (canceled)

39. The gene stacking system of claim 31, wherein:
i) the first donor vector comprises attB recombination donor sites, the second donor vector comprises attP recombination donor sites, and the acceptor vector comprises an attP recombination acceptor site; or
ii) the first donor vector comprises attP recombination donor sites, the second donor vector comprises attB recombination donor sites, and the acceptor vector comprises an attB recombination acceptor site.

40-56. (canceled)

57. The gene stacking system of claim 31, wherein the first donor vector and/or the second donor vector are configured to incorporate target DNA inserts from: (i) Golden Gate vectors, GoldenBraid plasmids, Mobius Level-2 plasmids, and Loop pEven plasmids; or (ii) MoClo plasmids.

58. A gene stacking system comprising:
at least one donor vector comprising at least one target DNA insert flanked by recombination donor sites;
at least one acceptor vector comprising recombination acceptor sites compatible with the recombination donor sites of the at least one donor vector, wherein the at least one acceptor vector is configured to contain up to about 300 kb of target DNA insert; and
an engineered bacterial cell comprising the at least one acceptor vector, wherein the engineered bacteria cell is configured to express at least one recombinase.

59. The gene stacking system of claim 58, wherein expression of the at least one recombinase by the engineered bacterial cell is inducible.

60. The gene stacking system of claim 58 or 59, wherein the engineered bacterial cell further comprises a recombineering system.

59-87. (canceled)
